(19) United States
(12) Patent Application Publication
(10) Pub. No.: US 2017/0362324 A1
(43) Pub. Date: Dec. 21, 2017

(54) HUMANIZED ANTI-ALPHA V BETA 5 ANTIBODIES AND USES THEREOF

(71) Applicant: Biogen MA Inc., Cambridge, MA (US)

(72) Inventors: Thomas Owen Cameron, Cambridge, MA (US); Michael Adam Crackower, Needham, MA (US); Brian M. Dolinski, Franklin, MA (US); Karl J.M. Hanf, Billerica, MA (US); Amy Theresa McCurley, Boston, MA (US); Nels Eric Pederson, Mansfield, MA (US); Martin Preyer, Somerville, MA (US); Fang Qian, Southborough, MA (US); Shelia M. Violette, Lexington, MA (US); Paul Henry Weinreb, Andover, MA (US)

(21) Appl. No.: 15/510,422

(22) PCT Filed: Sep. 11, 2015
(86) PCT No.: PCT/US15/49746

§ 371 Date: Mar. 10, 2017

Related U.S. Application Data
(60) Provisional application No. 62/049,987, filed on Sep. 12, 2014.

(51) Int. Cl. C07K 16/28

(52) U.S. Cl. CPC C07K 16/2839; C07K 2317/92; C07K 2317/76; C07K 2317/24; C07K 2317/55; C07K 2317/41; C07K 2317/565

(57) ABSTRACT

Humanized antibodies and antibody fragments that bind to αvβ5 are disclosed. Also disclosed are methods of using the disclosed antibodies and antibody fragments to treat or prevent αvβ5-mediated diseases.

### CROSS-REFERENCE TO RELATED APPLICATIONS

This application claims the benefit of priority of U.S. Provisional Patent Application No. 62/049,987, filed Sep. 12, 2014, the contents of which are incorporated by reference in its entirety herein.

### FIELD

This invention relates generally to humanized antibodies or antigen-binding fragments thereof that bind to the alpha v beta 5 (αvβ5) integrin and uses thereof.

### BACKGROUND

Integrins are cell surface glycoprotein receptors that bind extracellular matrix proteins and mediate cell-cell and cell-extracellular matrix interactions, and cell-pathogen interactions. These receptors are composed of noncovalently associated alpha (α) and beta (β) chains that combine to give a variety of heterodimeric proteins with distinct cellular and adhesive specificities. These proteins can interact with cell surface ligands, transmembrane proteins, soluble proteases, pathogens, and growth factors.

The αvβ5 integrin is the only integrin that contains the β5 subunit. αv and β5 have both been sequenced and characterized (Hynes, 1992 supra and U.S. Pat. No. 5,527,679, respectively). αvβ5 recognizes the RGD peptide sequence and binds vitronectin (Hynes, Cell, 69:11-25 (1992). In addition, αvβ5 can activate TGF-β by a mechanism requiring an intact cytoskeleton and cell contraction. TGFβ1 is normally secreted as a complex composed of 3 proteins, including the bioactive peptide of TGFβ1, latency-associated peptide β1 (LAP-β1), and latent TGFβ (LTGFβ) binding protein 1 (LTBP-1). TGFβ1 forms a noncovalent complex with LAP-β1, which is called small latent complex (SLC), and in this configuration, TGFβ1 is unable to bind to its receptors. αvβ5 binds the latency-associated peptide β1 (LAP-β1) of the small latent complex (SLC) by recognizing an RGD motif and leads to activation of TGF-β. Furthermore, αvβ5 specifically regulates increases in vascular permeability induced by vascular endothelial growth factor (VEGF). However, αvβ5 regulation of vascular permeability is not restricted to VEGF-induced effects alone; blockade of αvβ5 prevents monolayer permeability induced by several different edemagenic agonists including TGF-β and thrombin.

The importance of integrins such as the αvβ5 integrin in biological processes is underscored by the pathological sequelae following integrin defects and from the often severe phenotypes of integrin subunit knockout animals.

### SUMMARY

This disclosure features antibodies and antigen-binding fragments thereof that specifically bind to αvβ5 and/or β5 and their use to treat, prevent, or reduce the symptoms or severity of αvβ5-mediated diseases or conditions.

In one aspect, the application discloses an isolated antibody or an antigen-binding fragment thereof that specifically binds to αvβ5 and/or β5, wherein the antibody or the antigen-binding fragment thereof comprises a heavy chain variable region that is at least 80% identical to the amino acid sequence set forth in any one of SEQ ID NOs:1 to 7. In certain embodiments, the antibody or an antigen-binding fragment thereof comprises a heavy chain variable region that is at least 81%, at least 82%, at least 83%, at least 84%, at least 85%, at least 86%, at least 87%, at least 88%, at least 89%, at least 90%, at least 95%, at least 96%, at least 97%, at least 98%, at least 99%, or 100% identical to the amino acid sequence set forth in any one of SEQ ID NOs:1 to 7.

In certain embodiments, the antibody or an antigen-binding fragment thereof that specifically binds to αvβ5 and/or β5, further comprises a light chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99%, or 100% identical to the amino acid sequence set forth in any one of SEQ ID NOs:8 to 12.

In some embodiments, the antibody or an antigen-binding fragment thereof that specifically binds to αvβ5 and/or β5, comprises a heavy chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:3 and comprises a light chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:10.

In other embodiments, the antibody or an antigen-binding fragment thereof that specifically binds to αvβ5 and/or β5, comprises a heavy chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:5 and comprises a light chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:8.

In yet other embodiments, the antibody or an antigen-binding fragment thereof that specifically binds to αvβ5 and/or β5, comprises a heavy chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:5 and comprises a light chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:9.

In certain embodiments, the antibody or an antigen-binding fragment thereof that specifically binds to αvβ5 and/or β5, comprises a heavy chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:5 and comprises a light chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:10.

In other embodiments, the antibody or an antigen-binding fragment thereof that specifically binds to αvβ5 and/or β5, comprises a heavy chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:6 and comprises a light chain variable region that is at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99% identical to the amino acid sequence set forth in SEQ ID NO:10.

The antibodies described above can, in some embodiments, comprise heavy chain complementarity determining regions (CDRs) 1, 2 and 3, wherein the heavy chain CDR 1 comprises/consists of the amino acid sequence set forth in SEQ ID NO:13, 58, 60, or 62, or the amino acid sequence set forth in SEQ ID NO:13, 58, 60, or 62 with a substitution at two or fewer amino acid positions, the heavy chain CDR 2 comprises/consists of the amino acid sequence set forth in SEQ ID NO:14, 59, 61, or 63, or the amino acid sequence set forth in SEQ ID NO:14, 59, 61, or 63 with a substitution at two or fewer amino acid positions, and the heavy chain CDR 3 comprises/consists of the amino acid sequence set forth in SEQ ID NO:15 or 64, or the amino acid sequence set forth in SEQ ID NO:15 or 64 with a substitution at two or fewer amino acid positions.

In one embodiment, antibodies described above comprise heavy chain CDRs 1, 2 and 3 wherein the heavy chain CDR 1 comprises/consists of the amino acid sequence set forth in SEQ ID NO:13, 58, 60, or 62, the heavy chain CDR 2 comprises/consists of the amino acid sequence set forth in SEQ ID NO:14, 59, 61, or 63, and the heavy chain CDR 3 comprises/consists of the amino acid sequence set forth in SEQ ID NO:15 or 64.

In another embodiment, antibodies described above comprise heavy chain CDRs 1, 2 and 3 wherein the heavy chain CDR 1 comprises/consists of the amino acid sequence set forth in SEQ ID NO:13, the heavy chain CDR 2 comprises/consists of the amino acid sequence set forth in SEQ ID NO:14, and the heavy chain CDR 3 comprises/consists of the amino acid sequence set forth in SEQ ID NO:15.

The antibodies described above can, in some embodiments, comprise light chain CDRs 1, 2 and 3 wherein the light chain CDR 1 comprises/consists of the amino acid sequence set forth in SEQ ID NO:16 or 65, or the amino acid sequence set forth in SEQ ID NO:16 or 65, with a substitution at two or fewer amino acid positions, the light chain CDR 2 comprises/consists of the amino acid sequence set forth in SEQ ID NO:17 or 66, or the amino acid sequence set forth in SEQ ID NO:17 or 66, with a substitution at two or fewer amino acid positions, and the light chain CDR 3 comprises/consists of the amino acid sequence set forth in SEQ ID NO:18 or 67, or the amino acid sequence set forth in SEQ ID NO:18 or 67 with a substitution at two or fewer amino acid positions.

In one embodiment, the antibodies described above comprise light chain CDRs 1, 2 and 3 wherein the light chain CDR 1 comprises/consists of the amino acid sequence set forth in SEQ ID NO:16 or 65, the light chain CDR 2 comprises/consists of the amino acid sequence set forth in SEQ ID NO:17 or 66, and the light chain CDR 3 comprises/consists of the amino acid sequence set forth in SEQ ID NO:18 or 67.

In another embodiment, the antibodies described above comprise light chain CDRs 1, 2 and 3 wherein the light chain CDR 1 comprises/consists of the amino acid sequence set forth in SEQ ID NO:16, the light chain CDR 2 comprises/consists of the amino acid sequence set forth in SEQ ID NO:17, and the light chain CDR 3 comprises/consists of the amino acid sequence set forth in SEQ ID NO:18.

The antibodies described above can, in some embodiments, comprise heavy chain complementarity determining regions (CDRs) 1, 2 and 3, wherein the heavy chain CDR 1 comprises/consists of the amino acid sequence set forth in SEQ ID NO:13, 58, 60, or 62, or the amino acid sequence set forth in SEQ ID NO:13, 58, 60, or 62 with a substitution at two or fewer amino acid positions, the heavy chain CDR 2 comprises/consists of the amino acid sequence set forth in SEQ ID NO:14, 59, 61, or 63, or the amino acid sequence set forth in SEQ ID NO:14, 59, 61, or 63 with a substitution at two or fewer amino acid positions, and the heavy chain CDR 3 comprises/consists of the amino acid sequence set forth in SEQ ID NO:15 or 64, or the amino acid sequence set forth in SEQ ID NO:15 or 64 with a substitution at two or fewer amino acid positions; and further comprise light chain CDRs 1, 2 and 3, wherein the light chain CDR 1 comprises/consists of the amino acid sequence set forth in SEQ ID NO:16 or 65, or the amino acid sequence set forth in SEQ ID NO:16 or 65, with a substitution at two or fewer amino acid positions, the light chain CDR 2 comprises/consists of the amino acid sequence set forth in SEQ ID NO:17 or 66, or the amino acid sequence set forth in SEQ ID NO:17 or 66, with a substitution at two or fewer amino acid positions, and the light chain CDR 3 comprises/consists of the amino acid sequence set forth in SEQ ID NO:18 or 67, or the amino acid sequence set forth in SEQ ID NO:18 or 67 with a substitution at two or fewer amino acid positions.

In one embodiment, the antibodies or antigen-binding fragments described herein comprise heavy chain CDRs 1, 2 and 3, wherein the heavy chain CDR 1 comprises/consists of the amino acid sequence set forth in SEQ ID NO:13, the heavy chain CDR 2 comprises/consists of the amino acid sequence set forth in SEQ ID NO:14, and the heavy chain CDR 3 comprises/consists of the amino acid sequence set forth in SEQ ID NO:15; and light chain CDRs 1, 2 and 3 wherein the light chain CDR 1 comprises/consists of the amino acid sequence set forth in SEQ ID NO:16, the light chain CDR 2 comprises/consists of the amino acid sequence set forth in SEQ ID NO:17, and the light chain CDR 3 comprises/consists of the amino acid sequence set forth in SEQ ID NO:18.

In some instances, the antibodies or antigen-binding fragment disclosed above comprises one to twenty-six (i.e., 1, 2, 3, 4, 5, 6, 7, 8, 9, 10, 11, 12, 13, 14, 15, 16, 17, 18, 19, 20, 21, 22, 23, 24, 25, or 26), of the following amino acids: (a) in the variable heavy chain: valine at position 4, glutamine at position 5, glutamine at position 6, glutamic acid at position 16, lysine at position 23, lysine at position 38, lysine at position 66, alanine at position 67, leucine at position 69, alanine at position 71, valine at position 72, threonine at position 73, proline or serine at position 75, and/or alanine at position 78; and (b) in the variable light chain: asparagine at position 1, leucine at position 11, threonine at position 12, valine at position 13, methionine at position 21, serine at position 22, serine at position 43, aspartic acid at position 60, threonine at position 63, valine at position 78, alanine at position 100, and/or leucine at position 104 (the numbering is according to Kabat).

In certain embodiments, the antibody or the antigen-binding fragment thereof comprises a heavy chain variable region that comprises the amino acid sequence set forth in SEQ ID NO:1. In other embodiments, the antibody or the antigen-binding fragment thereof comprises a heavy chain variable region that comprises the amino acid sequence set forth in SEQ ID NO:2. In yet other embodiments, the antibody or the antigen-binding fragment thereof comprises a heavy chain variable region that comprises the amino acid sequence set forth in SEQ ID NO:3. In other embodiments, the antibody or the antigen-binding fragment thereof comprises a heavy chain variable region that comprises the amino acid sequence set forth in SEQ ID NO:4. In some embodiments, the antibody or the antigen-binding fragment thereof comprises a heavy chain variable region that comprises the amino acid sequence set forth in SEQ ID NO:5. In certain embodiments, the antibody or the antigen-binding fragment thereof comprises a heavy chain variable region that comprises the amino acid sequence set forth in SEQ ID NO:6. In another embodiment, the antibody or the antigen-binding fragment thereof comprises a heavy chain variable region that comprises the amino acid sequence set forth in SEQ ID NO:7.

In certain embodiments, the antibody or the antigen-binding fragment thereof comprises a heavy chain variable region that comprises the amino acid sequence set forth in SEQ ID NO:3 and a light chain variable region that comprises the amino acid sequence set forth in SEQ ID NO:10. In other embodiments, the antibody or the antigen-binding fragment thereof comprises a heavy chain variable region that comprises the amino acid sequence set forth in SEQ ID NO:5 and a light chain variable region that comprises the amino acid sequence set forth in SEQ ID NO:8. In some embodiments, the antibody or the antigen-binding fragment thereof comprises a heavy chain variable region that comprises the amino acid sequence set forth in SEQ ID NO:5 and a light chain variable region that comprises the amino acid sequence set forth in SEQ ID NO:9. In some embodiments, the antibody or the antigen-binding fragment thereof comprises a heavy chain variable region that comprises the amino acid sequence set forth in SEQ ID NO:5 and a light chain variable region that comprises the amino acid sequence set forth in SEQ ID NO:10. In certain embodiments, the antibody or the antigen-binding fragment thereof comprises a heavy chain variable region that comprises the amino acid sequence set forth in SEQ ID NO:6 and a light chain variable region that comprises the amino acid sequence set forth in SEQ ID NO:10.

The above antibodies can have an isotype selected from the group consisting of IgG1, IgG2, IgG3, and IgG4. In certain embodiments, the antibodies have an IgG1 isotype. In other embodiments, the antibodies have an IgG4 isotype. In some instances, the antibody comprises a CH1 domain and a CH2 domain from an IgG antibody of the IgG4 isotype and a CH3 domain from an IgG antibody of the IgG1 isotype. In certain cases the antibody further comprises a S228P and/or an N297Q mutation (numbering according to Kabat).

In some embodiments, the antigen-binding fragments described above are selected from the group consisting of an Fab, an Fab′, an F(ab′)2, an Fv, a diabody, an scFv, and an sc(Fv)2.

In certain embodiments, the antibody comprises the heavy and light chains comprising/consisting of the amino acid sequences set forth in SEQ ID NO:69 and 70; SEQ ID NO:69 and 82; SEQ ID NO:80 and 82; or SEQ ID NO:81 and 70.

In some cases, the above antibodies or antigen-binding fragments thereof are conjugated to a substance selected from the group consisting of a toxin, a radionuclide, a fluorescent label, polyethylene glycol, a micro RNA, a drug, and a cytotoxic agent.

In some embodiments, the disclosure provides a pharmaceutical composition comprising the antibodies or the antigen-binding fragments thereof described above and a pharmaceutically acceptable carrier.

In one aspect, this disclosure provides a method of treating acute kidney injury in a human subject in need thereof, comprising administering to the human subject an antibody or the antigen-binding fragment thereof described herein.

In another aspect, this disclosure provides a method of treating acute lung injury in a human subject in need thereof, comprising administering to the human subject an antibody or the antigen-binding fragment thereof described herein.

In yet another aspect, this disclosure provides a method of treating stroke (cerebral hemorrhage) in a human subject in need thereof, comprising administering to the human subject an antibody or the antigen-binding fragment thereof described herein.

In a further aspect, this disclosure provides a method of treating lung fibrosis (e.g., IPF, UIP) in a human subject in need thereof, comprising administering to the human subject an antibody or the antigen-binding fragment thereof described herein.

In one aspect, this disclosure provides a method of treating pulmonary edema in a human subject in need thereof, comprising administering to the human subject an antibody or the antigen-binding fragment thereof described herein.

In another aspect, this disclosure provides a method of treating acute respiratory distress syndrome in a human subject in need thereof, comprising administering to the human subject an antibody or the antigen-binding fragment thereof described herein.

In another aspect, this disclosure provides a method of treating asthma in a human subject in need thereof, comprising administering to the human subject an antibody or the antigen-binding fragment thereof described herein.

In another aspect, this disclosure provides a method of treating sepsis in a human subject in need thereof, comprising administering to the human subject an antibody or the antigen-binding fragment thereof described herein.

In yet another aspect, this disclosure provides a method of treating cancer (e.g., pancreatic cancer, lung cancer, breast cancer, colorectal cancer, head and neck cancer, esophageal cancer, skin cancer, prostate cancer, cervical cancer, colon cancer, ovarian cancer, and endometrial cancer) in a human subject in need thereof, comprising administering to the human subject an antibody or the antigen-binding fragment thereof described herein.

In one aspect, this disclosure provides a method of inhibiting angiogenesis in a human subject in need thereof, comprising administering to the human subject an antibody or the antigen-binding fragment thereof described herein.

In another aspect, this disclosure provides a method of treating myocardial infraction in a human subject in need thereof, comprising administering to the human subject an antibody or the antigen-binding fragment thereof described herein.

In yet another aspect, this disclosure provides a method of treating a dyslipidemia in a human subject in need thereof, comprising administering to the human subject an antibody or the antigen-binding fragment thereof described herein.

In yet another aspect, this disclosure provides a method of treating obesity in a human subject in need thereof, comprising administering to the human subject an antibody or the antigen-binding fragment thereof described herein.

In a different aspect, the disclosure provides an isolated nucleic acid comprising a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a nucleotide sequence selected from the group consisting of SEQ ID NOs:95 to 102, 34, and 53 to 55. In certain embodiments, the disclosure encompasses the proteins encoded by these nucleic acids. In other embodiments, this application includes vectors comprising these nucleic acids. In certain embodiments, the vectors are transfected or transformed into host cells (e.g., CHO DG44i or CHO K1 GS).

In a different aspect, the disclosure provides an isolated nucleic acid comprising a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a nucleotide sequence selected from the group consisting of SEQ ID NOs: 1-7, 8-12, 69, 70, 80, 81, and 82. In certain embodiments, the disclosure encompasses the proteins encoded by these nucleic acids. In other embodiments, this application includes vectors comprising these nucleic acids. In certain embodiments, the vectors are transfected or transformed into host cells (e.g., CHO DG44i or CHO K1 GS).

In another aspect, this disclosure provides a method of preparing a humanized antibody. The method involves culturing a host cell comprising recombinant vectors comprising the nucleic acids disclosed above. For example, the nucleic acid sequences include those set forth in SEQ ID NOs:99 and 53; those set forth in SEQ ID NOs:99 and 102; those set forth in SEQ ID NOs:97 and 102; those set forth in SEQ ID NOs:100 and 53; and those set forth in SEQ ID NOs:99 and 34. The culturing is performed under conditions appropriate for expression of the antibody (e.g., a humanized antibody). The antibody chains are expressed and the antibody is produced. In certain embodiments, the method involves isolating the antibody. In some embodiments, the host cell is a CHO cell (e.g., CHO DG44i or CHO K1 GS).

In another aspect, this disclosure provides an antibody or antigen-binding fragment thereof that specifically binds to αvβ5 and/or β5 that is suitable for use in the treatment of a human subject and for large scale manufacture and storage. In some embodiments, the antibody or antigen-binding fragment thereof shows improved binding and inhibition properties compared with a murine αvβ5 antibody (e.g., ALULA, mouse chimeric ALULA) and/or other humanized anti-αvβ5 antibodies. Such improved binding and inhibition properties can be tested as shown in Example 6. In addition, the anti-αvβ5 antibody in some embodiments shows reduced fragmentation and maintains a higher level of monomer integrity at low pH than many other humanized anti-αvβ5 antibodies. Furthermore, the anti-αvβ5 antibody in some embodiments shows conformational stability that is comparable with other humanized anti-αvβ5 antibodies. Also, the anti-αvβ5 antibody or antigen-binding fragment thereof in some embodiments shows greater resistance to aggregation under accelerated stress condition such as elevated temperature, freeze-thaw, and/or agitation, compared with other humanized anti-αvβ5 antibodies. In one embodiment, the anti-αvβ5 antibody or antigen-binding fragment thereof of this aspect comprises a VH comprising the amino acid sequence set forth in SEQ ID NO:5. In one embodiment, the anti-αvβ5 antibody or antigen-binding fragment thereof of this aspect comprises a VH comprising the amino acid sequence set forth in SEQ ID NO:5 and a VL comprising the amino acid sequence set forth in SEQ ID NO:10.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a graph showing that reducing effector function of a humanized anti-αvβ5 antibody does not affect its efficacy as determined in a rat ischemia-reperfusion model.

FIG. 2 is an alignment of the variable heavy chain (VH) amino acid sequences of seven humanized ALULA VH regions with the VH region of ALULA (i.e., the murine anti-αvβ5 antibody). The mutations in the humanized versions VH1 to VH6 compared to the humanized VH0 CDR graft are shown in bold, lower case font. The amino acids that differ between the ALULA VH region and the humanized ALULA VH CDR graft are highlighted in gray. The CDR regions (VHCDR1, VHCDR2, and VHCDR3) are underlined.

FIG. 3 is an alignment of the variable light chain (VL) amino acid sequences of five humanized ALULA VL regions with the VL region of ALULA. The mutations in the humanized versions VL1 to VL4 compared to the humanized VL0 CDR graft are shown in bold, lower case font. The amino acids that differ between the ALULA VL region and the humanized ALULA VL CDR graft are highlighted in gray. The CDR regions (VLCDR1, VLCDR2, and VLCDR3) are underlined.

FIG. 4 is a graphical representation of experiments performed using the humanized ALULA antibodies described herein to assess their binding to soluble purified human αvβ5 protein in a competition ELISA with ALULA.

FIG. 5 is a graphical representation of the results of experiments performed to determine the efficacy of a humanized anti-αvβ5 antibody, H4/L2 (comprised of SEQ ID NOs.: 69 and 70), in the prevention of renal ischemia in the rat unilateral ischemic clamp model.

FIG. 6A is a graphical depiction of aggregation levels (% High Molecular Weight (HMW) species) by size exclusion chromatography (SEC) in the indicated antibody constructs following 2 weeks at 40° C./75% RH (relative humidity) conditions.

FIG. 6B is a graphical depiction of Low Molecular Weight (LMW) protein fragments as determined by SEC in the indicated antibody constructs following 2 weeks of 40° C./75% RH conditions.

FIG. 6C is a graphical depiction of the percent monomer by GXII LabChip in the indicated antibody constructs following 2 weeks of 40° C./75% RH conditions.

FIG. 7A is a bar graph showing the increase in aggregate (% High Molecular Weight species) by SEC in the indicated antibody constructs following 2 weeks at 40° C./75% RH conditions and at elevated concentration.

FIG. 7B is a bar graph that depicts the increase in aggregate (% HMW) by SEC in the indicated antibody constructs at elevated concentration following multiple freeze-thaw cycles.

FIG. 7C is a bar graph showing the increase in aggregate (% HMW) by SEC in the indicated antibody constructs following multiday room temperature agitation at elevated concentration.

### DETAILED DESCRIPTION

This disclosure features antibodies and antigen-binding fragments that specifically bind the αvβ5 integrin and/or the β5 subunit of this integrin. The αvβ5 integrin recognizes the RGD peptide sequence in a wide variety of ligands. For example, the antibodies and antigen-binding fragments thereof described herein block the interaction between αvβ5 and its ligands such as vitronectin. In addition, the antibodies and antigen-binding fragments thereof described herein can also block the interaction between αvβ5 and one or more of its other ligands such as fibronectin, osteopontin, tenascin c, cytotactin, fibrinogen, laminin, matrix metalloproteinase-2, osteomodulin, prothrombin, thrombospondin, Von Willebrand factor (vWF), and adenovirus penton base. The antibodies or antigen-binding fragments thereof described herein can also inhibit the interaction between αvβ5 and LAP of TGF-β; inhibit the activation of TGF-β; bind rat, mouse, cynomolgus, and human αvβ5; bind to αvβ5 expressed on the cell surface (e.g., of BaF3) with a K_{D} of 0.01 to 2 nM (e.g., 0.02, 0.04, 0.06, 0.08, 1.0, 1.2, 1.4, 1.6, or 1.8 nM); bind to recombinant αvβ5 with an apparent affinity of about 5 pM to about 500 pM (e.g., 25 pM to 500 pM, 25 pM to 150 pM, 50 pM to 100 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 125 pM, or 150 pM); inhibit αvβ5 binding to vitronectin in an ELISA assay using purified proteins with an IC₅₀ value of about 1 nM to about 5 nM (e.g., 1 nM, 1.5 nM, 2.0 nM, 2.2 nM, 2.3 nM, 2.4 nM, 2.5 nM, 2.6 nM, 2.7 nM, 2.8 nM, 2.9 nM, 3.0 nM, 3.2 nM, 3.5 nM, 3.8 nM, 4.0 nM, 4.5 nM, 5.0 nM); and/or inhibit αvβ5 binding to vitronectin in a cell adhesion assay with an IC₅₀ of 0.1 to 2 nM (e.g., 0.2, 0.3, 0.4, 0.5, 0.6, 0.7, 0.8, 0.9, 1.0, 1.1, 1.2, 1.3, 1.4, 1.5, 1.6, 1.7, or 1.8 nM). The antibodies and antigen-binding fragments described herein block αvβ5 and thereby inhibit vascular permeability in response to inflammation and injury; inhibit endothelial migration; and inhibit TGF-β activation and fibrosis. These antibodies and antigen-binding fragments are useful in treatment of a wide range of disorders such as acute kidney injury, acute lung injury, stroke (cerebral hemorrhage), acute respiratory distress syndrome, pulmonary edema, lung fibrosis (e.g., idiopathic pulmonary fibrosis (IPF), usual interstitial pneumonia (UIP)), sepsis, myocardial infarction, cancer (e.g., pancreatic cancer, lung cancer, breast cancer, colorectal cancer, head and neck cancer, esophageal cancer, skin cancer, prostate cancer, cervical cancer, colon cancer, ovarian cancer, and endometrial cancer), and ocular neovascularization disease. In addition, the antibodies or the antigen-binding fragments of this disclosure can be used to treat or prevent a pathogenic (e.g., viral) infection, where the pathogenic infection proceeds, at least in part, by an interaction between a pathogen's RGD-containing protein and αvβ5.

αv:

The amino acid sequence of the human αv protein (Uniprot Accession No. P06756-1) is shown below:

(SEQ ID NO: 50)

MAFPPRRRLR LGPRGLPLLL SGLLLPLCRA FNLDVDSPAE
YSGPEGSYFG FAVDFFVPSA SSRMFLLVGA PKANTTQPGI
VEGGQVLKCD WSSTRRCQPI EFDATGNRDY AKDDPLEFKS
HQWFGASVRS KQDKILACAP LYHWRTEMKQ EREPVGTCFL
QDGTKTVEYA PCRSQDIDAD GQGFCQGGFS IDFTKADRVL
LGGPGSFYWQ GQLISDQVAE IVSKYDPNVY SIKYNNQLAT
RTAQAIFDDS YLGYSVAVGD FNGDGIDDFV SGVPRAARTL
GMVYIYDGKN MSSLYNFTGE QMAAYFGFSV AATDINGDDY
ADVFIGAPLF MDRGSDGKLQ EVGQVSVSLQ RASGDFQTTK
LNGFEVFARF GSAIAPLGDL DQDGFNDIAI AAPYGGEDKK
GIVYIFNGRS TGLNAVPSQI LEGQWAARSM PPSFGYSMKG
ATDIDKNGYP DLIVGAFGVD RAILYRARPV ITVNAGLEVY
PSILNQDNKT CSLPGTALKV SCFNVRFCLK ADGKGVLPRK
LNFQVELLLD KLKQKGAIRR ALFLYSRSPS HSKNMTISRG
GLMQCEELIA YLRDESEFRD KLTPITIFME YRLDYRTAAD
TTGLQPILNQ FTPANISRQA HILLDCGEDN VCKPKLEVSV
DSDQKKIYIG DDNPLTLIVK AQNQGEGAYE AELIVSIPLQ
ADFIGVVRNN EALARLSCAF KTENQTRQVV CDLGNPMKAG
TQLLAGLRFS VHQQSEMDTS VKFDLQIQSS NLFDKVSPVV
SHKVDLAVLA AVEIRGVSSP DHVFLPIPNW EHKENPETEE
DVGPVVQHIY ELRNNGPSSF SKAMLHLQWP YKYNNNTLLY
ILHYDIDGPM NCTSDMEINP LRIKISSLQT TEKNDTVAGQ
GERDHLITKR DLALSEGDIH TLGCGVAQCL KIVCQVGRLD
RGKSAILYVK SLLWTETFMN KENQNHSYSL KSSASFNVIE
FPYKNLPIED ITNSTLVTTN VTWGIQPAPM PVPVWVIILA
VLAGLLLLAV LVFVMYRMGF FKRVRPPQEE QEREQLQPHE
NGEGNSET

β5:

The amino acid sequence of the human β5 protein (Genbank® Accession No. NP_002204.2) is shown below:

(SEQ ID NO: 51)

MPRAPAPLYACLLGLCALLPRLAGLNICTSGSATSCEECLLIHPKCAWCS
KEDFGSPRSITSRCDLRANLVKNGCGGEIESPASSFHVLRSLPLSSKGSG
SAGWDVIQMTPQEIAVNLRPGDKTTFQLQVRQVEDYPVDLYYLMDLSLSM
KDDLDNIRSLGTKLAEEMRKLTSNFRLGFGSFVDKDISPFSYTAPRYQTN
PCIGYKLFPNCVPSFGFRHLLPLTDRVDSFNEEVRKQRVSRNRDAPEGGF
DAVLQAAVCKEKIGWRKDALHLLVFTTDDVPHIALDGKLGGLVQPHDGQC
HLNEANEYTASNQMDYPSLALLGEKLAENNINLIFAVTKNHYMLYKNFTA
LIPGTTVEILDGDSKNIIQLIINAYNSIRSKVELSVWDQPEDLNLFFTAT
CQDGVSYPGQRKCEGLKIGDTASFEVSLEARSCPSRHTEHVFALRPVGFR
DSLEVGVTYNCTCGCSVGLEPNSARCNGSGTYVCGLCECSPGYLGTRCEC
QDGENQSVYQNLCREAEGKPLCSGRGDCSCNQCSCFESEFGKIYGPFCEC
DNFSCARNKGVLCSGHGECHCGECKCHAGYIGDNCNCSTDISTCRGRDGQ
ICSERGHCLCGQCQCTEPGAFGEMCEKCPTCPDACSTKRDCVECLLLHSG
KPDNQTCHSLCRDEVITWVDTIVKDDQEAVLCFYKTAKDCVMMFTYVELP
SGKSNLTVLREPECGNTPNAMTILLAVVGSILLVGLALLAIWKLLVTIHD
RREFAKFQSERSRARYEMASNPLYRKPISTHTVDFTFNKFNKSYNGTVD

The amino acid sequence of the murine β5 protein (Genbank® Accession No. NP_001139356.1) is shown below:

(SEQ ID NO: 52)

MPRVPATLYACLLGLCALVPRLAGLNICTSGSATSCEECLLIHPKCAWCS
KEYFGNPRSITSRCDLKANLIRNGCEGEIESPASSTHVLRNLPLSSKGSS
ATGSDVIQMTPQEIAVSLRPGEQTTFQLQVRQVEDYPVDLYYLMDLSLSM
KDDLENIRSLGTKLAEEMRKLTSNFRLGFGSFVDKDISPFSYTAPRYQTN
PCIGYKLFPNCVPSFGFRHLLPLTDRVDSFNEEVRKQRVSRNRDAPEGGF
DAVLQAAVCKEKIGWRKDALHLLVFTTDDVPHIALDGKLGGLVQPHDGQC
HLNEANEYTASNQMDYPSLALLGEKLAENNINLIFAVTKNHYMLYKNFTA
LIPGTTVEILHGDSKNIIQLIINAYSSIRAKVELSVWDQPEDLNLFFTAT
CQDGISYPGQRKCEGLKIGDTASFEVSVEARSCPGRQAAQSFTLRPVGFR
DSLQVEVAYNCTCGCSTGLEPNSARCSGNGTYTCGLCECDPGYLGTRCEC
QEGENQSGYQNLCREAEGKPLCSGRGECSCNQCSCFESEFGRIYGPFCEC
DSFSCARNKGVLCSGHGECHCGECKCHAGYIGDNCNCSTDVSTCRAKDGQ
ICSDRGRCVCGQCQCTEPGAFGETCEKCPTCPDACSSKRDCVECLLLHQG
KPDNQTCHHQCKDEVITWVDTIVKDDQEAVLCFYKTAKDCVMMFSYTELP
NGRSNLTVLREPECGSAPNAMTILLAVVGSILLIGMALLAIWKLLVTIHD
RREFAKFQSERSRARYEMASNPLYRKPISTHTVDFAFNKFNKSYNGSVD

### Anti-αvβ5 Antibodies

This disclosure includes antibodies and antigen-binding fragments that specifically bind to αvβ5 and/or the β5 subunit. The antibodies disclosed herein are based on the complementarity determining regions (CDRs) of the ALULA murine antibody that is produced by the hybridoma deposited at the ATCC on Feb. 13, 2004, with the accession number PTA-5817. The mature VH and VL sequences of the murine anti-αvβ5 antibody, ALULA, are provided below (the CDRs based on the Kabat definition are underlined).

```
ALULA VH:
(SEQ ID NO: 71)
                                                            VHCDR1 VHCDR2
EVQVQQSGTVLARPGASVKMSCKASGYTFTSYWMHWVKQRPGQGLEWIGAIYPGNSDTSYNQKFKGKAKL
                                                            VHCDR3
TAVTSPNTAYMELSSLTNEDSAVYYCTTTTYGYDWFAYWGQGTLVTVSA
ALULA VL:
(SEQ ID NO: 72)
                                                VLCDR1 VLCDR2
NIMMTQSPSSLTVSAGEKVTMSCKSSQSVLYSSNQKNYLAWYQQKPGQSPKLLIYWASTRESGVPDRFTG
                                                VLCDR3
SGSGTDFTLTISSVQAEDLAVYYCHQYLSSLTFGAGTKLELK
```

Example 2 discloses seven exemplary humanized heavy chain variable regions, VH0, VH1, VH2, VH3, VH4, VH5, and VH6 having the amino acid sequences set forth in SEQ ID NOs:1, 2, 3, 4, 5, 6, and 7 respectively, and five exemplary humanized light chain variable regions, VL0, VL1, VL2, VL3, and VL4, having the amino acid sequences set forth in SEQ ID NOs:8, 9, 10, 11 and 12, respectively. Each of the VH chains can pair with any of the VL chains: i.e., VH0 can pair with VL0, VL1, VL2, VL3, or VL4; VH1 can pair with VL0, VL1, VL2, VL3, or VL4; VH2 can pair with VL0, VL1, VL2, VL3, or VL4; VH3 can pair with VL0, VL1, VL2, VL3, or VL4; VH4 can pair with VL0, VL1, VL2, VL3, or VL4; VH5 can pair with VL0, VL1, VL2, VL3, or VL4; and VH6 can pair with VL0, VL1, VL2, VL3, or VL4. Thus, the heavy chain variable region and light chain variable regions disclosed in Example 2 can form 35 different VH-VL pairs. All of these antibodies are considered part of this disclosure. These antibodies can comprise a kappa light chain constant region. In one embodiment, the light chain constant region has the following amino acid sequence:

(SEQ ID NO: 56)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

These antibodies can also comprise a heavy chain constant region. In one embodiment, the heavy chain constant region has the following sequence:

(SEQ ID NO: 57)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFQSTYRVVSVLTVLHQDWLNGKEYK
CKVSNKGLPSSIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG

The amino acid sequences of the heavy and light chain CDRs 1, 2, and 3, as well as the framework regions (FRs) 1, 2, 3, 4 of the seven heavy chain variable regions and the five light chain variable regions of the exemplary humanized anti-αvβ5 antibodies described in Example 2 are provided below. The CDRs are based upon the Kabat numbering system.

| Domain | SEQ ID | Sequence |
|---|---|---|
| VH CDR1 | 13 | SYWMH |
| VH CDR2 | 14 | AIYPGNSDTSYNQKFKG |
| VH CDR3 | 15 | TTYGYDWFAY |
| VL CDR1 | 16 | KSSQSVLYSSNQKNYLA |
| VL CDR2 | 17 | WASTRES |
| VL CDR3 | 18 | HQYLSSLT |
| VH0 FR1 | 19 | EVQLVESGGGLVKPGGSLRLSCAAS |
| VH0 FR2 | 20 | WVRQAPGKGLEWVG |
| VH0 FR3 | 21 | RFTISRDDSKNTLYLQMNSLKTEDTAVYYCTT |
| VH0 FR4 | 22 | WGQGTLVTVSS |
| VH1 FR1 | 23 | EVQVVESGGGLVKPGGSLRLSCAAS |
| VH1 FR2 | 20 | WVRQAPGKGLEWVG |
| VH1 FR3 | 24 | RFTISADTSKNTLYLQMNSLKTEDTAVYYCTT |
| VH1 FR4 | 22 | WGQGTLVTVSS |
| VH2 FR1 | 25 | EVQVVQSGGGLVKPGESLRLSCAAS |
| VH2 FR2 | 26 | WVKQAPGKGLEWVG |
| VH2 FR3 | 27 | KFTISADTSSNTAYLQMNSLKTEDTAVYYCTT |
| VH2 FR4 | 22 | WGQGTLVTVSS |
| VH3 FR1 | 28 | EVQVVESGGGLVKPGGSLRLSCKAS |
| VH3 FR2 | 26 | WVKQAPGKGLEWVG |
| VH3 FR3 | 29 | KFTLSAVTSSNTAYLQMNSLKTEDTAVYYCTT |
| VH3 FR4 | 22 | WGQGTLVTVSS |
| VH4 FR1 | 30 | EVQVVQSGGGLVKPGESLRLSCKAS |
| VH4 FR2 | 26 | WVKQAPGKGLEWVG |
| VH4 FR3 | 31 | KFTISADTSPNTAYLQMNSLKTEDTAVYYCTT |
| VH4 FR4 | 22 | WGQGTLVTVSS |
| VH5 FR1 | 32 | EVQVQQSGGGLVKPGGSLRLSCKAS |
| VH5 FR2 | 26 | WVKQAPGKGLEWVG |
| VH5 FR3 | 33 | KATLSAVTSPNTAYLQMNSLKTEDTAVYYCTT |
| VH5 FR4 | 22 | WGQGTLVTVSS |
| VH6 FR1 | 23 | EVQVVESGGGLVKPGGSLRLSCAAS |
| VH6 FR2 | 20 | WVRQAPGKGLEWVG |
| VH6 FR3 | 21 | RFTISADTSKNTLYLQMNSLKTEDTAVYYCTT |
| VH6 FR4 | 22 | WGQGTLVTVSS |
| VH6 CDR2 | 35 | RIKSKTDGGTTDYAAPVKG |
| VL0 FR1 | 36 | DIQMTQSPSSVSASVGDRVTITC |
| VL0 FR2 | 37 | WYQQKPGKAPKLLIY |
| VL0 FR3 | 38 | GVPSRFSGSGSGTDFTLTISSLQPEDFATYYC |
| VL0 FR4 | 39 | FGQGTKVEIK |
| VL1 FR1 | 40 | DIQMTQSPSSLSASVGDRVTMTC |
| VL1 FR2 | 37 | WYQQKPGKAPKLLIY |
| VL1 FR3 | 41 | GVPDRFSGSGSGTDFTLTISSLQPEDFATYYC |
| VL1 FR4 | 42 | FGQGTKLEIK |
| VL2 FR1 | 43 | NIQMTQSPSSLSASVGDRVTMSC |
| VL2 FR2 | 44 | WYQQKPGKSPKLLIY |
| VL2 FR3 | 45 | GVPDRFTGSGSGTDFTLTISSLQPEDFATYYC |
| VL2 FR4 | 42 | FGQGTKLEIK |
| VL3 FR1 | 46 | DIQMTQSPSSLTVSVGDRVTMSC |
| VL3 FR2 | 44 | WYQQKPGKSPKLLIY |
| VL3 FR3 | 47 | GVPDRFTGSGSGTDFTLTISSVQPEDFATYYC |
| VL3 FR4 | 48 | FGAGTKLEIK |
| VL4 FR1 | 40 | DIQMTQSPSSLSASVGDRVTMTC |
| VL4 FR2 | 37 | WYQQKPGKAPKLLIY |
| VL4 FR3 | 38 | GVPSRFSGSGSGTDFTLTISSLQPEDFATYYC |
| VL4 FR4 | 42 | FGQGTKLEIK |
| VL4 CDR2 | 49 | AASSLQS |

This application also discloses “alternate CDRs” of ALULA that can be used instead of the Kabat CDRs in the antibodies of this disclosure. By “alternate” CDRs are meant CDRs (CDR1, CDR2, and CDR3) defined according to a definition other than Kabat (e.g., Chothia from AbYsis, enhanced Chothia/AbM CDR, or the contact definitions). These alternate CDRs can be obtained, e.g., by using the AbYsis database (www.bioinf.org.uk/abysis/sequence_input/key_annotation/key_annotation.cgi). The amino acid sequences of exemplary “alternate” CDRs 1, 2, and 3 of the heavy chain variable region and the light chain variable region of ALULA are compared with the CDRs defined according to Kabat in the Table below.

| Domain | Kabat | Chothia from AbYsis | Enhanced Chothia/AbM | Contact |
|---|---|---|---|---|
| VH CDR1 | SYWMH | GYTFTSY | GYTFTSYWMH | TSYWMH |
| | (SEQ ID NO: 13) | (SEQ ID NO: 58) | (SEQ ID NO: 60) | (SEQ ID NO: 62) |
| VH CDR2 | AIYPGNSDTSYNQKFKG | YPGNSD | AIYPGNSDTS | WIGAIYPGNSDTS |
| | (SEQ ID NO: 14) | (SEQ ID NO: 59) | (SEQ ID NO: 61) | (SEQ ID NO: 63) |
| VH CDR3 | TTYGYDWFAY | TTYGYDWFAY | TTYGYDWFAY | TTTTYGYDWFA |
| | (SEQ ID NO: 15) | (SEQ ID NO: 15) | (SEQ ID NO: 15) | (SEQ ID NO: 64) |
| VL CDR1 | KSSQSVLYSSNQKNYLA | KSSQSVLYSSNQKNYLA | KSSQSVLYSSNQKNYLA | LYSSNQKNYLAWY |
| | (SEQ ID NO: 16) | (SEQ ID NO: 16) | (SEQ ID NO: 16) | (SEQ ID NO: 65) |
| VL CDR2 | WASTRES | WASTRES | WASTRES | LLIYWASTRE |
| | (SEQ ID NO: 17) | (SEQ ID NO: 17) | (SEQ ID NO: 17) | (SEQ ID NO: 66) |
| VL CDR3 | HQYLSSLT | HQYLSSLT | HQYLSSLT | HQYLSSL |
| | (SEQ ID NO: 18) | (SEQ ID NO: 18) | (SEQ ID NO: 18) | (SEQ ID NO: 67) |

In some instances, the anti-αvβ5 antibodies or antigen binding fragments thereof comprise a VH or heavy chain comprising H-CDR1, H-CDR2, and H-CDR3 comprising/consisting of the amino acid sequences set forth in SEQ ID NOs:13, 14, and 15, respectively. In other instances, the anti-αvβ5 antibodies or antigen binding fragments thereof comprise a VH or heavy chain comprising H-CDR1, H-CDR2, and H-CDR3 comprising/consisting of the amino acid sequences set forth in SEQ ID NOs:58, 59, and 15, respectively. In yet other instances, the anti-αvβ5 antibodies or antigen binding fragments thereof comprise a VH or heavy chain comprising H-CDR1, H-CDR2, and H-CDR3 comprising/consisting of the amino acid sequences set forth in SEQ ID NOs:60, 61, and 15, respectively. In certain embodiments, all of the above-described anti-αvβ5 antibodies or antigen binding fragments thereof comprise a VL or light chain comprising L-CDR1, L-CDR2, and L-CDR3 comprising/consisting of the amino acid sequences set forth in SEQ ID NOs:16, 17, and 18, respectively. The above described anti-αvβ5 antibodies can have a light chain constant region comprising/consisting of the sequence set forth in SEQ ID NO:56 and/or a heavy chain constant region comprising/consisting of the sequence set forth in SEQ ID NO:57.

In some instances, the anti-αvβ5 antibodies or antigen binding fragments thereof comprise a VH or heavy chain comprising H-CDR1, H-CDR2, and H-CDR3 comprising/consisting of the amino acid sequences set forth in SEQ ID NOs:62, 63, and 64, respectively. In certain embodiments, these anti-αvβ5 antibodies or antigen binding fragments thereof comprise a VL or light chain comprising L-CDR1, L-CDR2, and L-CDR3 comprising/consisting of the amino acid sequences set forth in SEQ ID NOs:65, 66, and 67, respectively. The above described anti-αvβ5 antibodies can have a light chain constant region comprising/consisting of the sequence set forth in SEQ ID NO:56 and/or a heavy chain constant region comprising/consisting of the sequence set forth in SEQ ID NO:57.

The anti-αvβ5 antibodies or antigen-binding fragments thereof described herein can include heavy chain framework regions H-FR1, H-FR2, H-FR3, and H-FR4, wherein H-FR1 has an amino acid sequence selected from the group consisting of SEQ ID NOs:19, 23, 25, 28, 30, and 32; H-FR2 has an amino acid sequence selected from SEQ ID NOs:20 and 26; H-FR3 has an amino acid sequence selected from the group consisting of SEQ ID NOs:21, 24, 27, 29, 31, and 33; and H-FR4 has an amino acid sequence set forth in SEQ ID NO.:22. In certain instances, anti-αvβ5 antibodies described herein can include light chain framework regions L-FR1, L-FR2, L-FR3, and L-FR4, wherein L-FR1 has an amino acid sequence selected from the group consisting of SEQ ID NOs:36, 40, 43, and 46; L-FR2 has an amino acid sequence selected from SEQ ID NOs:37 and 44; L-FR3 has an amino acid sequence selected from the group consisting of SEQ ID NOs:38, 41, 45, and 47; and L-FR4 has an amino acid sequence set forth in SEQ ID NO.:39, 42, or 48. In a specific embodiment, the anti-αvβ5 antibody or antigen-binding fragments thereof comprises (i) heavy chain framework regions H-FR1, H-FR2, H-FR3, and H-FR4, wherein H-FR1 has an amino acid sequence set forth in SEQ ID NO:36; H-FR2 has an amino acid sequence set forth in SEQ ID NO:26; H-FR3 has an amino acid sequence set forth in SEQ ID NO:31; and H-FR4 has an amino acid sequence set forth in SEQ ID NO:22; and (ii) light chain framework regions L-FR1, L-FR2, L-FR3, and L-FR4, wherein L-FR1 has an amino acid sequence set forth in SEQ ID NO:43; L-FR2 has an amino acid sequence set forth in SEQ ID NO:44; L-FR3 has an amino acid sequence set forth in SEQ ID NO:45; and L-FR4 has an amino acid sequence set forth in SEQ ID NO:42.

This disclosure also includes antibodies or antigen-binding fragments thereof that specifically bind αvβ5 and/or β5 that have heavy chain variable regions that are: at least 75%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequences set forth in any one of SEQ ID NO:1 to SEQ ID NO:7. In some embodiments, these antibodies or antigen-binding fragments thereof have the VH CDR1 of ALULA with two or fewer substitutions, the VHCDR2 of ALULA with two or fewer substitutions, and the VH CDR3 of ALULA with two or fewer substitutions. In other embodiments, these antibodies or antigen-binding fragments thereof have the VHCDR1 of ALULA, the VHCDR2 of ALULA with three or fewer substitutions, and the VHCDR3 of ALULA. In other embodiments, these antibodies or antigen-binding fragments thereof have the VHCDR1 of ALULA, the VHCDR2 of ALULA with one substitution, and the VHCDR3 of ALULA. In a specific embodiment, these antibodies or antigen-binding fragments thereof have the VHCDR1 of ALULA, the VHCDR2 of ALULA, and the VHCDR3 of ALULA. The above antibodies or antigen-binding fragments thereof can inhibit the interaction between αvβ5 and vitronectin; inhibit the interaction between αvβ5 and LAP of TGF-β; and/or inhibit the activation of TGF-β. The CDRs referenced above can be the Kabat CDRs or alternate CDRs.

In some embodiments, these anti-αvβ5 antibodies or antigen-binding fragments thereof further include a light chain variable region that is at least 75%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences set forth in SEQ ID NO:8 to SEQ ID NO:12. In some embodiments, these antibodies or antigen-binding fragments thereof have the VLCDR1 of ALULA with two or fewer substitutions, the VLCDR2 of ALULA with two or fewer substitutions, and the VLCDR3 of ALULA with two or fewer substitutions. In some embodiments, these antibodies or antigen-binding fragments thereof have the VLCDR1 of ALULA, the VLCDR2 of ALULA with two or fewer substitutions, and the VLCDR3 of ALULA. In some embodiments, these antibodies or antigen-binding fragments thereof have the VLCDR1 of ALULA, the VLCDR2 of ALULA, and the VLCDR3 of ALULA. In yet other embodiments, these antibodies or antigen-binding fragments thereof have the VHCDR1 of ALULA, the VHCDR2 of ALULA with three or fewer substitutions, and the VHCDR3 of ALULA; and the VLCDR1 of ALULA, the VLCDR2 of ALULA with two or fewer substitutions, and the VLCDR3 of ALULA. In a specific instance, these antibodies or antigen-binding fragments thereof include all of the heavy and light CDRs of ALULA. The CDRs referenced above can be the Kabat CDRs or alternate CDRs. The above antibodies or antigen-binding fragments thereof can inhibit the interaction between αvβ5 and vitronectin; can inhibit the interaction between αvβ5 and LAP of TGF-β; can inhibit the activation of TGF-β; can bind rat, mouse, cynomolgus, and human αvβ5; can bind to αvβ5 recombinantly expressed on the cell surface (e.g., of BaF3) with a K_{D} of 0.01 to 2 nM (e.g., 0.02, 0.04, 0.06, 0.08, 1.0, 1.2, 1.4, 1.6, 1.8 nM); bind to recombinant αvβ5 with an apparent affinity of about 5 pM to about 500 pM (e.g., 25 pM to 500 pM, 25 pM to 150 pM, 50 pM to 100 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 125 pM, or 150 pM); inhibit αvβ5 binding to vitronectin in an ELISA assay using purified proteins with an IC₅₀ value of about 1 nM to about 5 nM (e.g., 1 nM, 1.5 nM, 2.0 nM, 2.2 nM, 2.3 nM, 2.4 nM, 2.5 nM, 2.6 nM, 2.7 nM, 2.8 nM, 2.9 nM, 3.0 nM, 3.2 nM, 3.5 nM, 3.8 nM, 4.0 nM, 4.5 nM, or 5.0 nM); and/or can inhibit αvβ5 binding to vitronectin in a cell adhesion assay with an IC₅₀ of 0.1 to 2 nM (e.g., 0.2, 0.3, 0.4, 0.5, 0.6, 0.7, 0.8, 0.9, 1.0, 1.1, 1.2, 1.3, 1.4, 1.5, 1.6, 1.7, or 1.8 nM).

This disclosure also includes antibodies or antigen-binding fragments thereof that specifically bind αvβ5 and/or β5 that have four or fewer (e.g., four, three or fewer, three, two or fewer, two, or one) amino acid substitutions in one, two, three, or all four of the framework regions, and/or four or fewer (e.g., four, three or fewer, two or fewer, or one) amino acid substitutions in one, two, or all three CDRs (or alternate CDRs), of the heavy chain variable region comprising the amino acid sequences set forth in SEQ ID NOs:1, 2, 3, 4, 5, 6, or 7. The application also includes antibodies or antigen-binding fragments thereof that have four or fewer (e.g., four, three or fewer, three, two or fewer, two, or one) amino acid substitutions in one, two, three, or all four of the framework regions, and/or four or fewer (e.g., four, three or fewer, three, two or fewer, two, or one) amino acid substitutions in one, two, or all three CDRs (or alternate CDRs), of the light chain variable regions comprising the amino acid sequences set forth in SEQ ID NOs:8, 9, 10, 11, or 12. In certain embodiments, the humanized antibodies of this disclosure include antibodies that specifically bind αvβ5 and/or β5 that have four or fewer (e.g., four, three or fewer, three, two or fewer, two, or one) amino acid substitutions in one, two, three, or four of the framework regions, and/or four or fewer (e.g., four, three or fewer, three, two or fewer, two, or one) amino acid substitutions in one, two, or three CDRs (or alternate CDRs), of the heavy chain variable region comprising the amino acid sequences set forth in SEQ ID NOs:1, 2, 3, 4, 5, 6, or 7; and four or fewer (e.g., four, three or fewer, three, two or fewer, two, or one) amino acid substitutions in one, two, three, or four of the framework regions, and/or four or fewer (e.g., four, three or fewer, three, two or fewer, two, or one) amino acid substitutions in one, two, or three CDRs (or alternate CDRs), of the light chain variable regions comprising the amino acid sequences set forth in SEQ ID NOs:8, 9, 10, 11, or 12. In some embodiments, the amino acid substitutions are conservative amino acid substitutions. The above antibodies or antigen-binding fragments thereof can inhibit the interaction between αvβ5 and vitronectin; can inhibit the interaction between αvβ5 and LAP of TGF-β; can inhibit the activation of TGF-β; can bind rat, mouse, cynomolgus, and human αvβ5; can bind to αvβ5 recombinantly expressed on the cell surface (e.g., of BaF3) with a KD of 0.01 to 2 nM (e.g., 0.02, 0.04, 0.06, 0.08, 1.0, 1.2, 1.4, 1.6, 1.8 nM); bind to recombinant αvβ5 with an apparent affinity of about 5 pM to about 500 pM (e.g., 25 pM to 500 pM, 25 pM to 150 pM, 50 pM to 100 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 125 pM, or 150 pM); inhibit αvβ5 binding to vitronectin in an ELISA assay using purified proteins with an IC₅₀ value of about 1 nM to about 5 nM (e.g., 1 nM, 1.5 nM, 2.0 nM, 2.2 nM, 2.3 nM, 2.4 nM, 2.5 nM, 2.6 nM, 2.7 nM, 2.8 nM, 2.9 nM, 3.0 nM, 3.2 nM, 3.5 nM, 3.8 nM, 4.0 nM, 4.5 nM, or 5.0 nM); and/or can inhibit αvβ5 binding to vitronectin in a cell adhesion assay with an IC₅₀ of 0.1 to 2 nM (e.g., 0.2, 0.3, 0.4, 0.5, 0.6, 0.7, 0.8, 0.9, 1.0, 1.1, 1.2, 1.3, 1.4, 1.5, 1.6, 1.7, or 1.8 nM).

In certain embodiments, the anti-αvβ5 antibodies or antigen-binding fragments thereof comprise the VH and VL amino acid sequences set forth in SEQ ID NOs:5 and 10, respectively; or SEQ ID NOs:6 and 10, respectively; or SEQ ID NOs:5 and 8, respectively; or SEQ ID NOs:5 and 9, respectively; or SEQ ID NOs:3 and 10, respectively.

In some embodiments the VH and or VL region can be linked to a constant region (e.g., a wild-type human Fc region or an Fc region that includes one or more alterations). In some embodiments, the antibody has a light chain constant region derived from a human kappa sequence. In some embodiments, the antibody has a light chain constant region derived from a human lambda sequence. In a specific embodiment, the light chain constant region comprises a human subgroup kappa 1 sequence. In certain embodiments, the antibody has an isotype selected from the group consisting of IgG1, IgG2, IgG3, and IgG4. The heavy chain constant region can be a wild-type human Fc region, or a human Fc region that includes one or more amino acid substitutions. The antibodies can have mutations that stabilize the disulfide bond between the two heavy chains of an immunoglobulin, such as mutations in the hinge region of IgG4, as disclosed in the art (e.g., Angal et al., Mol. Immunol., 30:105-08 (1993)). See also, e.g., U.S. 2005-0037000. The heavy chain constant region can also have substitutions that modify the properties of the antibody (e.g., decrease one or more of: Fc receptor binding, antibody glycosylation, deamidation, binding to complement, or methionine oxidation). In some instances, the antibodies may have mutations such as those described in U.S. Pat. Nos. 5,624,821 and 5,648,260. In some embodiments, the antibody is modified to reduce or eliminate effector function. In some embodiments, the heavy chain constant region has one or more of the following mutations: S228P; N297Q; and T299A (numbering according to Kabat). The heavy chain constant region can be chimeric, e.g., the Fc region can comprise the CH1 and CH2 domains of an IgG antibody of the IgG4 isotype, and the CH3 domain from an IgG antibody of the IgG1isotype (see, e.g., U.S. Patent Appl. No. 2012/0100140A1 which is incorporated by reference in its entirety herein). In a specific embodiment, the humanized anti-αvβ5 antibodies described herein have a chimeric constant region comprising the CH1 and CH2 domains of an IgG antibody of the IgG4 isotype, and the CH3 domain from an IgG antibody of the IgG1isotype and further contain the S228P and N297Q mutations (numbering according to Kabat).

Non-limiting examples of humanized anti-αvβ5 antibodies of this disclosure include an antibody comprising the heavy chain amino acid sequence set forth in SEQ ID NO:69 and the light chain amino acid sequence set forth in SEQ ID NO:70; an antibody comprising the heavy chain amino acid sequence set forth in SEQ ID NO:69 and the light chain amino acid sequence set forth in SEQ ID NO:82; an antibody comprising the heavy chain amino acid sequence set forth in SEQ ID NO:80 and the light chain amino acid sequence set forth in SEQ ID NO:82; and an antibody comprising the heavy chain amino acid sequence set forth in SEQ ID NO:81 and the light chain amino acid sequence set forth in SEQ ID NO:70.

The antibodies or antigen binding fragments thereof described herein can be linked to a another agent (e.g., a fluorescent moiety, a radioactive molecule, a drug, a micro RNA, a cytotoxic agent). Cytotoxic agents can be e.g., a radionuclide, a biotoxin, an enzymatically active toxin, a cytostatic agent, a prodrug, an immunologically active ligand, a cytokines, an alkylating agent, an antimetabolilte, an anti-proliferative agent, a tubulin binding agent, a hormone, or a hormone antagonist. Exemplary cytotoxic agents include ⁹⁰Y, ¹³¹I, Monomethyl Auristatin E (MMAE), mertansine (DM1), DM4, diphtheria toxin, Pseudomonas exotoxin (PE38), and A chain of ricin. In a specific embodiment, the cytotoxic agent is a maytansinoid.

Antibodies can be selected for use based on improved potency, higher affinity or avidity for β5, and/or reduced immunogenicity than previously known αvβ5 antibodies. Methods of determining potency, affinity or avidity, and immunogenicity of antibodies are within the skill of the ordinary artisan.

### Methods of Obtaining Anti-αvβ5 Antibodies

Antibodies, such as those described above, can be made, for example, by preparing and expressing synthetic genes that encode the recited amino acid sequences. Methods of generating variants (e.g., comprising amino acid substitutions) of any of the anti-αvβ5 antibodies are well known in the art. These methods include, but are not limited to, preparation by site-directed (or oligonucleotide-mediated) mutagenesis, PCR mutagenesis, and cassette mutagenesis of a prepared DNA molecule encoding the antibody or any portion thereof (e.g., a framework region, a CDR (an alternate CDR), a constant region). Site-directed mutagenesis is well known in the art (see, e.g., Carter et al., Nucl. Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nucl. Acids Res. 17:723-733 (1989). Another method for preparing sequence variants, cassette mutagenesis, is based on the technique described by Wells et al., Gene, 34:315-323 (1985).

### Affinity Maturation

In one embodiment, an anti-αvβ5 antibody or antigen-binding fragment thereof described herein is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used.

In some implementations, the mutagenesis is targeted to regions known or likely to be at the binding interface. If, for example, the identified binding proteins are antibodies, then mutagenesis can be directed to the CDR regions (or alternate CDR regions) of the heavy or light chains as described herein. Further, mutagenesis can be directed to framework regions near or adjacent to the CDRs, e.g., framework regions, particularly within 10, 5, or 3 amino acids of a CDR (or alternate CDR) junction. In the case of antibodies, mutagenesis can also be limited to one or a few of the CDRs (or alternate CDRs), e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR (or alternate CDR) region. For example, the germline CDR (or alternate CDR) residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Grit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half life by providing a salvage receptor binding epitope.

In one embodiment, an antibody has CDR sequences (e.g., a Chothia or Kabat CDR) that differ from those of SEQ ID NOs:13-18. CDR sequences that differ from those of the humanized ALULA antibodies described herein include amino acid changes, such as substitutions of 1, 2, or 3 amino acids if a CDR is 5-7 amino acids in length, or substitutions of 1, 2, 3, 4, or 5 of amino acids in the sequence of a CDR if a CDR is 10 amino acids or greater in length. The amino acid that is substituted can have similar charge, hydrophobicity, or stereochemical characteristics. In some embodiments, the amino acid substitution(s) is a conservative substitution. In other embodiments, the amino acid substitution(s) is a non-conservative substitution. Such substitutions are within the ordinary skill of an artisan. The antibody or antibody fragments thereof that contain the substituted CDRs can be screened to identify antibodies having one or more of the features described herein (e.g., specifically binding to αvβ5, inhibiting the binding of αvβ5 to vitronectin).

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J. Immun., 147:2657-62 (1991); Morgan et al., Immunology, 86:319-24 (1995)), or changing the species from which the constant region is derived. In some instances, the antibodies or antigen binding fragments thereof have one to twenty-six, (i.e., 1, 2, 3, 4, 5, 6, 7, 8, 9, 10, 11, 12, 13, 14, 15, 16, 17, 18, 19, 20, 21, 22, 23, 24, 25, or 26) of the following amino acids: (a) in the variable heavy chain: valine at position 4, glutamine at position 5, glutamine at position 6, glutamic acid at position 16, lysine at position 23, lysine at position 38, lysine at position 66, alanine at position 67, leucine at position 69, alanine at position 71, valine at position 72, threonine at position 73, proline or serine at position 75, and/or alanine at position 78; and (b) in the variable light chain: asparagine at position 1, leucine at position 11, threonine at position 12, valine at position 13, methionine at position 21, serine at position 22, serine at position 43, aspartic acid at position 60, threonine at position 63, valine at position 78, alanine at position 100, and/or leucine at position 104 (the numbering is according to Kabat). In one embodiment, the antibodies or antigen binding fragments thereof comprising the CDRs of ALULA have a glutamic acid at position 16 of the variable heavy chain. In another embodiment, the antibodies or antigen binding fragments thereof described herein comprise the CDRs of ALULA and have a glutamic acid at position 16, valine at position 4, glutamine at position 6, lysine at position 23, lysine at position 66, alanine at position 71, threonine at position 73, proline at position 75, and alanine at position 78 of the variable heavy chain; and asparagine at position 1, leucine at position 11, methionine at position 21, serine at position 22, serine at position 43, aspartic acid at position 60, and threonine at position 63 of the variable light chain.

The anti-αvβ5 antibodies can be in the form of full length antibodies, or in the form of low molecular weight forms (e.g., biologically active antibody fragments or minibodies) of the anti-αvβ5 antibodies, e.g., Fab, Fab′, F(ab′)2, Fv, Fd, dAb, scFv, and sc(Fv)2. Other anti-αvβ5 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-αvβ5 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-αvβ5 antibody or antigen-binding fragment thereof comprising at least one deamidation site, wherein the pH of the composition is from about 5.0 to about 6.5, such that, e.g., at least about 90% of the anti-αvβ5 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-αvβ5 antibody or antigen-binding fragment thereof, means the presence of both the desired anti-αvβ5 antibody or antigen-binding fragment thereof and one or more acidic variants thereof. The acidic variants may comprise predominantly deamidated anti-αvβ5 antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

In certain embodiments, an anti-αvβ5 antibody or antigen-binding fragment thereof or low molecular weight antibodies thereof specifically binds to β5, inhibits the binding of αvβ5 to vitronectin, inhibits the binding of αvβ5 to LAP of TGF-3, inhibits the activation of TGF-ρ, inhibits TGF-β signaling, and/or reduces the severity of symptoms when administered to human patients having one or more of: acute kidney injury, acute lung injury, stroke (cerebral hemorrhage), acute respiratory distress syndrome, pulmonary edema, lung fibrosis (e.g., idiopathic pulmonary fibrosis (IPF), usual interstitial pneumonia (UIP)), sepsis, myocardial infarction, cancer (e.g., pancreatic cancer, lung cancer, breast cancer, colorectal cancer, head and neck cancer, esophageal cancer, skin cancer, prostate cancer, cervical cancer, colon cancer, ovarian cancer, and endometrial cancer), and ocular neovascularization disease. In some embodiments, the antibodies or the antigen-binding fragments thereof described herein inhibit or reduce angiogenesis and thereby prevent or retard the development of cancer. In one embodiment, the anti-αvβ5 antibody or antigen-binding fragment thereof or low molecular weight antibodies thereof inhibit disease development in an idiopathic pulmonary fibrosis model (Degryse et al., Am J Med Sci., 0.341(6):444-9 (2011)). In another embodiment, the antibodies or the antigen-binding fragments thereof described herein inhibit vascular permeability in models of injury-induced vascular permeability (e.g., VEGF, ventilator-induced, IL-1β, ischemia-reperfusion, LPS, cecal-ligation with puncture).

### Antibody Fragments

Antibody fragments (e.g., Fab, Fab′, F(ab′)2, Facb, and Fv) of αvβ5-binding antibodies may be prepared by proteolytic digestion of intact αvβ5 antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Minibodies of anti-αvβ5antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161).

Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A, 85:5879-5883 (1988); and Pluckthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-αvβ5 antibody or antigen-binding fragment thereof described herein.

An sc(Fv)2 is a minibody in which two VHs and two VLs are linked by a linker to form a single chain (Hudson, et al., J. Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order. Examples of arrangements are listed below:

[VL] linker [VH] linker [VH] linker [VL]

[VH] linker [VL] linker [VL] linker [VH]

[VH] linker [VH] linker [VL] linker [VL]

[VL] linker [VL] linker [VH] linker [VH]

[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18) can be used as a linker. Examples of such peptide linkers include: Ser; Gly Ser; Gly Gly Ser; Ser Gly Gly; Gly Gly Gly Ser (SEQ ID NO:68); Ser Gly Gly Gly (SEQ ID NO:73); Gly Gly Gly Gly Ser (SEQ ID NO:74); Ser Gly Gly Gly Gly (SEQ ID NO:75); Gly Gly Gly Gly Gly Ser (SEQ ID NO:76); Ser Gly Gly Gly Gly Gly (SEQ ID NO:77); Gly Gly Gly Gly Gly Gly Ser (SEQ ID NO:78); Ser Gly Gly Gly Gly Gly Gly (SEQ ID NO:79); (Gly Gly Gly Gly Ser (SEQ ID NO:74)ₙ, wherein n is an integer of one or more; and (Ser Gly Gly Gly Gly (SEQ ID NO:75)ₙ, wherein n is an integer of one or more.

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the minibodies may include modifications such as substitutions, deletions, additions, and/or insertions. For example, the modification may be in one or more of the CDRs (or alternate CDRs) of the anti-αvβ5 antibody or antigen-binding fragment thereof. In certain embodiments, the modification involves one, two, or three amino acid substitutions in one or more CDRs (or alternate CDRs) and/or framework regions of the VH and/or VL domain of the anti-αvβ5 minibody. Such substitutions are made to improve the binding, functional activity, and/or reduce immunogenicity of the anti-αvβ5 minibody. In certain embodiments, the substitutions are conservative amino acid substitutions. In other embodiments, one, two, or three amino acids of the CDRs (or alternate CDRs) of the anti-αvβ5 antibody or antigen-binding fragment thereof may be deleted or added as long as there is αvβ5 binding and/or functional activity when VH and VL are associated. The modified minibodies can inhibit αvβ5 binding to vitronectin; inhibit αvβ5 binding to LAP of TGF-β; and/or inhibit TGF-β signaling.

### Bispecific Antibodies

Bispecific antibodies are antibodies that have binding specificities for at least two different epitopes. Exemplary bispecific antibodies may bind to two different epitopes of the αvβ5 protein. Other such antibodies may combine a αvβ5 binding site with a binding site for another protein (e.g., αvβ6, αvβ8, tumor specific antigens (e.g., alphafetoprotein (AFP), carcinoembryonic antigen (CEA), CA-125, MUC-1, Epithelial tumor antigen (ETA), tyrosinase, Melanoma-associated antigen (MAGE)-1, MAGE-3, BAGE-1, GAGE-1, GnTV, KM-HN-1, KK-LC-1, LAGE-1, NA88-A, NY-ESO-1, SAGE, Sp17, SSX-2, TAG-1, TRAG-3, TRP2, XAGE-1b, HPV 16, HPV E6, HPV E7, TAG-72, L6-antigen, CD19, CD22, CD37, CD52, EGF receptor, HER 2 receptor, Lewis Y), T-cell antigens (e.g., CD2, CD3, CD5, CD6, CD7, TCR)). Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, fluorescent substances, luminescent substances, haptens, enzymes, metal chelates, and drugs.

In one embodiment, to improve the cytotoxic actions of anti-αvβ5 antibodies (and antigen-binding fragments thereof) and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (e.g., use of antibody fragments, murine sequences, and/or IgG3 or IgG4 isotypes). Exemplary radioisotopes include: ⁹⁰Y, ¹²⁵I, ¹³¹I, ¹²³I, ¹¹¹I, ¹⁰⁵Rh, ¹⁵³Sm, ⁶⁷Cu, ⁶⁷Ga, ¹⁶⁶Ho, ¹⁷⁷Lu, ¹⁸⁶Re, and ¹⁸⁸Re. Cytotoxic agents that can be used include cytotoxic drugs which are used for cancer therapy. As used herein, “a cytotoxic agent” means any agent that is detrimental to the growth and proliferation of cells and may act to reduce, inhibit, or destroy a cell or malignancy. Exemplary cytotoxic agents include, but are not limited to, radionuclides, biotoxins, enzymatically active toxins, cytostatic or cytotoxic therapeutic agents (e.g., alkylating agents, antimetabolites, anti-proliferative agents, tubulin binding agents, hormones and hormone antagonists), prodrugs, immunologically active ligands and biological response modifiers such as cytokines. Any cytotoxin that acts to retard or slow the growth of immunoreactive cells or malignant cells is within the scope of the present invention. Exemplary cytostatics include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. In one embodiment, the cytotoxic agent that is conjugated to an antibody or antigen-binding fragment described herein is a maytansinoid. Maytansinoids are known in the art to include maytansine, maytansinol, C-3 esters of maytansinol, and other maytansinol analogues and derivatives (see, e.g., U.S. Pat. Nos. 5,208,020 and 6,441,163). C-3 esters of maytansinol can be naturally occurring or synthetically derived. Moreover, both naturally occurring and synthetic C-3 maytansinol esters can be classified as a C-3 ester with simple carboxylic acids, or a C-3 ester with derivatives of N-methyl-L-alanine, the latter being more cytotoxic than the former. Synthetic maytansinoid analogues also are known in the art and described in, for example, Kupchan et al., J. Med. Chem., 21, 31-37 (1978). Methods for generating maytansinol and analogues and derivatives thereof are described in, for example, U.S. Pat. No. 4,151,042. In certain embodiments, the maytansinoids comprise a linking moiety that contains a reactive chemical group (e.g., C-3 esters of maytansinol and its analogs where the linking moiety contains a disulfide bond and the attachment moiety comprises a N-succinimidyl or N-sulfosuccinimidyl ester). In certain embodiments, the maytansinoid conjugated with the antibodies or antigen-binding described herein is N²′-deacetyl-N²′-(-3-mercapto-1-oxopropyl)-maytansine (DM1) or N²′-deacetyl-N²′-(4-mercapto-4-methyl-1-oxopentyl)-maytansine (DM4). In certain other embodiments, cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of these families include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. In other embodiments, the cytotoxic agents include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide can also be conjugated with the antibodies or antigen-binding fragments thereof described herein. In certain embodiments, the cytotoxic agent comprises a member or derivative of the enediyne family of anti-tumor antibiotics, including calicheamicin, esperamicins, or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins, and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs. In further embodiments, the antibodies or antigen-binding fragments thereof can also be associated with a biotoxin such as ricin subunit A, abrin, diptheria toxin, botulinum, cyanginosins, saxitoxin, shigatoxin, tetanus, tetrodotoxin, trichothecene, verrucologen, or a toxic enzyme. Such biotoxins can be made using genetic engineering techniques that allow for direct expression of the antibody-toxin construct. One skilled in the art could readily form such constructs using conventional techniques. Methods of conjugating cytotoxic agents are well known in the art (see, e.g., U.S. Pat. No. 8,021,661).

In certain embodiments, an anti-αvβ5 antibody or antigen-binding fragment thereof are modified with a moiety that improves its stabilization and/or retention in circulation, e.g., in blood, serum, or other tissues, e.g., by at least 1.5, 2, 5, 10, or 50 fold. For example, the anti-αvβ5antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-αvβ5 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) (see, e.g., Chapman et al., Nature Biotechnology, 17: 780-783 (1999), or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides. The efficacy of a therapeutic antibody can be improved by increasing its serum persistence, thereby allowing higher circulating levels, less frequent administration, and reduced doses. The half-life of an IgG depends on its pH-dependent binding to the neonatal receptor FcRn. FcRn, which is expressed on the surface of endothelial cells, binds the IgG in a pH-dependent manner and protects it from degradation. Some antibodies that selectively bind the FcRn at pH 6.0, but not pH 7.4, exhibit a higher half-life in a variety of animal models. In certain embodiments, the antibodies of the present disclosure have one or more mutations at the interface between the CH2 and CH3 domains, such as T250Q/M428L and M252Y/S254T/T256E+H433K/N434F (the numbering is according to the EU index), which increase the binding affinity to FcRn and the half-life of IgG1 in vivo. In other embodiments, the antibodies herein have a modified Fc region comprising at least one modification relative to a wild-type human Fc region, where the modification is selected from the group consisting of 434S, 252Y/428L, 252Y/434S, and 428L/434S, and the numbering is according to the EU index.

The antibodies or antigen-binding fragments thereof can also be conjugated to siRNAs, miRNAs, or anti-miRs to deliver the siRNA, miRNA, or anti-miR to cells expressing αvβ5 (see, e.g., Song et al., Nat. Biotechnol., 23(6):709-17 (2005); Schneider et al., Molecular Therapy Nucleic Acids, 1:e46 (2012)). The siRNAs, miRNAs, or anti-miRs can target TGF-β or components of the TGF-β signaling pathway. In some embodiments, the siRNAs, miRNAs, or anti-miRs can target genes involved in the disease being treated (e.g., lung fibrosis, acute lung injury, cancer). For example, to treat lung fibrosis, one or more of the following can be targeted to αvβ5-expressing cells using αvβ5 antibodies or antigen-binding fragments thereof conjugated to: anti-miRs to microRNAs such as: miR-142-3p, miR-155, miR-192, miR-199a/b, miR-208, miR-21, miR-215, miR-216, miR-217, miR-23a, miR-27a, miR-27b, miR-32, miR-338, miR-34a, miR-377, miR-382; or conjugated to microRNAs such as: let-7d, miR-107, miR-132, miR-133, miR-141, miR-15b, miR-16, miR-150, miR-18a, miR-19a/b, miR-194, miR-200a/b, miR-204, miR-211, miR-26a/b, miR-29a/b/c, miR-30c, miR-335, miR-449a/b, and miR-590. To treat acute lung injury, one or more of the following can be targeted to αvβ5-expressing cells using αvβ5 antibodies or antigen-binding fragments thereof conjugated to: miR-127, miR-16, and/or miR-199a. In a specific embodiment, anti-miR-21 conjugated to humanized αvβ5 antibodies or antigen-binding fragments thereof can be used to treat cancer (e.g., hepatocellular carcinoma); and humanized αvβ5 antibodies or antigen-binding fragments thereof conjugated to anti-miR-10b can be used to treat cancers such as glioblastoma.

The above-described conjugated antibodies can be prepared by performing chemical modifications on the antibodies or the lower molecular weight forms thereof described herein. Methods for modifying antibodies are well known in the art (e.g., U.S. Pat. No. 5,057,313 and U.S. Pat. No. 5,156,840).

### Methods of Producing Antibodies

The αvβ5 antibodies or antibody binding fragments thereof of this disclosure may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS, 3T3). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, M13-series vectors, pUC-series vectors, pBR322, pBluescript, pCR-Script, pGEX-5×-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, 293, 293T, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, the antibodies or antigen-binding fragments thereof are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-αvβ5 antibody, or two separate recombinant expression vectors each separately encoding the antibody heavy chain and the antibody light chain of an anti-αvβ5 antibody, is introduced into dhfr⁻ CHO cells by calcium phosphate-mediated transfection. In one embodiment, the CHO cell is CHO DG44i. In another embodiment, the CHO cell is CHO K1 GS. Within the recombinant expression vector(s), the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector(s) also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Characterization of the Antibodies

The αvβ5-binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-αvβ5 antibody) and a target (e.g., β5) can be analyzed using the OCTET® systems. In this method, one of several variations of instruments (e.g., OCTET® QKe and QK), made by the FortéBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-αvβ5 antibody) and a target (e.g., β5) can be analyzed using Surface Plasmon Resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to human αvβ5 or β5 using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

Anti-αvβ5Antibodies with Modified Effector Function

The interaction of antibodies and antibody-antigen complexes with cells of the immune system triggers a variety of responses, referred to herein as effector functions. Immune-mediated effector functions include two major mechanisms: antibody-dependent cell-mediated cytotoxicity (ADCC) and complement-dependent cytotoxicity (CDC). Both of them are mediated by the constant region of the immunoglobulin protein. The antibody Fc domain is, therefore, the portion that defines interactions with immune effector mechanisms.

IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. In some clinical applications these responses are crucial for the efficacy of a monoclonal antibody. In others they provoke unwanted side effects such as inflammation and the elimination of antigen-bearing cells. Accordingly, the present invention further relates to αvβ5-binding proteins, including antibodies, with altered, e.g., increased or reduced effector functions.

Effector function of an anti-αvβ5 antibody of the present invention may be determined using one of many known assays. The anti-αvβ5 antibody's effector function may be increased or reduced relative to a second anti-αvβ5 antibody. In some embodiments, the second anti-αvβ5 antibody may be any antibody that binds αvβ5 specifically. In certain embodiments, the second anti-αvβ5 antibody may be any humanized antibody that specifically binds αvβ5. In other embodiments, the second αvβ5-specific antibody may be any of the antibodies of the invention, such as the antibodies described in Examples 2 and 8. In other embodiments, where the anti-αvβ5 antibody of interest has been modified to reduce effector function, the second anti-αvβ5 antibody may be the unmodified or parental version of the antibody.

Effector functions include ADCC, whereby antibodies bind Fc receptors on cytotoxic T cells, natural killer (NK) cells, or macrophages leading to cell death, and CDC, which is cell death induced via activation of the complement cascade (reviewed in Daeron, Annu. Rev. Immunol., 15:203-234 (1997); Ward and Ghetie, Therapeutic Immunol., 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195).

Effector functions can be avoided by using antibody fragments lacking the Fc domain such as Fab, Fab′2, or single chain Fv. An alternative is to use the IgG4 subtype antibody, which binds to FcγRI but which binds poorly to C1q and FcγRII and RIII. However, IgG4 antibodies may form aggregates since they have poor stability at low pH compared with IgG1 antibodies. The stability of an IgG4 antibody can be improved by substituting arginine at position 409 (according to the EU index proposed by Kabat et al., Sequences of proteins of immunological interest, 1991, 5^{th}) with any one of: lysine, methionine, threonine, leucine, valine, glutamic acid, asparagine, phenylalanine, tryptophan, or tyrosine. Alternatively, and or in addition, the stability of an IgG4 antibody can be improved by substituting a CH3 domain of an IgG4 antibody with a CH3 domain of an IgG1 antibody, or by substituting the CH2 and CH3 domains of IgG4 with the CH2 and CH3 domains of IgG1. Accordingly, the anti-αvβ5 antibodies of the present invention that are of IgG4 isotype can include modifications at position 409 and/or replacement of the CH2 and/or CH3 domains with the IgG1 domains so as to increase stability of the antibody while decreasing effector function. The IgG2 subtype also has reduced binding to Fc receptors, but retains significant binding to the H131 allotype of FcγRIIa and to C1q. Thus, additional changes in the Fc sequence may be required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated (i.e., increased or decreased) by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FcεR, for IgA as FcαR and so on. Three subclasses of FcγR have been identified: FcγRI (CD64), FcγRII (CD32) and FcγRIII (CD16). Both FcγRII and FcγRIII have two types: FcγRIIa (CD32a) and FcγRIIB (CD32b); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms IIa, IIb1, IIb2 IIb3, and IIc.

The binding site on human and murine antibodies for FcγR has been previously mapped to the so-called “lower hinge region” consisting of residues G233-S239 (EU index numbering as in Kabat et al., Sequences of Proteins of Immunological Interest, 5th Ed. Public Health Service, National Institutes of Health, Bethesda, Md. (1991), Woof et al., Molec. Immunol. 23:319-330 (1986); Duncan et al., Nature 332:563 (1988); Canfield and Morrison, J Exp. Med. 173:1483-1491 (1991); Chappel et al., Proc. Natl. Acad. Sci USA 88:9036-9040 (1991)). Of residues G233-5239, P238 and S239 are among those cited as possibly being involved in binding. Other residues involved in binding to FcγR are: G316-K338 (Woof et al., Mol. Immunol., 23:319-330 (1986)); K274-R301 (Sarmay et al., Molec. Immunol. 21:43-51 (1984)); Y407-R416 (Gergely et al., Biochem. Soc. Trans. 12:739-743 (1984) and Shields et al., J Biol Chem 276: 6591-6604 (2001), Lazar G A et al., Proc Natl Acad Sci 103: 4005-4010 (2006)); N297; T299; E318; L234-5239; N265-E269; N297-T299; and A327-I332. These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-αvβ5 antibodies of the present invention include modifications of one or more of the aforementioned residues to increase or decrease effector function as needed.

Another approach for altering monoclonal antibody effector function include mutating amino acids on the surface of the monoclonal antibody that are involved in effector binding interactions (Lund, J., et al. (1991) J. Immunol. 147(8): 2657-62; Shields, R. L. et al. (2001) J. Biol. Chem. 276(9): 6591-604).

Methods of increasing effector function of antibodies are well known in the art (see, e.g., Kelley et al., Methods Mol. Biol., 901:277-93 (2012); Natsume et al., Drug Des Devel Ther., 3:7-16 (2009); U.S. Pat. No. 8,188,231, U.S. Pat. No. 7,960,512). In one embodiment, the αvβ5 antibodies have one, two, three, four, five, six, seven, or more amino acid substitutions at a position selected from the group consisting of 221, 222, 223, 224, 225, 227, 228, 230, 231, 232, 233, 234, 235, 236, 237, 238, 239, 240, 241, 243, 244, 245, 246, 247, 249, 255, 258, 260, 262, 263, 264, 265, 266, 267, 268, 269, 270, 271, 272, 273, 274, 275, 276, 278, 280, 281, 282, 283, 284, 285, 286, 288, 290, 291, 292, 293, 294, 295, 296, 297, 298, 299, 300, 301, 302, 303, 304, 305, 313, 317, 318, 320, 322, 323, 324, 325, 326, 327, 328, 329, 330, 331, 332, 333, 334, 335, 336, and 337, wherein the numbering of the residues in the Fc region is that of the EU index as in Kabat. In certain embodiments, the αvβ5 antibodies have one, two, three, four, five, six, seven, or more of the amino acid substitutions selected from the group consisting of: D221K, D221Y, K222E, K222Y, T223E, T223K, H224E, H224Y, T225E, T225K, T225W, P227E, P227G, P227K, P227Y, P228E, P228G, P228K, P228Y, P230A, P230E, P230G, P230Y, A231E, A231G, A231K, A231P, A231Y, P232E, P232G, P232K, P232Y, E233A, E233D, E233F, E233G, E233H, E233I, E233K, E233L, E233M, E233N, E233Q, E233R, E233S, E233T, E233V, E233W, E233Y, L234A, L234D, L234E, L234F, L234G, L234H, L234I, L234K, L234M, L234N, L234P, L234Q, L234R, L234S, L234T, L234V, L234W, L234Y, L235A, L235D, L235E, L235F, L235G, L235H, L235I, L235K, L235M, L235N, L235P, L235Q, L235R, L235S, L235T, L235V, L235W, L235Y, G236A, G236D, G236E, G236F, G236H, G236I, G236K, G236L, G236M, G236N, G236P, G236Q, G236R, G236S, G236T, G236V, G236W, G236Y, G237D, G237E, G237F, G237H, G237I, G237K, G237L, G237M, G237N, G237P, G237Q, G237R, G237S, G237T, G237V, G237W, G237Y, P238D, P238E, P238F, P238G, P238H, P238I, P238K, P238L, P238M, P238N, P238Q, P238R, P238S, P238T, P238V, P238W, P238Y, S239D, S239E, S239F, S239G, S239H, S239I, S239K, S239L, S239M, S239N, S239P, S239Q, S239R, S239T, S239V, S239W, S239Y, V240A, V240I, V240M, V240T, F241D, F241E, F241L, F241R, F241S, F241W, F241Y, F243E, F243H, F243L, F243Q, F243R, F243W, F243Y, P244H, P245A, K246D, K246E, K246H, K246Y, P247G, P247V, D249H, D249Q, D249Y, R255E, R255Y, E258H, E258S, E258Y, T260D, T260E, T260H, T260Y, V262A, V262E, V262F, V262I, V262T, V263A, V263I, V263M, V263T, V264A, V264D, V264E, V264F, V264G, V264H, V264I, V264K, V264L, V264M, V264N, V264P, V264Q, V264R, V264S, V264T, V264W, V264Y, D265F, D265G, D265H, D265I, D265K, D265L, D265M, D265N, D265P, D265Q, D265R, D265S, D265T, D265V, D265W, D265Y, V266A, V266I, V266M, V266T, S267D, S267E, S267F, S267H, S267I, S267K, S267L, S267M, S267N, S267P, S267Q, S267R, S267T, S267V, S267W, S267Y, H268D, H268E, H268F, H268G, H268I, H268K, H268L, H268M, H268P, H268Q, H268R, H268T, H268V, H268W, E269F, E269G, E269H, E269I, E269K, E269L, E269M, E269N, E269P, E269R, E269S, E269T, E269V, E269W, E269Y, D270F, D270G, D270H, D270I, D270L, D270M, D270P, D270Q, D270R, D270S, D270T, D270W, D270Y, P271A, P271D, P271E, P271F, P271G, P271H, P271I, P271K, P271L, P271M, P271N, P271Q, P271R, P271S, P271T, P271V, P271W, P271Y, E272D, E272F, E272G, E272H, E272I, E272K, E272L, E272M, E272P, E272R, E272S, E272T, E272V, E272W, E272Y, V273I, K274D, K274E, K274F, K274G, K274H, K274I, K274L, K274M, K274N, K274P, K274R, K274T, K274V, K274W, K274Y, F275L, F275W, N276D, N276E, N276F, N276G, N276H, N276I, N276L, N276M, N276P, N276R, N276S, N276T, N276V, N276W, N276Y, Y278D, Y278E, Y278G, Y278H, Y278I, Y278K, Y278L, Y278M, Y278N, Y278P, Y278Q, Y278R, Y278S, Y278T, Y278V, Y278W, D280G, D280K, D280L, D280P, D280W, G281D, G281E, G281K, G281N, G281P, G281Q, G281Y, V282E, V282G, V282K, V282P, V282Y, E283G, E283H, E283K, E283L, E283P, E283R, E283Y, V284D, V284E, V284L, V284N, V284Q, V284T, V284Y, H285D, H285E, H285K, H285Q, H285W, H285Y, N286E, N286G, N286P, N286Y, K288D, K288E, K288Y, K290D, K290H, K290L, K290N, K290W, P291D, P291E, P291G, P291H, P291I, P291Q, P291T, R292D, R292E, R292T, R292Y, E293F, E293G, E293H, E293I, E293L, E293M, E293N, E293P, E293R, E293S, E293T, E293V, E293W, E293Y, E294F, E294G, E294H, E294I, E294K, E294L, E294M, E294P, E294R, E294S, E294T, E294V, E294W, E294Y, Q295D, Q295E, Q295F, Q295G, Q295H, Q295I, Q295M, Q295N, Q295P, Q295R, Q295S, Q295T, Q295V, Q295W, Q295Y, Y296A, Y296D, Y296E, Y296G, Y296H, Y296I, Y296K, Y296L, Y296M, Y296N, Y296Q, Y296R, Y296S, Y296T, Y296V, N297D, N297E, N297F, N297G, N297H, N297I, N297K, N297L, N297M, N297P, N297Q, N297R, N297S, N297T, N297V, N297W, N297Y, S298D, S298E, S298F, S298H, S298I, S298K, S298M, S298N, S298Q, S298R, S298T, S298W, S298Y, T299A, T299D, T299E, T299F, T299G, T299H, T299I, T299K, T299L, T299M, T299N, T299P, T299Q, T299R, T299S, T299V, T299W, T299Y, Y300A, Y300D, Y300E, Y300G, Y300H, Y300K, Y300M, Y300N, Y300P, Y300Q, Y300R, Y300S, Y300T, Y300V, Y300W, R301D, R301E, R301H, R301Y, V302I, V303D, V303E, V303Y, S304D, S304H, S304L, S304N, S304T, V305E, V305T, V305Y, W313F, K317E, K317Q, E318H, E318L, E318Q, E318R, E318Y, K320D, K320F, K320G, K320H, K320I, K320L, K320N, K320P, K320S, K320T, K320V, K320W, K320Y, K322D, K322F, K322G, K322H, K322I, K322P, K322S, K322T, K322V, K322W, K322Y, V323I, S324D, S324F, S324G, S324H, S324I, S324L, S324M, S324P, S324R, S324T, S324V, S324W, S324Y, N325A, N325D, N325E, N325F, N325G, N325H, N325I, N325K, N325L, N325M, N325P, N325Q, N325R, N325S, N325T, N325V, N325W, N325Y, K326I, K326L, K326P, K326T, A327D, A327E, A327F, A327H, A327I, A327K, A327L, A327M, A327N, A327P, A327R, A327S, A327T, A327V, A327W, A327Y, L328A, L328D, L328E, L328F, L328G, L328H, L328I, L328K, L328M, L328N, L328P, L328Q, L328R, L328S, L328T, L328V, L328W, L328Y, P329D, P329E, P329F, P329G, P329H, P329I, P329K, P329L, P329M, P329N, P329Q, P329R, P329S, P329T, P329V, P329W, P329Y, A330E, A330F, A330G, A330H, A330I, A330L, A330M, A330N, A330P, A330R, A330S, A330T, A330V, A330W, A330Y, P331D, P331F, P331H, P331I, P331L, P331M, P331Q, P331R, P331T, P331V, P331W, P331Y, I332A, I332D, I332E, I332F, I332H, I332K, I332L, I332M, I332N, I332P, I332Q, I332R, I332S, I332T, I332V, I332W, I332Y, E333F, E333H, E333I, E333L, E333M, E333P, E333T, E333Y, K334F, K334I, K334L, K334P, K334T, T335D, T335F, T335G, T335H, T335I, T335L, T335M, T335N, T335P, T335R, T335S, T335V, T335W, T335Y, 1336E, I336K, I336Y, S337E, S337H, and S337N, wherein the numbering of the residues in the Fc region is that of the EU index as in Kabat. In a particular embodiment, the αvβ5 antibodies comprise one, two, or three of the following mutations: S239D, S239D/I332E, S239D/I332E/A330L, S239D/I332E/G236A, S298A, A330L I332E, E333A, and K334A.

The presence of oligosaccharides—specifically, the N-linked oligosaccharide at asparigine-297 in the CH2 domain of IgG1—is important for binding to FcγR as well as C1q. Reducing the fucose content of antibodies improves effector function (see, e.g., U.S. Pat. No. 8,163,551). In certain embodiments the αvβ5 antibodies have reduced fucosylation and amino acid substitutions that increase effector function (e.g., one, two, or three of the following mutations: S298A; E333A, and K334A). Effector function can also be achieved by preparing and expressing the anti-αvβ5 antibodies described herein in the presence of alpha-mannosidase I inhibitors (e.g., kifunensine) at a concentration of the inhibitor of about 60-200 ng/mL (e.g., 60 ng/mL, 75 ng/mL, 100 ng/mL, 150 ng/ml). Antibodies expressed in the presence of alpha-mannosidase I inhibitors contain mainly oligomannose-type glycans and generally demonstrate increased ADCC activity and affinity for FcγRIIIA, but reduced C1q binding.

Anti-αvβ5 antibodies of the present disclosure with increased effector function include antibodies with increased binding affinity for one or more Fc receptors (FcRs) relative to a parent or non-variant anti-αvβ5 antibody. Accordingly, anti-αvβ5 antibodies with increased FcR binding affinity includes anti-αvβ5 antibodies that exhibit a 1.5-fold, 2-fold, 2.5-fold, 3-fold, 4-fold, or 5-fold or higher increase in binding affinity to one or more Fc receptors compared to a parent or non-variant anti-αvβ5 antibody. In some embodiments, an anti-αvβ5 antibody with increased effector function binds to an FcR with about 10-fold greater affinity relative to a parent or non-variant antibody. In other embodiments, an anti-αvβ5 antibody with increased effector function binds to an FcR with about 15-fold greater affinity or with about 20-fold greater affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-αvβ5 antibody with increased effector function exhibits a 1.5-fold, 2-fold, 2.5-fold, 3-fold, 4-fold, or 5-fold or higher increase in binding affinity to FcγRIIa.

In some instances, there is a need to eliminate effector function/complement activation in humanized ALULA, as (1) the presumed mechanism of action of ALULA is to inhibit ligand binding to αvβ5; (2) αvβ5 is widely expressed on most cell types; and (3) complement activation is well documented in the relevant clinical indications and may have a causative role in disease. Accordingly, the present invention further relates to αvβ5-binding antibodies with reduced effector functions. To reduce effector function, one can use combinations of different subtype sequence segments (e.g., IgG2 and IgG4 combinations) to give a greater reduction in binding to Fcγ receptors than either subtype alone (Armour et al., Eur. J. Immunol., 29:2613-1624 (1999); Mol. Immunol., 40:585-593 (2003)). A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260.

Anti-αvβ5 antibodies of the present invention with reduced effector function include antibodies with reduced binding affinity for one or more Fc receptors (FcRs) relative to a parent or non-variant anti-αvβ5 antibody. Accordingly, anti-αvβ5 antibodies with reduced FcR binding affinity includes anti-αvβ5 antibodies that exhibit a 1.5-fold, 2-fold, 2.5-fold, 3-fold, 4-fold, 5-fold, 10-fold, 20-fold, or 25-fold or higher decrease in binding affinity to one or more Fc receptors compared to a parent or non-variant anti-αvβ5 antibody. In some embodiments, an anti-αvβ5 antibody with reduced effector function binds to an FcR with about 10-fold less affinity relative to a parent or non-variant antibody. In other embodiments, an anti-αvβ5 antibody with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-αvβ5 antibody with reduced effector function exhibits a 1.5-fold, 2-fold, 2.5-fold, 3-fold, 4-fold, or 5-fold or higher decrease in binding affinity to FcγRIIa.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus, the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J. Immunol. Methods, 202:163 (1996), may be performed.

Various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. No. 5,648,260, and U.S. Pat. No. 5,624,821).

Anti-αvβ5 antibodies with improved C1q binding can comprise an amino acid substitution at one, two, three, or four of amino acid positions 326, 327, 333 and 334 of the human IgG Fc region, where the numbering of the residues in the IgG Fc region is that of the EU index as in Kabat. In one embodiment, the anti-αvβ5 antibodies include the following amino acid substitutions: K326W/E333S, which are known to increase binding of an IgG1 antibody to C1q (Steurer W. et al., J Immunol., 155(3):1165-74 (1995)).

Anti-αvβ5 antibodies with reduced C1q binding can comprise an amino acid substitution at one, two, three, or four of amino acid positions 270, 322, 329 and 331 of the human IgG Fc region, where the numbering of the residues in the IgG Fc region is that of the EU index as in Kabat. As an example in IgG1, two mutations in the COOH terminal region of the CH2 domain of human IgG1—K322A and P329A—do not activate the CDC pathway and were shown to result in more than a 100 fold decrease in C1q binding (U.S. Pat. No. 6,242,195).

Accordingly, in certain embodiments, an anti-αvβ5 antibody of the present invention exhibits reduced or increased binding to a complement protein relative to a second anti-αvβ5 antibody. In certain embodiments, an anti-αvβ5 antibody of the invention exhibits increased or reduced binding to C1q by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to a second anti-αvβ5 antibody.

Thus, in certain embodiments of the invention, one or more of these residues may be modified, substituted, or removed or one or more amino acid residues may be inserted so as to increase or decrease CDC activity of the anti-αvβ5 antibodies provided herein.

In certain other embodiments, the present invention provides an anti-αvβ5 antibody that exhibits reduced binding to one or more FcR receptors but that maintains its ability to bind complement (e.g., to a similar or, in some embodiments, to a lesser extent than a native, non-variant, or parent anti-αvβ5 antibody). Accordingly, an anti-αvβ5 antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind C1q and activate the complement cascade to at least some degree will reduce the risk of thromboembolic events while maintaining perhaps desirable effector functions. In alternative embodiments, an anti-αvβ5 antibody of the present invention exhibits reduced binding to one or more FcRs but maintains its ability to bind one or more other FcRs. See, for example, US 2007-0009523, 2006-0194290, 2005-0233382, 2004-0228856, and 2004-0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an anti-αvβ5 antibody may be modulated by altering properties of the constant region, and the Fc region in particular. In certain embodiments, the anti-αvβ5 antibody having decreased effector function is compared with a second antibody with effector function and which may be a non-variant, native, or parent antibody comprising a native constant or Fc region that mediates effector function.

A native constant region comprises an amino acid sequence identical to the amino acid sequence of a constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. The variant constant region can have decreased effector function.

Antibodies with altered (i.e., increased or decreased) effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns, may be achieved by manipulating the host cell and cell culture and expression conditions by which the antibody is produced.

Certain embodiments of the present invention relate to an anti-αvβ5 antibody comprising or consisting of one or more (1, 2, or 3) heavy chain CDR sequences (Kabat or alternate CDR) from any one of SEQ ID NOs:1-7. In one embodiment, the anti-αvβ5 antibody heavy chain CDR sequences comprise or consist of the amino acid sequences in SEQ ID NO:13, SEQ ID NO:14, and SEQ ID NO:15. These antibodies may also comprise or consist of one or more (1, 2, or 3) light chain CDR sequences (Kabat or alternate CDR) from any one of SEQ ID NOs:8-12. For example, the anti-αvβ5antibody light chain CDR sequences may comprise or consist of the amino acid sequences in SEQ ID NO:16, SEQ ID NO:17, and SEQ ID NO:18. The antibodies described herein may further comprise an Fc region that confers increased or reduced effector function compared to a native or parental Fc region. In certain embodiments, the Fc region of these antibodies is chimeric comprising the CH1 and CH2 domains of IgG4 and the CH3 domain of IgG1. These anti-αvβ5 antibodies (i) inhibit the interaction between αvβ5 and vitronectin; and/or (ii) inhibit the interaction between αvβ5 and LAP of TGF-β; and/or (iii) inhibit the activation of TGF-β; and/or (iv) bind with high affinity to human αvβ5.

In other embodiments, the disclosure provides an anti-αvβ5 antibody comprising a VL sequence comprising SEQ ID NO:10 and a VH sequence comprising SEQ ID NO:5, the antibody further comprising an Fc region or a variant Fc region that confers increased or reduced effector function compared to a native or parental Fc region. In a specific embodiment, the disclosure provides an anti-αvβ5 antibody comprising a light chain sequence comprising SEQ ID NO:70 and a heavy chain sequence comprising SEQ ID NO:69.

Methods of generating any of the aforementioned anti-αvβ5 antibody variants comprising amino acid substitutions are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985). The antibodies described herein that have increased or reduced effector function can be stabilized by methods described in US2012/0100140 (incorporated by reference herein).

Anti-αvβ5 Antibodies with Altered Glycosylation

Glycan removal produces a structural change that should greatly reduce binding to all members of the Fc receptor family across species. In glycosylated antibodies, including anti-αvβ5 antibodies, the glycans (oligosaccharides) attached to the conserved N-linked site in the CH2 domains of the Fc dimer are enclosed between the CH2 domains, with the sugar residues making contact with specific amino acid residues on the opposing CH2 domain. Different glycosylation patterns are associated with different biological properties of antibodies (Jefferis and Lund, 1997, Chem. Immunol., 65: 111-128; Wright and Morrison, 1997, Trends Biotechnol., 15: 26-32). Certain specific glycoforms confer potentially advantageous biological properties. Loss of the glycans changes spacing between the domains and increases their mobility relative to each other and is expected to have an inhibitory effect on the binding of all members of the Fc receptor family. For example, in vitro studies with various glycosylated antibodies have demonstrated that removal of the CH2 glycans alters the Fc structure such that antibody binding to Fc receptors and the complement protein C1Q are greatly reduced. Another known approach to reducing effector functions is to inhibit production of or remove the N-linked glycans at position 297 (EU numbering) in the CH2 domain of the Fc (Nose et al., 1983 PNAS 80: 6632; Leatherbarrow et al., 1985 Mol. Immunol. 22: 407; Tao et al., 1989 J. Immunol. 143: 2595; Lund et al., 1990 Mol. Immunol. 27: 1145; Dorai et al., 1991 Hybridoma 10:211; Hand et al., 1992 Cancer Immunol. Immunother. 35:165; Leader et al., 1991 Immunology 72: 481; Pound et al., 1993 Mol. Immunol. 30:233; Boyd et al., 1995 Mol. Immunol. 32: 1311). It is also known that different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to Clq protein (Wright A. & Morrison S L. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632).

Importantly, while removal of the glycans in the CH2 domain appears to have a significant effect on effector function, other functional and physical properties of the antibody remains unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

Although there is in vivo validation of the aglycosyl approach, there are reports of residual effector function with aglycosyl mAbs (see, e.g., Pound, J. D. et al. (1993) Mol. Immunol. 30(3): 233-41; Dorai, H. et al. (1991) Hybridoma 10(2): 211-7). Armour et al. show residual binding to FcγRIIa and FcγRIIb proteins (Eur. J. Immunol. (1999) 29: 2613-1624; Mol. Immunol. 40 (2003) 585-593). Thus a further decrease in effector function, particularly complement activation, may be important to guarantee complete ablation of activity in some instances. For that reason, aglycosyl forms of IgG2 and IgG4 and a G1/G4 hybrid are useful in methods and antibody compositions of the invention having reduced effector functions.

The anti-αvβ5 antibodies of the present invention may be modified or altered to elicit reduced effector function(s) (compared to a second αvβ5-specific antibody) while optionally retaining the other valuable attributes of the Fc portion.

Accordingly, in certain embodiments, the present invention relates to aglycosyl anti-αvβ5 antibodies with decreased effector function, which are characterized by a modification at the conserved N-linked site in the CH2 domains of the Fc portion of the antibody. A modification of the conserved N-linked site in the CH2 domains of the Fc dimer can lead to aglycosyl anti-αvβ5 antibodies. Examples of such modifications include mutation of the conserved N-linked site in the CH2 domains of the Fc dimer, removal of glycans attached to the N-linked site in the CH2 domains, and prevention of glycosylation. For example, an aglycosyl anti-αvβ5 antibody may be created by changing the canonical N-linked Asn site in the heavy chain CH2 domain to a Gln residue (see, for example, WO 05/03175 and US 2006-0193856).

In one embodiment of present invention, the modification comprises a mutation at the heavy chain glycosylation site to prevent glycosylation at the site. Thus, in one embodiment of this invention, the aglycosyl anti-αvβ5 antibodies are prepared by mutation of the heavy chain glycosylation site, i.e., mutation of N298Q (N297Q using Kabat numbering) and expressed in an appropriate host cell. For example, this mutation may be accomplished by following the manufacturer's recommended protocol for unique site mutagenesis kit from Amersham-Pharmacia Biotech® (Piscataway, N.J., USA).

The mutated antibody can be stably expressed in a host cell (e. g. NSO or CHO cell) and then purified. As one example, purification can be carried out using Protein A and gel filtration chromatography. It will be apparent to those of skill in the art that additional methods of expression and purification may also be used.

In another embodiment of the present invention, the aglycosyl anti-αvβ5 antibodies have decreased effector function, wherein the modification at the conserved N-linked site in the CH2 domains of the Fc portion of said antibody or antibody derivative comprises the removal of the CH2 domain glycans, i.e., deglycosylation. These aglycosyl anti-αvβ5 antibodies may be generated by conventional methods and then deglycosylated enzymatically. Methods for enzymatic deglycosylation of antibodies are well known to those of skill in the art (Williams, 1973; Winkelhake & Nicolson, 1976 J. Biol Chem. 251:1074-80.).

In another embodiment of this invention, deglycosylation may be achieved by growing host cells which produce the antibodies in culture medium comprising a glycosylation inhibitor such as tunicamycin (Nose & Wigzell, 1983). That is, the modification is the reduction or prevention of glycosylation at the conserved N-linked site in the CH2 domains of the Fc portion of said antibody.

In other embodiments of this invention, recombinant X polypeptides (or cells or cell membranes containing such polypeptides) may be used as an antigen to generate an anti-αvβ5 antibody or antibody derivatives, which may then be deglycosylated.

In alternative embodiments, agyclosyl anti-αvβ5 antibodies or anti-αvβ5 antibodies with reduced glycosylation may be produced by the method described in Taylor et al. (WO 05/18572 and US 2007-0048300). For example, in one embodiment, an anti-αvβ5 aglycosyl antibody may be produced by altering a first amino acid residue (e.g., by substitution, insertion, deletion, or by chemical modification), wherein the altered first amino acid residue inhibits the glycosylation of a second residue by either steric hindrance or charge or both. In certain embodiments, the first amino acid residue is modified by amino acid substitution. In further embodiments, the amino acid substitution is selected from the group consisting of Gly, Ala, Val, Leu, Ile, Phe, Asn, Gln, Trp, Pro, Ser, Thr, Tyr, Cys, Met, Asp, Glu, Lys, Arg, and His. In other embodiments, the amino acid substitution is a non-traditional amino acid residue. The second amino acid residue may be near or within a glycosylation motif, for example, an N-linked glycosylation motif that contains the amino acid sequence NXT or NXS. In one exemplary embodiment, the first amino acid residue is amino acid 299 and the second amino acid residue is amino acid 297, according to the Kabat numbering. For example, the first amino acid substitution may be T299A, T299N, T299G, T299Y, T299C, T299H, T299E, T299D, T299K, T299R, T299G, T299I, T299L, T299M, T299F, T299P, T299W, and T299V, according to the Kabat numbering. In particular embodiments, the amino acid substitution is T299C.

Effector function may also be reduced by modifying an antibody of the present invention such that the antibody contains a blocking moiety. Exemplary blocking moieties include moieties of sufficient steric bulk and/or charge such that reduced glycosylation occurs, for example, by blocking the ability of a glycosidase to glycosylate the polypeptide. The blocking moiety may additionally or alternatively reduce effector function, for example, by inhibiting the ability of the Fc region to bind a receptor or complement protein. In some embodiments, the present invention relates to an αvβ5-binding protein, e.g., an anti-αvβ5 antibody, comprising a variant Fc region, the variant Fc region comprising a first amino acid residue and an N-glycosylation site, the first amino acid residue modified with side chain chemistry to achieve increased steric bulk or increased electrostatic charge compared to the unmodified first amino acid residue, thereby reducing the level of or otherwise altering glycosylation at the N-glycosylation site. In certain of these embodiments, the variant Fc region confers reduced effector function compared to a control, non-variant Fc region. In further embodiments, the side chain with increased steric bulk is a side chain of an amino acid residue selected from the group consisting of Phe, Trp, His, Glu, Gln, Arg, Lys, Met and Tyr. In yet further embodiments, the side chain chemistry with increased electrostatic charge is a side chain of an amino acid residue selected from the group consisting of Asp, Glu, Lys, Arg, and His.

Accordingly, in one embodiment, glycosylation and Fc binding can be modulated by substituting T299 with a charged side chain chemistry such as D, E, K, or R. The resulting antibody will have reduced glycosylation as well as reduced Fc binding affinity to an Fc receptor due to unfavorable electrostatic interactions.

In another embodiment, a T299C variant antibody, which is both aglycosylated and capable of forming a cysteine adduct, may exhibit less effector function (e.g., FcγRI binding) compared to its aglycosylated antibody counterpart (see, e.g., WO 05/18572). Accordingly, alteration of a first amino acid proximal to a glycosylation motif can inhibit the glycosylation of the antibody at a second amino acid residue; when the first amino acid is a cysteine residue, the antibody may exhibit even further reduced effector function. In addition, inhibition of glycosylation of an antibody of the IgG4 subtype may have a more profound effect on FcγRI binding compared to the effects of agycosylation in the other subtypes.

In additional embodiments, the present invention relates to anti-αvβ5 antibodies with altered glycosylation that exhibit reduced binding to one or more FcR receptors and that optionally also exhibit increased or normal binding to one or more Fc receptors and/or complement—e.g., antibodies with altered glycosylation that at least maintain the same or similar binding affinity to one or more Fc receptors and/or complement as a native, control anti-αvβ5 antibody). For example, anti-αvβ5 antibodies with predominantly Man₅GlcNAc₂N-glycan as the glycan structure present (e.g., wherein Man₅GlcNAc₂N-glycan structure is present at a level that is at least about 5 mole percent more than the next predominant glycan structure of the Ig composition) may exhibit altered effector function compared to an anti-αvβ5 antibody population wherein Man₅GlcNAc₂N-glycan structure is not predominant Antibodies with predominantly this glycan structure exhibit decreased binding to FcγRIIa and FcγRIIb, increased binding to FcγRIIIa and FcγRIIIb, and increased binding to Clq subunit of the C1 complex (see US 2006-0257399). This glycan structure, when it is the predominant glycan structure, confers increased ADCC, increased CDC, increased serum half-life, increased antibody production of B cells, and decreased phagocytosis by macrophages.

In general, the glycosylation structures on a glycoprotein will vary depending upon the expression host and culturing conditions (Raju, T S. BioProcess International April 2003. 44-53). Such differences can lead to changes in both effector function and pharmacokinetics (Israel et al. Immunology, 1996; 89(4):573-578; Newkirk et al. P. Clin. Exp., 1996; 106(2):259-64). For example, galactosylation can vary with cell culture conditions, which may render some immunoglobulin compositions immunogenic depending on their specific galactose pattern (Patel et al., 1992. Biochem J. 285: 839-845). The oligosaccharide structures of glycoproteins produced by non-human mammalian cells tend to be more closely related to those of human glycoproteins. Further, protein expression host systems may be engineered or selected to express a predominant Ig glycoform or alternatively may naturally produce glycoproteins having predominant glycan structures. Examples of engineered protein expression host systems producing a glycoprotein having a predominant glycoform include gene knockouts/mutations (Shields et al., 2002, JBC, 277: 26733-26740); genetic engineering in (Umana et al., 1999, Nature Biotech., 17: 176-180) or a combination of both. Alternatively, certain cells naturally express a predominant glycoform—for example, chickens, humans and cows (Raju et al., 2000, Glycobiology, 10: 477-486). Thus, the expression of an anti-αvβ5 antibody or antibody composition having altered glycosylation (e.g., predominantly one specific glycan structure) can be obtained by one skilled in the art by selecting at least one of many expression host systems. Protein expression host systems that may be used to produce anti-αvβ5 antibodies of the present invention include animal, plant, insect, bacterial cells and the like. For example, US 2007-0065909, 2007-0020725, and 2005-0170464 describe producing aglycosylated immunoglobulin molecules in bacterial cells. As a further example, Wright and Morrison produced antibodies in a CHO cell line deficient in glycosylation (1994 J Exp Med 180: 1087-1096) and showed that antibodies produced in this cell line were incapable of complement-mediated cytolysis. Other examples of expression host systems found in the art for production of glycoproteins include: CHO cells: Raju WO 99/22764 and Presta WO 03/35835; hybridoma cells: Trebak et al., 1999, J. Immunol. Methods, 230: 59-70; insect cells: Hsu et al., 1997, JBC, 272:9062-970, and plant cells: Gerngross et al., WO 04/74499. To the extent that a given cell or extract has resulted in the glycosylation of a given motif, art recognized techniques for determining if the motif has been glycosylated are available, for example, using gel electrophoresis and/or mass spectroscopy.

Additional methods for altering glycosylation sites of antibodies are described, e.g., in U.S. Pat. No. 6,350,861 and U.S. Pat. No. 5,714,350, WO 05/18572 and WO 05/03175; these methods can be used to produce anti-αvβ5 antibodies of the present invention with altered, reduced, or no glycosylation.

The aglycosyl anti-αvβ5 antibodies with reduced effector function may be antibodies that comprise modifications or that may be conjugated to comprise a functional moiety. Such moieties include a blocking moiety (e.g., a PEG moiety, cysteine adducts, etc.), a detectable moiety (e.g., fluorescent moieties, radioisotopic moieties, radiopaque moieties, etc., including diagnostic moieties), a therapeutic moiety (e.g., cytotoxic agents, anti-inflammatory agents, immunomodulatory agents, anti-infective agents, anti-cancer agents, anti-neurodegenerative agents, radionuclides, etc.), and/or a binding moiety or bait (e.g., that allows the antibody to be pre-targeted to a tumor and then to bind a second molecule, composed of the complementary binding moiety or prey and a detectable moiety or therapeutic moiety, as described above).

### Indications

The anti-αvβ5 antibodies or antigen-binding fragments thereof described herein block αvβ5 and inhibit vascular permeability in response to inflammation and injury. In addition, these antibodies or antigen-binding fragments can inhibit endothelial migration. Furthermore, these antibodies or antigen-binding fragments can inhibit TGF-β activation and fibrosis. These antibodies or antigen-binding fragments thereof can be used to treat, prevent, or reduce the symptoms or severity of a wide range of diseases or conditions. Such diseases or conditions include acute kidney injury, acute lung injury, stroke (cerebral hemorrhage), acute respiratory distress syndrome, asthma, pulmonary edema, lung fibrosis (e.g., idiopathic pulmonary fibrosis (IPF), usual interstitial pneumonia (UIP)), sepsis, myocardial infarction, cancer (e.g., pancreatic cancer, lung cancer, breast cancer, colorectal cancer, head and neck cancer, esophageal cancer, skin cancer, prostate cancer, cervical cancer, colon cancer, ovarian cancer, and endometrial cancer), dyslipidemias, obesity, and ocular neovascularization disease.

In certain embodiments, the antibodies or antigen-binding fragments thereof described herein can be used to treat or reduce the symptoms or severity of acute lung injury. In certain embodiments, the antibodies or antigen-binding fragments thereof described herein can be used to treat or reduce the symptoms or severity of pulmonary edema (e.g., edema associated with lung injury). In certain embodiments, the antibodies or antigen-binding fragments thereof described herein can be used to treat or reduce the symptoms or severity of sepsis. In some embodiments, the antibodies or antigen-binding fragments thereof described herein can be used to treat lung fibrosis (e.g., IPF, UIP). In other embodiments, the antibodies or antigen-binding fragments thereof described herein can be used to protect against epithelial and/or endothelial cell injury. In certain embodiments, the antibodies or antigen-binding fragments thereof described herein can be used to reduce or prevent alveolar epithelial injury. In yet other embodiments, the antibodies or antigen-binding fragments thereof described herein can be used to treat epithelial cancers (e.g., head and neck (including oral, laryngeal, pharyngeal, esophageal), breast, lung, prostate, cervical, colon, pancreatic, skin (basal cell carcinomas) and ovarian cancers). In some embodiments, the antibodies or antigen-binding fragments thereof described herein can be used as anti-angiogenic agents. In further embodiments, the antibodies or antigen-binding fragments thereof described herein can be used to block interaction of the αvβ5 receptor with RGD-containing ligands, e.g., proteins on the surface of viruses or other pathogens, thereby reducing or preventing infection.

The efficacy of the antibodies of the invention can be assessed in various animal models that are well-known in the art. Animal models for acute lung injury include: the pulmonary ischemia/reperfusion model (Sakuma T. et al., Am J Physiol Lung Cell Mol Physiol., 276: L137-L145, (1999); WO 2005/094391); the non-pulmonary ischemia/reperfusion model (Koike K. et al., J Surg Res., 52:656-662 (1992)); the oleic acid model (Schuster, Am J Respir Crit Care Med, 149: 245-260 (1994)); the LPS model (Wiener-Kronish et al., J Clin Invest, 88: 864-875 (1991); the acid aspiration model (Modelska K. et al., Am J Respir Crit Care Med, 160: 1450-1456, (1999)); the hyperoxia model (Frank L. et al., J Appl Physiol., 45: 699-704 (1978)); the bleomycin model (Moore et al., Am J Physiol Lung Cell Mol Physiol., 294: L152-L160 (2008)); the saline lavage model (Lachmann B. et al., Acta Anaesthesiol Scand., 24: 231-236, (1980)); the cecal ligation and puncture model (Villar J. et al., Crit Care Med., 22: 914-921 (1994)); and the intrapulmonary bacteria model (Fox-Dewhurst R. et al., Am J Respir Crit Care Med., 155: 2030-2040 (1997). Mouse models for lung fibrosis include bleomycin- (Pittet et al., J. Clin. Invest., 107(12):1537-1544 (2001); and Munger et al., Cell, 96:319-328 (1999)) and irradiation-inducible lung fibrosis (Franko et al., Rad. Res., 140:347-355 (1994)). Animal models for sepsis are known in the art and include toxaemia models (e.g., LPS injection), bacterial infection models, host-barrier disruption models (Doi et al., J. Clin. Invest., 119(10):2868-2878 (2009); WO 2011/011775). Finally, the αvβ5 antibodies described herein can be assessed for their ability to inhibit tumor growth, progression, and metastasis in standard in vivo tumor growth and metastasis models. See, e.g., Rockwell et al., J. Natl. Cancer Inst., 49:735 (1972); Guy et al., Mol. Cell Biol., 12:954 (1992); Wyckoff et al., Cancer Res., 60:2504 (2000); and Oft et al., Curr. Biol., 8:1243 (1998).

The efficacy of treatments may be measured by a number of available diagnostic tools, including physical examination, blood tests, pulmonary function tests, observation and scoring of scarring or fibrotic lesions, deposition of extracellular matrix such as collagen, smooth muscle actin and fibronectin, ultrasound, magnetic resonance imaging (MRI), and CT scan.

### Pharmaceutical Compositions

An anti-αvβ5 antibody or antigen-binding fragment thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, an anti-αvβ5 antibody described herein is formulated with excipient materials, such as sodium citrate, sodium dibasic phosphate heptahydrate, sodium monobasic phosphate, Tween-80, and a stabilizer. It can be provided, for example, in a buffered solution at a suitable concentration and can be stored at 2-8° C. In some other embodiments, the pH of the composition is between about 5.5 and 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5).

The pharmaceutical compositions can also include agents that reduce aggregation of the αvβ5 antibody or antigen-binding fragment thereof when formulated. Examples of aggregation reducing agents include one or more amino acids selected from the group consisting of methionine, arginine, lysine, aspartic acid, glycine, and glutamic acid. These amino acids may be added to the formulation to a concentration of about 0.5 mM to about 145 mM (e.g., 0.5 mM, 1 mM, 2 mM, 5 mM, 10 mM, 25 mM, 50 mM, 100 mM). The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

Such compositions can be administered by a parenteral mode (e.g., intravenous, subcutaneous, intraperitoneal, or intramuscular injection). In one embodiment, the anti-αvβ5 antibody or antigen-binding fragment thereof compositions are administered subcutaneously. In one embodiment, the anti-αvβ5 antibody or antigen-binding fragment thereof compositions are administered intravenously. The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, intranasal, oral inhalation, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-αvβ5 antibody or antigen-binding fragment thereof may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In one embodiment, the pharmaceutical formulation comprises an anti-αvβ5 antibody or antigen-binding fragment thereof at a concentration of about 0.5 mg/mL to 500 mg/mL (e.g., 0.5 mg/mL, 1 mg/mL, 5 mg/mL, 10 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 250 mg/mL, 300 mg/mL, 350 mg/mL, 400 mg/mL, 450 mg/mL, 500 mg/mL), formulated with a pharmaceutically acceptable carrier. In some embodiments, the anti-αvβ5 antibody or antigen-binding fragment thereof is formulated in sterile distilled water or phosphate buffered saline. The pH of the pharmaceutical formulation may be between 5.5 and 7.5 (e.g., 5.5, 5.6, 5.7, 5.8, 5.9, 6.0, 6.1, 6.2 6.3, 6.4 6.5, 6.6 6.7, 6.8, 6.9 7.0, 7.1, 7.3, 7.4, 7.5).

### Administration

The anti-αvβ5 antibody or antigen-binding fragment thereof can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, intranasal, oral inhalation, epidural, and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

The antibody or antigen-binding fragment thereof can be administered as a fixed dose, or in a mg/kg dose. The dose can also be chosen to reduce or avoid production of antibodies against the anti-αvβ5 antibody. Dosage regimens are adjusted to provide the desired response, e.g., a therapeutic response or a combinatorial therapeutic effect. Generally, doses of the anti-αvβ5 antibody or antigen binding fragment thereof (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In certain embodiments, a subject in need of treatment with an anti-αvβ5 antibody or antigen binding fragment thereof is administered the antibody at a dose of 1 mg/kg to 30 mg/kg. In some embodiments, a subject in need of treatment with an anti-αvβ5 antibody or antigen-binding fragment thereof is administered the antibody at a dose of 1 mg/kg, 2 mg/kg, 4 mg/kg, 5 mg/kg, 7 mg/kg 10 mg/kg, 12 mg/kg, 15 mg/kg, 20 mg/kg, 25 mg/kg, 28 mg/kg, 30 mg/kg, 35 mg/kg, 40 mg/kg, or 50 mg/kg. In certain embodiments, a subject in need of treatment with a toxin-conjugated anti-αvβ5 antibody or antigen binding fragment thereof is administered the toxin-conjugated antibody or antigen binding fragment thereof at a dose of 0.1 mg/kg to 30 mg/kg. In some embodiments, a subject in need of treatment with a toxin-conjugated anti-αvβ5 antibody or antigen-binding fragment thereof is administered the toxin-conjugated antibody or antigen binding fragment thereof at a dose of 0.1 mg/kg, 0.2 mg/kg, 0.3 mg/kg, 0.4 mg/kg, 0.5 mg/kg, 0.6 mg/kg, 0.7 mg/kg, 0.75 mg/kg, 0.8 mg/kg, 0.9 mg/kg, 1 mg/kg, 2 mg/kg, 4 mg/kg, 5 mg/kg, 7 mg/kg 10 mg/kg, 12 mg/kg, 15 mg/kg, 20 mg/kg, 25 mg/kg, 28 mg/kg, 30 mg/kg, 35 mg/kg, 40 mg/kg, or 50 mg/kg. In a specific embodiment, the antibodies or antigen-binding fragments thereof are administered subcutaneously at a dose of 1 mg/kg to 3 mg/kg. In another embodiment, the antibodies or antigen-binding fragments thereof are administered intravenously at a dose of 4 mg/kg to 30 mg/kg. In certain embodiments, the toxin-conjugated versions of the antibodies or antigen-binding fragments thereof are administered intravenously at a dose of 0.1 mg/kg to 30 mg/kg.

A composition may comprise about 1 mg/mL to 100 mg/ml or about 10 mg/mL to 100 mg/ml or about 50 to 250 mg/mL or about 100 to 150 mg/ml or about 100 to 250 mg/ml of anti-αvβ5 antibody or an antigen-binding fragment thereof. In certain embodiments, the anti-αvβ5 antibody or antigen-binding fragment thereof in a composition is predominantly in monomeric form, e.g., at least about 90%, 92%, 94%, 96%, 98%, 98.5% or 99% in monomeric form. Certain anti-αvβ5 antibody or antigen-binding fragment thereof compositions may comprise less than about 5, 4, 3, 2, 1, 0.5, 0.3 or 0.1% aggregates, as detected, e.g., by UV at A280 nm. Certain anti-αvβ5 antibody or antigen-binding fragment thereof compositions comprise less than about 5, 4, 3, 2, 1, 0.5, 0.3, 0.2 or 0.1% fragments, as detected, e.g., by UV at A280 nm.

Dosage unit form or “fixed dose” as used herein refers to physically discrete units suited as unitary dosages for the subjects to be treated; each unit contains a predetermined quantity of anti-αvβ5 antibody calculated to produce the desired therapeutic effect in association with the required pharmaceutical carrier and optionally in association with the other agent. Single or multiple dosages may be given. Alternatively, or in addition, the antibody may be administered via continuous infusion.

An anti-αvβ5 antibody or antigen-binding fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

If a subject is at risk for developing a disorder described herein, the antibody can be administered before the full onset of the disorder, e.g., as a preventative measure. The duration of such preventative treatment can be a single dosage of the antibody or the treatment may continue (e.g., multiple dosages). For example, a subject at risk for the disorder or who has a predisposition for the disorder may be treated with the antibody for days, weeks, months, or even years so as to prevent the disorder from occurring or fulminating.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In certain embodiments, the anti-αvβ5 antibody or antigen-binding fragment thereof is administered subcutaneously at a concentration of about 1 mg/mL to about 500 mg/mL (e.g., 1 mg/mL, 2 mg/mL, 3 mg/mL, 4 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 400 mg/mL, 450 mg/mL). In one embodiment, the anti-αvβ5 antibody or antigen-binding fragment thereof is administered subcutaneously at a concentration of 50 mg/mL. In another embodiment, the anti-αvβ5 antibody or antigen-binding fragment thereof is administered intravenously at a concentration of about 1 mg/mL to about 500 mg/mL. In a particular embodiment, the anti-αvβ5 antibody or antigen-binding fragment thereof is administered intravenously at a concentration of 50 mg/mL.

The anti-αvβ5 antibody or antigen-binding fragment thereof can be administered to a patient in need thereof (e.g., a patient with lung fibrosis) in combination with a second therapeutic agent. The second therapeutic agent depends on the type of disease or disorder being treated. For example, the second therapeutic agent can be an antagonist (e.g., antibodies, polypeptide antagonists, and/or small molecule antagonists) of one or more: other integrin receptors (e.g., α1β1, α4β1, αvβ8, αvβ6, αvβ1, etc.); cytokines (e.g., TGF-β, IL-4, IL-13, IL-17); chemokines (e.g., CCL2, CXCL8, CXCL12); growth factors (e.g., Connective tissue growth factor (CTGF), Platelet-derived growth factor (PDGF), Vascular endothelial growth factor (VEGF), Fibroblast growth factor (FGF), Insulin-like growth factor-1 (IGF-1)), and/or small secreted signaling proteins (e.g., Wnt proteins, endothelin-1). The anti-αvβ5 antibody or antigen-binding fragment thereof and the second therapeutic agent may be administered simultaneously or sequentially. In certain embodiments, the anti-αvβ5 antibody or antigen-binding fragment thereof and the second therapeutic agent can each be administered at either a subtherapeutic dose or a therapeutic dose.

In certain embodiments, where a patient has, or is at risk of developing acute lung injury, pulmonary edema, or ARDS, the anti-αvβ5 antibody or antigen-binding fragment thereof can be administered in combination with diuretic agents, bronchodilating agents, narcotics, oxygen, and selective tourniquet application. In addition, the anti-αvβ5 integrin antibodies disclosed herein may be administered in conjunction with a second therapeutic agent that targets metabolic pathways that are implicated in acute lung injury, ARDS, or PE. For example, an anti-αvβ5 integrin antibody or antigen-binding fragment thereof may be administered in conjunction with TGFβ pathway inhibitors, activated Protein C, steroids, GM-CSF, platelet inhibitors, β-2 agonists, surfactants, other antibodies that specifically bind to αvβ5 integrin or β5, a second antagonist of αvβ5 integrin, antibodies that specifically bind to a αvβ6 integrin, antagonists of αvβ6 integrin, thrombin receptor antagonists, anti-thrombin agents, rho kinase inhibitors, and nucleic acids that inhibit expression of αvβ5 integrin including e.g., the antisense oligonucleotides, ribozymes, miRNA, and siRNA. Suitable TGFβ pathway inhibitors include, e.g., TGF-β antibodies (including those that specifically block TGF-β 1, TGF-β2, TGF-β3 or any combination thereof) as described in e.g., Ling et al., J. Amer. Soc. Nephrol., 14: 377-388 (2003), McCormick et al, J. Immunol., 163:5693-5699 (1999), and Cordeiro, Curr. Opin. Mol. Ther., 5(2): 199-203 (2003); TGF-β receptor type II inhibitors or TGF-3 receptor type I kinase inhibitors as described in, e.g., DaCosta Bayfield, Mol. Pharmacol., 65(3):744-52 (2004), Laping, Curr. Opin. Pharmacol., 3(2):204-8 (2003), Laping, Mol. Pharmacol., 62(1):58-64 (2002); soluble TGF-β receptor type II as described in, e.g., Pittet, J. Clin. Invest., 107:1537-1544 (2001); Wang et al, Exp Lung Res., 28(6):405-17 (2002) and Wang, Thorax, 54(9):805-12 (1999); soluble latency associated peptides as described in, e.g., Zhang, J Invest. Dermatol., 121(4):713-9 (2003); thrombospondin I inhibitors as described in, e.g., Crawford et al, Cell, 93:1159-1170 (1998), Riberiro et al, J. Biol. Chem., 274:13586-13593 (1999), and Schultz-Cherry et al, J. Biol. Chem., 269: 26775-26782 (1994). Suitable β-2 agonists include, e.g., albuterol, bitolterol, formoterol, isoproterenol, levalbuterol, metaproterenol, pirbuterol, salmeterol, and terbutaline. Suitable surfactants include, e.g., exosurf, infasurf, KL-4, pumactant, survanta, venticute, and surfactant TA, as described in Taeusch et al, Acta Pharmacol Sin 23 Supplement: 11-15 (2002). Suitable anti-thrombin agents include, e.g., hirudin, Hirulog (Biogen), argatroban, efegatran, and compounds described in U.S. Pat. No. 6,518,244. Suitable thrombin receptor antagonists are described in, e.g., U.S. Pat. Nos. 6,544,982; 6,515,023; 6,403,612; 6,399,581; and 5,446,131. Suitable rho kinase inhibitors include, e.g., Y-27632 as described in e.g., Tasaka et al, Am J Respir Cell Mol Biol., 32(6):504-10 (2005); fasudil as described in, e.g., Nishikimi et al, J Hypertens., 22(9):1787-96 (2004); 1-(5-isoquinolinesulfonyl)-homopiperazine (HA-1077), (S)-(+)-2-methyl-1-[(4-methyl-5-isoquinoline)sulfonyl]-homopiperazine (H-1 152P) as described in Sasaki et al, Pharmacol Ther., 93(2-3):225-32 (2002), and additional rho kinase inhibitors as described in, e.g., U.S. Pat. Nos. 6,451,825 and 6,218,410 and U.S. Patent Publication Nos. 20050014783 and 20030134775. In addition, the antagonist of αvβ5 integrin may be administered combination with an adenovirus expressing ATPase as described in U.S. Patent Application No. 20020192186; with a β2 adrenergic receptor as described in U.S. Patent Application No. 20020004042; with VEGFβ antagonists as described in U.S. Pat. No. 6,284,751; with lipid peroxidation inhibitors as described in U.S. Pat. No. 5,231,114; and with small molecule inhibitors for αvβ6, αvβ5, and αvβ3 integrins as described in, e.g., US Patent Application Nos. 2000/40019206, 2004/0019037, 2004/0019035, 2004/0018192, 2004/0010023, 2003/0181440, 2003/0171271, 2003/0139398, 2002/0037889, 2002/0077321, 2002/0072500, U.S. Pat. No. 6,683,051 and Goodman et al., J. Med Chem. 45(5): 1045-51 (2002).

In certain embodiments, where a patient has, or is at risk of developing sepsis, the anti-αvβ5 antibody or antigen-binding fragment thereof can be administered in combination with any of the standard treatments for sepsis including, e.g., antibiotics, statins, steroids, activated Protein C, diuretic agents, vasoconstrictors, or inotropic drugs. Antibiotic therapies are common, and can best be selected by the medical professional to specifically target a particular infection. Exemplary antibiotics include, e.g., penicillin, erythromycin, cyclic lipopeptides (daptomycin), glycylcyclines (tigecycline), and oxazolidinones (linezolid). Statins (HMG-CoA reductase inhibitors) include, e.g., simvastatin or atorvastatin. In addition, an anti-αvβ5 integrin antibody or antigen binding fragment thereof may be administered in conjunction with agents that target metabolic pathways that are implicated in sepsis. For example, an antagonist of αvβ5 integrin may be administered in conjunction with TGFβ pathway inhibitors, activated Protein C, GM-CSF, antibodies that specifically bind to αvβ5 integrin or β5, a second antagonist of αvβ5 integrin, antibodies that specifically bind to a αvβ6 integrin, antagonists of αvβ6 integrin, thrombin receptor antagonists, anti-thrombin agents, rho kinase inhibitors, and nucleic acids that inhibit expression of αvβ5 integrin including e.g., antisense oligonucleotides, ribozymes, siRNA, microRNA.

### Devices and Kits for Therapy

Pharmaceutical compositions that include the anti-αvβ5 antibody or antigen-binding fragment thereof can be administered with a medical device. The device can be designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include anti-αvβ5 antibody or antigen-binding fragment thereof, and can be configured to deliver one or more unit doses of the antibody. The device can be further configured to administer a second agent, e.g., a chemo therapeutic agent, either as a single pharmaceutical composition that also includes the anti-αvβ5 antibody or antigen-binding fragment thereof or as two separate pharmaceutical compositions.

The pharmaceutical composition may be administered with a syringe. The pharmaceutical composition can also be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An anti-αvβ5 antibody or antigen-binding fragment thereof can be provided in a kit. In one embodiment, the kit includes (a) a container that contains a composition that includes anti-αvβ5 antibody, and optionally (b) informational material. The informational material can be descriptive, instructional, marketing or other material that relates to the methods described herein and/or the use of the agents for therapeutic benefit.

In an embodiment, the kit also includes a second therapeutic agent for treating a disorder described herein (e.g., an antagonist (e.g., antibodies, polypeptide antagonists, and/or small molecule antagonists) of one or more: other integrin receptors (e.g., α1β1, α4β1, αvβ8, αvβ5, αvβ1, etc.); cytokines (e.g., TGF-β, IL-4, IL-13, IL-17); chemokines (e.g., CCL2, CXCL8, CXCL12); growth factors (e.g., Connective tissue growth factor (CTGF), Platelet-derived growth factor (PDGF), Vascular endothelial growth factor (VEGF), Fibroblast growth factor (FGF), Insulin-like growth factor-1 (IGF-1)), small secreted signaling proteins (e.g., Wnt proteins, endothelin-1) a steroid, a cytotoxic compound, a radioisotope, a prodrug-activating enzyme, colchicine, oxygen, an antioxidant (e.g., N-acetylcysteine), a metal chelator (e.g., terathiomolybdate), IFN-β, IFN-γ, alpha-antitrypsin). For example, the kit includes a first container that contains a composition that includes the anti-αvβ5 antibody, and a second container that includes the second therapeutic agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-αvβ5 antibody or antigen-binding fragment thereof, e.g., in a suitable dose, dosage form, or mode of administration (e.g., a dose, dosage form, or mode of administration described herein), to treat a subject who has had or who is at risk for an immunological disorder described herein. The information can be provided in a variety of formats, include printed text, computer readable material, video recording, or audio recording, or information that provides a link or address to substantive material, e.g., on the internet.

In addition to the antibody, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. In certain embodiments, the antibody or antigen binding fragment thereof in the liquid solution is at a concentration of about 25 mg/mL to about 250 mg/mL (e.g., 40 mg/mL, 50 mg/mL, 60 mg/mL, 75 mg/mL, 85 mg/mL, 100 mg/mL, 125 mg/mL, 150 mg/mL, 200 mg/mL). When the antibody or antigen binding fragment is provided as a lyophilized product, the antibody or antigen binding fragment is at about 75 mg/vial to about 200 mg/vial (e.g., 100 mg/vial, 125 mg/vial, 150 mg/vial). The lyophilized powder is generally reconstituted by the addition of a suitable solvent. The solvent, e.g., sterile water or buffer (e.g., PBS), can optionally be provided in the kit. In certain embodiments, the lyophilized product is at about 100 mg/vial and reconstituted to a liquid solution at a concentration of 75 mg/mL.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-αvβ5 antibody or antigen-binding fragment thereof and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### Diagnostic Uses

Anti-αvβ5 antibodies or antigen-binding fragments thereof can be used in a diagnostic method for detecting the presence of αvβ5 in vitro or in vivo (e.g., in vivo imaging in a subject). For example, anti-αvβ5 antibodies can be administered to a subject to detect αvβ5 within the subject. For example, the antibody can be labeled, e.g., with an MRI detectable label or a radiolabel. The subject can be evaluated using a means for detecting the detectable label. For example, the subject can be scanned to evaluate localization of the antibody within the subject. For example, the subject is imaged, e.g., by NMR or other tomographic means.

Examples of labels useful for diagnostic imaging include radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, can also be employed. The protein ligand can be labeled with such reagents using known techniques. For example, see Wensel and Meares (1983) Radioimmunoimaging and Radioimmunotherapy, Elsevier, New York for techniques relating to the radiolabeling of antibodies and Colcher et al. (1986) Meth. Enzymol. 121: 802-816.

The subject can be “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EPO 502 814 A. Generally, the differences related to relaxation time constants T1 and T2 of water protons in different environments are used to generate an image. However, these differences can be insufficient to provide sharp high resolution images.

The differences in these relaxation time constants can be enhanced by contrast agents. Examples of such contrast agents include a number of magnetic agents, paramagnetic agents (which primarily alter T1) and ferromagnetic or superparamagnetic agents (which primarily alter T2 response). Chelates (e.g., EDTA, DTPA and NTA chelates) can be used to attach (and reduce toxicity) of some paramagnetic substances (e.g., Fe³⁺, Mn²⁺, Gd³⁺). Other agents can be in the form of particles, e.g., less than 10 μm to about 10 nm in diameter). Particles can have ferromagnetic, anti-ferromagnetic or superparamagnetic properties. Particles can include, e.g., magnetite (Fe₃O₄), γ-Fe₂O₃, ferrites, and other magnetic mineral compounds of transition elements. Magnetic particles may include one or more magnetic crystals with and without nonmagnetic material. The nonmagnetic material can include synthetic or natural polymers (such as sepharose, dextran, dextrin, starch and the like).

The anti-αvβ5 antibodies or antigen-binding fragments thereof can also be labeled with an indicating group containing the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image αvβ5 distribution.

In another aspect, the disclosure provides a method for detecting the presence of αvβ5 in a sample in vitro (e.g., a biological sample, such as serum, plasma, tissue, biopsy). This method can be used to diagnose a disorder, e.g., acute lung injury, lung fibrosis, or cancer (e.g., pancreatic, lung, breast, colorectal, head and neck, esophageal, skin, or endometrial). The method includes: (i) contacting the sample or a control sample with the anti-αvβ5 antibody; and (ii) evaluating the sample for the presence of αvβ5, e.g., by detecting formation of a complex between the anti-αvβ5 antibody and αvβ5, or by detecting the presence of the antibody or αvβ5. For example, the antibody can be immobilized, e.g., on a support, and retention of the antigen on the support is detected, and/or vice versa. The antibody used may be labeled e.g., with a fluorophore. A control sample can be included. The positive control can be a sample known to have the disease or disorder being assessed, and a negative control can be a sample from a subject who does not have the disease or disorder being assessed. A statistically significant change in the formation of the complex in the sample relative to the control sample can be indicative of the presence of αvβ5 in the sample. Generally, an anti-αvβ5 antibody can be used in applications that include fluorescence polarization, microscopy, ELISA, centrifugation, chromatography, and cell sorting (e.g., fluorescence activated cell sorting). In certain embodiments, the anti-αvβ5 antibody is a humanized ALULA antibody or an antigen-binding fragment thereof. The tissue sample can be, e.g., skin biopsies from human patients with cancer, e.g., pancreatic, lung, breast, colorectal, head and neck, esophageal, skin, or endometrial.

### Examples

### Example 1: Role of Effector Function on In Vivo Efficacy of ALULA

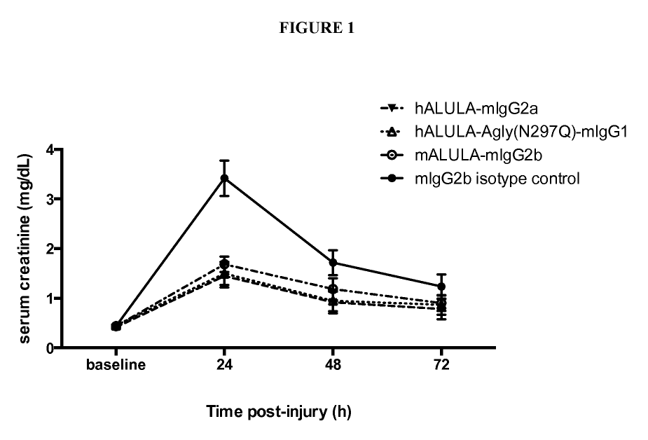

In order to assess the role that Fc effector function (mediated through binding to Fc-gamma receptors and/or complement Clq) plays in the in vivo efficacy of ALULA, the rat ischemia-reperfusion model was carried out using constructs with different murine Fc domains. Two chimeric variants were generated comprising a humanized ALULA variable domain heavy chain sequence (referred to here as “Design-Reference H1”) fused to either mIgG2a (highest effector function) or mIgG1 (N297Q) Agly (lowest effector function) constant domains. These heavy chains were paired with a chimeric light chain comprising a humanized variable domain light chain sequence (referred to here as “Design-Reference L1”) fused to a murine kappa constant domain. Design-Reference H1 and Design-Reference L1 paired together is referred to as the reference humanized αvβ5 design. The two chimeric mAbs described above were tested in the rat ischemia-reperfusion model in parallel with the original ALULA IgG2b construct, and a murine IgG2b isotype control. As shown in FIG. 1, all three ALULA antibodies significantly reduced serum creatinine levels relative to the isotype control, and no significant differences were observed among the three different forms of ALULA. These data indicate that reducing effector function does not affect the efficacy of αvβ5 targeted antibody therapy.

### Example 2: Humanized ALULA Designs

The CDRs of the mature murine ALULA antibody (CDR-H1, CDR-H2 and CDR-H3 for the heavy chain region and CDR-L1, CDR-L2, and CDR-L3 for the light chain region) were grafted onto human acceptor frameworks, based on human germlines humIGHV3-15 and humIGKV1-12, to create CDR-grafted chains, VH0 and VL0, respectively. Six additional heavy chain regions (VH1 to 6) and four additional light chain regions (VL1 to 4) were created by combining several mutations in the human acceptor frameworks of the CDR grafts (discussed below) compared to the CDR-grafted chains. The majority of the mutations that were made in the human acceptor frameworks were backmutations to the amino acid of the mature murine framework to help maintain the structure of the ALULA mature murine CDRs.

Designs VH1 to VH5 and VL1 to VL3 are based on the CDR grafts, i.e., all mature ALULA CDRs grafted onto the human acceptor frameworks. Designs VH6 and VL4, however, contain both CDR1 and CDR3 of ALULA while CDR2 is maintained from the human acceptor framework. The second CDR of ALULA did not mature from the parent murine germline to the mature murine which suggests that the second CDR region may not contact the antigen, therefore the second CDR may not be necessary and that CDR was replaced with the human acceptor sequence to reduce the immunogenic risk of exposed murine sequences.

### Mutations in Human Acceptor Frameworks:

### 1. Heavy Chain

### 1.1 Mutations in VH1, VH2, VH3, VH4, VH5, VH6

The three mutations that were made in the above-listed VH regions are discussed below.

First, a framework mutation R71A was made to make room for mature murine CDR residues Y32 and/or P52a. It is solvated on one side and A at this position is common in human (A 151/544, R 227/544 are the frequencies of these amino acids in a database of 544 human heavy chain sequences), so this mutation should have no immunogenicity risk.

The second mutation, D73T, is a Haidar position for CDR-H2. Solvated on one side, this amino acid contacts CDR-H1 and CDR-H2. T at this position is common in human (T 164/544, N 164/544, K 114/544, D 39/544 are the frequencies of these amino acids in a database of 544 human heavy chain sequences), so this mutation has a very low immunogenicity risk.

The third mutation, L4V is hypermutated from L in the murine germline to V in mature murine. V at this position is uncommon in mouse (V 13/943 vs. L 927/943) and in human (V 9/544 vs. L 529/544 are the frequencies of these amino acids in a database of 544 human heavy chain sequences), but this sidechain is buried, so the immunogenicity risk of this mutation is low. The L4V mutation could affect CDR-H3 structure, and improve the fit with hypermutation T94 (which is T in the acceptor as well).

### 1.2 Mutations in VH2, VH3, VH4, VH5

The three mutations that were made in the above-listed VH regions are discussed below.

First, R66K is a Haidar position for CDR-H2. The K is solvated on one side but has many salt bridges to make to framework residues, presumably to hold its end of CDR-H2 in place. After the CDR graft, R66 can interact similarly, but possibly alter the CDR structure. K at this position is common in human (K 176/544, R 347/544 are the frequencies of these amino acids in a database of 544 human heavy chain sequences).

The second mutation, L78A is a Haidar position support for CDR-H2. This position is buried and contacts CDR-H2. Neighbors that differ in mature murine vs. acceptor are P52a in CDR-H2 and 71, which has been mutated to R71A. The L78A mutation along with R71A should pose no additional risks, and this will help the R71A achieve its mature murine conformation. A at this position is common for human (A 214/544 vs. L 163/544 are the frequencies of these amino acids in a database of 544 human heavy chain sequences), and it is buried, so there is no immunogenicity risk with this mutation.

The third mutation, R38K is semi-buried amongst framework. The combination of F63 in CDR-H2 and R here, created upon CDR grafting, may be crowded, affecting CDR structure. R38K could help retain mature CDR conformation. K here is quite common in human (K 153/544, R 385/544 are the frequencies of these amino acids in a database of 544 human heavy chain sequences). K here will likely still form salt bridges as it usually does.

### 1.3 Mutation in VH4, VH5

The mutation that was made in the above listed VH regions is K75P. This position is solvated and located around a corner from CDR-H2. This position was hypermutated from A to P. P is not observed at this position in human (P 0/544 vs. K 225/544, S 142/544, T 56/544 are the frequencies of these amino acids in a database of 544 human heavy chain sequences) (as well as in mouse), so this mutation has a risk of immunogenicity. The fact of its hypermutation means that it may be important for antigen binding, though based on its location, it is unlikely to contact antigen. Its loop also contains the hypermutated N76, which is more likely to contact antigen. This position's hypermutation to proline suggests that it may alter the backbone conformation of its loop, although the mature murine homology model gives it phi/psi angles that work well for non-prolines as well.

### 1.4 Mutation in VH2, VH3

The mutation that was made in the above-listed VH regions is K75S. In this mutation, the large, charged K is removed but replaced with the common S rather than P, which is not seen in humans, for a much smaller risk of immunogenicity.

### 1.5 Mutation in VH3, VH4, VH5

The mutation that was made in the above listed VH regions is A23K, which is solvated and located around the corner from CDR-H1 and the loop containing hypermutated P75 and hypermutated N76. A23K could support N76, and the positively charged murine K here could also improve solubility. K and A are common in human (K 198/544, A 154/544 are the frequencies of these amino acids in a database of 544 human heavy chain sequences). This mutation was omitted from design VH2 so that design VH2 has a simple way to replace K75 but avoids the immunogenicity risk of both K75P and of D72V.

### 1.6 Mutations in VH3, VH5

The two mutations that were made in the above-listed VH regions are discussed below.

First, D72V is solvated and located around a corner from CDRs. However, this is in the framework loop with hypermutations P75 N76, which may therefore contact antigen. This position can interact with amino acid 75; positions 72 and 75 are on either side of a tight turn, so they must not be the same-charge or the turn could be destabilized. In mature murine, 72 and 75 are V and P; in acceptor, they are D and K. So if we there is a K75P mutation, D72V should be mutated as well. V at this position is uncommon in human (V 8/544, D 514/544, E 17/544 are the frequencies of these amino acids in a database of 544 human heavy chain sequences), so there is some immunogenicity risk with this mutation.

The second mutation, I69L is buried and supports CDR-H2. The I69Lmutation may help retain murine CDR structure, though no other residues nearby differ in mature murine vs. acceptor. L here is also common (I 122/544, L 342/544 are the frequencies of these amino acids in a database of 544 human heavy chain sequences), and is buried, so there is no immunogenicity risk with this mutation.

### 1.7 Mutation in VH2, VH4

The mutation that was made in the above-listed VH regions is G16E. This mutation has improved scFv domain stability in several other antibodies. The position is solvent-exposed, near a bend far from the CDRs. The E at this position is less common in human (E 44/544 vs. G 175/544 are the frequencies of these amino acids in a database of 544 human heavy chain sequences). This mutation was omitted from VH1 so that VH1 has only high priority changes; and was omitted from designs VH3 and VH5 to keep VH3 and VH5 the most murine-like designs.

### 1.8 Mutation in VH2, VH4, VH5

The mutation in the above-listed VH regions is E6Q which is mostly buried and located far from the CDRs. This mutation has improved scFv stability in several other antibodies. In this case, it is a mutation back to mature murine Q. Q at this position is common in human, though not so common in our acceptor framework's subgroup Heavy 3. This mutation was omitted from design VH1 so that VH1 has only high priority changes; and was omitted from design H3 to test the effect of this specific mutation.

### 1.9 Mutations in VH5

The two mutations that were made in VH5 are discussed below.

The first mutation, F67A is a Haidar position for CDR-H2 and is buried. The large F may clash with F63 on the grafted CDR, and therefore the F67A mutation is needed to retain mature CDR conformation. On the other hand, the acceptor framework's F is much larger than A to stick into this region buried between framework and CDR-H2, although the CDR graft model seems to fit well. The immunogenicity risk of this mutation is low because it is located at the far end of CDR-H2, which had no maturations from murine germline. F or A at this position are common in human (F 232/544, A 151/544), and the position is buried, so there is no immunogenicity risk. Leaving F here would mean that F63 and F67 are both F, which is unusual among human germlines, appearing in only the two subgroup 7 sequences.

The second mutation, V5Q is solvated and should not affect affinity. The V5Q mutation might improve solubility. V or Q at this position are common in human (V 212/544, vs. Q 182/544 are the frequencies of these amino acids in a database of 544 human heavy chain sequences).

### 2. Light Chain

### 2.1 Mutations in VL1, VL2, VL3, VL4

The three mutations that were made in the above-listed VL regions are discussed below.

The first mutation, V11L, is solvated on one side, between its beta strand and another (containing V19) which leads to CDR-L1. Because this residue is spatially close to residue 104, there is a chance that the slightly larger L11 and L104 (vs. V and V in acceptor) of the mature murine sequence could affect CDR-L1 structure. The acceptor framework V in this position is uncommon in human (V 4/496 vs. L 410/496, M 63/496 are the frequencies of these amino acids in a database of 496 human kappa chain sequences), therefore, mutation back to murine L is more likely to reduce immunogenicity than to increase it.

The second mutation, 121M is buried and is located on the strand which begins CDR-L1 three positions later. Therefore it could affect CDR-L1 structure. M or 1 in this position can almost contact L104 and L11. M in this position is common in human (M 91/496, vs. I 306/496, L 75/496 are the frequencies of these amino acids in a database of 496 human kappa chain sequences), and the position is buried so there is no immunogenicity risk.

The third mutation, V104L is buried, and located far from the CDRs. This position is packed next to V11, and there is a chance that the packing in this region could affect the CDRs. L in this position is most common in human (L 374/496 vs. V 121/496 are the frequencies of these amino acids in a database of 496 human kappa chain sequences).

### 2.2 Mutation in VL1, VL2, VL3

The mutation that was made in the above-listed VL regions is S60D, which is solvated. This position could possibly contact antigen and interacts with 54 in CDR-L2. In mature murine, 54 and this 60 are R and D; in acceptor, they are L and S. D at this position is also common in human (D 202/496, S 188/496 are the frequencies of these amino acids in a database of 496 human kappa chain sequences).

### 2.3 Mutations in VL2, VL3

The three mutations that were made in the above-listed VL regions are discussed below.

The first mutation, T22S is exposed to solvent and is located 2 positions before CDR-L1. All of its neighbors have the same sequence in mature murine as in acceptor. T22 of the acceptor hydrogen-bonds with the backbone oxygen of S7, the corresponding S of the mature marine's sequence should be able to do so as well, although it does not do so in the model. There is a small chance that this could affect CDR-L1 structure or antigen-binding. S at this position is common in human (S 257/496 vs. T 199/496 are the frequencies of these amino acids in a database of 496 human kappa chain sequences), so this mutation has no immunogenicity risk.

The second mutation, A43S is solvated on one side and located far from the CDRs. This position contacts the VH chain at VH G104. The VH region near this contact has the same sequence in mature murine and acceptor, so this difference should not affect pairing; but it is not far from CDR-H3, so there is a small chance that this contact could affect CDR-H3 structure. S at this position is common in human (S 268/496 vs. A 135/496 are the frequencies of these amino acids in a database of 496 human kappa chain sequences).

The third mutation, S63T, is exposed to solvent and is located next to CDR-L2, although it does not contact it. There is a chance that this could contact antigen. T at this position is also common in human (T 90/496 vs. S 389/496 are the frequencies of these amino acids in a database of 496 human kappa chain sequences).

### 2.4 Mutations in VL3

The four mutations that were made in the above-listed VL region are discussed below.

The first mutation, S12T is solvated and located far from the CDRs and antigen by distance and by chain distance. T at this framework position is a hypermutation from A in the murine germline, but it seems most likely to be a pointless hypermutation. T at this position is uncommon in human (T 8/496 vs. S 297/496, A 90/496, P 88/496 are the frequencies of these amino acids in a database of 496 human kappa chain sequences) (and for mouse), so there is some immunogenicity risk with this mutation.

The second mutation, Q100A points out into solvent and is not near the CDRs but not far down-chain from CDR-L3. In the structural models, Q here interacts with S9, or with the backbone of positions 5 and 6. This position could affect CDR-L3, but this is a small chance since the human Q in this position is mostly extended into solvent. A in this position is fairly common in human (A 64/496 vs. G 223/496, Q 143/496, S 58/496 are the frequencies of these amino acids in a database of 496 human kappa chain sequences).

The third mutation, A13V is solvated on one side and is located just before the beta turn at positions 13-18, one of whose strands leads to the distant CDR-L1. There is a chance that the mature murine V is important to CDR-L1 structure. V at this position is common in human (V 255/496, A 191/496 are the frequencies of these amino acids in a database of 496 human kappa chain sequences), so this mutation has very little immunogenicity risk.

The fourth mutation, L78V is buried far from the CDRs. V in this position is common in human (V 236/496 vs. L 219/496 are the frequencies of these amino acids in a database of 496 human kappa chain sequences), so this mutation has a very low immunogenicity risk.

### 2.5 Mutation in VL2

The mutation that was made in the VL2 region, DIN, is a Haidar position for CDR-L1 and is solvated. It could contact antigen and could also contact S93 S94 in CDR-L3 and Q56 in CDR-H2. This position could affect CDR-L3 when it changes length by −1 upon CDR grafting. N at this position from the mature murine is unusual for mouse (N 7/928 vs. D 598/928, E 135/928, Q 112/928), as it also is for human (6/496, vs. 379/496 D, 61/496 E are the frequencies of these amino acids in a database of 496 human kappa chain sequences), so this mutation has a small to medium relative risk of immunogenicity. This mutation was omitted from design VL3 to test the effect of the DIN mutation.

The sequences of the ALULA VH and VL regions, as well as the seven humanized ALULA variable heavy chain regions and five humanized ALULA variable light chain regions are shown below. CDRs 1, 2, and 3 are underlined in each amino acid sequence.

Variable Heavy Chain Sequences:
ALULA VH Variable Heavy Chain Amino Acid Sequence
(SEQ ID NO: 71)
EVQVQQSGTVLARPGASVKMSCKASGYTFTSYWMHWVKQRPGQGLEWIGAIYPGN
SDTSYNQKFKGKAKLTAVTSPNTAYMELSSLTNEDSAVYYCTTTTYGYDWFAYWG
QGTLVTVSA
hALULA VH0 Variable Heavy Chain Amino Acid Sequence
(SEQ ID NO: 1)
EVQLVESGGGLVKPGGSLRLSCAASGYTFTSYWMHWVRQAPGKGLEWVGAIYPGN
SDTSYNQKFKGRFTISRDDSKNTLYLQMNSLKTEDTAVYYCTTTTYGYDWFAYWG
QGTLVTVSS
hALULA VH0 Variable Heavy Chain Nucleic Acid Sequence
(SEQ ID NO: 95)
GAGGTGCAGC TTGTGGAGTC CGGCGGCGGC CTGGTGAAGC CCGGCGGCTC CCTGAGGCTG
TCCTGCGCCG CCTCCGGCTA CACCTTCACC TCCTACTGGA TGCACTGGGT GAGGCAGGCC
CCCGGCAAGG GCCTGGAGTG GGTGGGCGCC ATCTACCCCG GGAATTCTGA CACCTCCTAC
AACCAGAAGT TCAAGGGCAG GTTCACCATC TCCAGGGACG ACTCCAAGAA CACCCTGTAC
CTGCAGATGA ACTCCCTGAA GACCGAGGAC ACCGCCGTGT ACTACTGCAC CACCACCACC
TACGGCTACG ACTGGTTCGC CTACTGGGGC CAGGGCACCC TGGTGACCGT CTCGAGC
hALULA VH1 Variable Heavy Chain Amino Acid Sequence
(SEQ ID NO: 2)
EVQVVESGGGLVKPGGSLRLSCAASGYTFTSYWMHWVRQAPGKGLEWVGAIYPGN
SDTSYNQKFKGRFTISADTSKNTLYLQMNSLKTEDTAVYYCTTTTYGYDWFAYWG
QGTLVTVSS
hALULA VH1 Variable Heavy Chain Nucleic Acid Sequence
(SEQ ID NO: 96)
GAGGTGCAGG TGGTGGAGTC CGGCGGCGGC CTGGTGAAGC CCGGCGGCTC CCTGAGGCTG
TCCTGCGCCG CCTCCGGCTA CACCTTCACC TCCTACTGGA TGCACTGGGT GAGGCAGGCC
CCCGGCAAGG GCCTGGAGTG GGTGGGCGCC ATCTACCCCG GGAATTCTGA CACCTCCTAC
AACCAGAAGT TCAAGGGCAG GTTCACCATC TCCGCCGACA CCTCCAAGAA CACCCTGTAC
CTGCAGATGA ACTCCCTGAA GACCGAGGAC ACCGCCGTGT ACTACTGCAC CACCACCACC
TACGGCTACG ACTGGTTCGC CTACTGGGGC CAGGGCACCC TGGTGACCGT CTCGAGC
hALULA VH2 Variable Heavy Chain Amino Acid Sequence
(SEQ ID NO: 3)
EVQVVQSGGGLVKPGESLRLSCAASGYTFTSYWMHWVKQAPGKGLEWVGAIYPGN
SDTSYNQKFKGKFTISADTSSNTAYLQMNSLKTEDTAVYYCTTTTYGYDWFAYWG
QGTLVTVSS
hALULA VH2 Variable Heavy Chain Nucleic Acid Sequence
(SEQ ID NO: 97)
GAGGTGCAGG TGGTGCAGTC CGGCGGCGGC CTGGTGAAGC CCGGCGAGTC CCTGAGGCTG
TCCTGCGCCG CCTCCGGCTA CACCTTCACC TCCTACTGGA TGCACTGGGT GAAGCAGGCC
CCCGGCAAGG GCCTGGAGTG GGTGGGCGCC ATCTACCCCG GGAATTCTGA CACCTCCTAC
AACCAGAAGT TCAAGGGCAA GTTCACCATC TCCGCCGACA CCTCCTCCAA CACCGCCTAC
CTGCAGATGA ACTCCCTGAA GACCGAGGAC ACCGCCGTGT ACTACTGCAC CACCACCACC
TACGGCTACG ACTGGTTCGC CTACTGGGGC CAGGGCACCC TGGTGACCGT CTCGAGC
hALULA VH3 Variable Heavy Chain Amino Acid Sequence
(SEQ ID NO: 4)
EVQVVESGGGLVKPGGSLRLSCKASGYTFTSYWMHWVKQAPGKGLEWVGAIYPGN
SDTSYNQKFKGKFTLSAVTSSNTAYLQMNSLKTEDTAVYYCTTTTYGYDWFAYWG
QGTLVTVSS
hALULA VH3 Variable Heavy Chain Nucleic Acid Sequence
(SEQ ID NO: 98)
GAGGTGCAGG TGGTGGAGTC CGGCGGCGGC CTGGTGAAGC CCGGCGGCTC CCTGAGGCTG
TCCTGCAAGG CCTCCGGCTA CACCTTCACC TCCTACTGGA TGCACTGGGT GAAGCAGGCC
CCCGGCAAGG GCCTGGAGTG GGTGGGCGCC ATCTACCCCG GGAATTCTGA CACCTCCTAC
AACCAGAAGT TCAAGGGCAA GTTCACCCTG TCCGCCGTGA CCTCCTCCAA CACCGCCTAC
CTGCAGATGA ACTCCCTGAA GACCGAGGAC ACCGCCGTGT ACTACTGCAC CACCACCACC
TACGGCTACG ACTGGTTCGC CTACTGGGGC CAGGGCACCC TGGTGACCGT CTCGAGC
hALULA VH4 Variable Heavy Chain Amino Acid Sequence
(SEQ ID NO: 5)
EVQVVQSGGGLVKPGESLRLSCKASGYTFTSYWMHWVKQAPGKGLEWVGAIYPGN
SDTSYNQKFKGKFTISADTSPNTAYLQMNSLKTEDTAVYYCTTTTYGYDWFAYWG
QGTLVTVSS
hALULA VH4 Variable Heavy Chain Nucleic Acid Sequence
(SEQ ID NO: 99)
GAGGTGCAGG TGGTGCAGTC CGGCGGCGGC CTGGTGAAGC CCGGCGAGTC CCTGAGGCTG
TCCTGCAAGG CCTCCGGCTA CACCTTCACC TCCTACTGGA TGCACTGGGT GAAGCAGGCC
CCCGGCAAGG GCCTGGAGTG GGTGGGCGCC ATCTACCCCG GGAATTCTGA CACCTCCTAC
AACCAGAAGT TCAAGGGCAA GTTCACCATC TCCGCCGACA CCTCCCCCAA CACCGCCTAC
CTGCAGATGA ACTCCCTGAA GACCGAGGAC ACCGCCGTGT ACTACTGCAC CACCACCACC
TACGGCTACG ACTGGTTCGC CTACTGGGGC CAGGGCACCC TGGTGACCGT CTCGAGC
hALULA VH5 Variable Heavy Chain Amino Acid Sequence
(SEQ ID NO: 6)
EVQVQQSGGGLVKPGGSLRLSCKASGYTFTSYWMHWVKQAPGKGLEWVGAIYPG
NSDTSYNQKFKGKAT1SAVTSPNTAYLQMNSLKTEDTAVYYCTTTTYGYDWFAYW
GQGTLVTVSS
hALULA VH5 Variable Heavy Chain Nucleic Acid Sequence
(SEQ ID NO: 100)
GAGGTGCAGG TGCAGCAGTC CGGCGGCGGC CTGGTGAAGC CCGGCGGCTC CCTGAGGCTG
TCCTGCAAGG CCTCCGGCTA CACCTTCACC TCCTACTGGA TGCACTGGGT GAAGCAGGCC
CCCGGCAAGG GCCTGGAGTG GGTGGGCGCC ATCTACCCCG GGAATTCTGA CACCTCCTAC
AACCAGAAGT TCAAGGGCAA GGCCACCCTG TCCGCCGTGA CCTCCCCCAA CACCGCCTAC
CTGCAGATGA ACTCCCTGAA GACCGAGGAC ACCGCCGTGT ACTACTGCAC CACCACCACC
TACGGCTACG ACTGGTTCGC CTACTGGGGC CAGGGCACCC TGGTGACCGT CTCGAGC
hALULA VH6 Variable Heavy Chain Amino Acid Sequence
(SEQ ID NO: 7)
EVQVVESGGGLVKPGGSLRLSCAASGYTFTSYWMHWVRQAPGKGLEWVGRIKSKT
DGGTTDYAAPVKGRFTISADTSKNTLYLQMNSLKTEDTAVYYCTTTTYGYDWFAY
WGQGTLVTVSS
hALULA VH6 Variable Heavy Chain Nucleic Acid Sequence
(SEQ ID NO: 101)
GAGGTGCAGG TGGTGGAGTC CGGCGGCGGC CTGGTGAAGC CCGGCGGCTC CCTGAGGCTG
TCCTGCGCCG CCTCCGGCTA CACCTTCACC TCCTACTGGA TGCACTGGGT GAGGCAGGCC
CCCGGCAAGG GCCTGGAGTG GGTGGGCAGG ATCAAGTCCA AGACCGACGG CGGTACCACC
GACTACGCCG CCCCCGTGAA GGGCAGGTTC ACCATCTCCG CCGACACCTC CAAGAACACC
CTGTACCTGC AGATGAACTC CCTGAAGACC GAGGACACCG CCGTGTACTA CTGCACCACC
ACCACCTACG GCTACGACTG GTTCGCCTAC TGGGGCCAGG GCACCCTGGT GACCGTCTCG
AGC
Variable Light Chain Sequences:
ALULA VL Variable Light Chain Amino Acid Sequence
(SEQ ID NO: 72)
NIMMTQSPSSLTVSAGEKVTMSCKSSQSVLYSSNQKNYLAWYQQKPGQSPKLLIYW
ASTRESGVPDRFTGSGSGTDFTLTISSVQAEDLAVYYCHQYLSSLTFGAGTKLELK
hALULA VL0 Variable Light Chain Amino Acid Sequence
(SEQ ID NO: 8)
DIQMTQSPSSVSASVGDRVTITCKSSQSVLYSSNQKNYLAWYQQKPGKAPKLLIYWA
STRESGVPSRFSGSGSGTDFTLTISSLQPEDFATYYCHQYLSSLTFGQGTKVEIK
hALULA VL0 Variable Light Chain Nucleic Acid Sequence
(SEQ ID NO: 102)
GACATCCAGA TGACCCAGTC CCCCTCCTCC GTGTCCGCCT CCGTGGGCGA CAGGGTGACC
ATCACCTGCA AGTCCTCCCA GTCCGTGCTG TACAGCTCCA ACCAGAAGAA CTACCTGGCC
TGGTACCAGC AGAAGCCCGG CAAGGCCCCC AAGCTGCTGA TCTACTGGGC CTCCACCAGG
GAGTCCGGCG TGCCCTCCAG GTTCTCCGGC TCCGGCTCCG GCACCGACTT CACCCTGACC
ATCTCCTCCC TGCAGCCCGA GGACTTCGCC ACCTACTACT GCCACCAGTA CCTCTCGAGC
CTGACCTTCG GCCAGGGCAC CAAGGTGGAG ATCAAG
hALULA VL1 Variable Light Chain Amino Acid Sequence
(SEQ ID NO: 9)
DIQMTQSPSSLSASVGDRVTMTCKSSQSVLYSSNQKNYLAWYQQKPGKAPKLLIYW
ASTRESGVPDRFSGSGSGTDFTLTISSLQPEDFATYYCHQYLSSLTFGQGTKLEIK
hALULA VL1 Variable Light Chain Nucleic Acid Sequence
(SEQ ID NO: 34)
GACATCCAGA TGACCCAGTC CCCCTCCTCC CTGTCCGCCT CCGTGGGCGA CAGGGTGACC
ATGACCTGCA AGTCCTCCCA GTCCGTGCTG TACAGCTCCA ACCAGAAGAA CTACCTGGCC
TGGTACCAGC AGAAGCCCGG CAAGGCCCCC AAGCTGCTGA TCTACTGGGC CTCCACCAGG
GAGTCCGGCG TGCCCGACAG GTTCTCCGGC TCCGGCTCCG GCACCGACTT CACCCTGACC
ATCTCCTCCC TGCAGCCCGA GGACTTCGCC ACCTACTACT GCCACCAGTA CCTCTCGAGC
CTGACCTTCG GCCAGGGCAC CAAGCTGGAG ATCAAG
hALULA VL2 Variable Light Chain Amino Acid Sequence
(SEQ ID NO: 10)
NIQMTQSPSSLSASVGDRVTMSCKSSQSVLYSSNQKNYLAWYQQKPGKSPKLLIYW
ASTRESGVPDRFTGSGSGTDFTLTISSLQPEDFATYYCHQYLSSLTFGQGTKLEIK
hALULA VL2 Variable Light Chain Nucleic Acid Sequence
(SEQ ID NO: 53)
AACATCCAGA TGACCCAGTC CCCCTCCTCC CTGTCCGCCT CCGTGGGCGA CAGGGTGACC
ATGTCCTGCA AGTCCTCCCA GTCCGTGCTG TACAGCTCCA ACCAGAAGAA CTACCTGGCC
TGGTACCAGC AGAAGCCCGG CAAGTCCCCC AAGCTGCTGA TCTACTGGGC CTCCACCAGG
GAGTCCGGCG TGCCCGACAG GTTCACCGGC TCCGGCTCCG GCACCGACTT CACCCTGACC
ATCTCCTCCC TGCAGCCCGA GGACTTCGCC ACCTACTACT GCCACCAGTA CCTCTCGAGC
CTGACCTTCG GCCAGGGCAC CAAGCTGGAG ATCAAG
hALULA VL3 Variable Light Chain Amino Acid Sequence
(SEQ ID NO: 11)
DIQMTQSPSSLTVSVGDRVTMSCKSSQSVLYSSNQKNYLAWYQQKPGKSPKLLIYW
ASTRESGVPDRFTGSGSGTDFTLTISSVQPEDFATYYCHQYLSSLTFGAGTKLEIK
hALULA VL3 Variable Light Chain Nucleic Acid Sequence
(SEQ ID NO: 54)
GACATCCAGA TGACCCAGTC CCCCTCCTCC CTGACCGTGT CCGTGGGCGA CAGGGTGACC
ATGTCCTGCA AGTCCTCCCA GTCCGTGCTG TACAGCTCCA ACCAGAAGAA CTACCTGGCC
TGGTACCAGC AGAAGCCCGG CAAGTCCCCC AAGCTGCTGA TCTACTGGGC CTCCACCAGG
GAGTCCGGCG TGCCCGACAG GTTCACCGGC TCCGGCTCCG GCACCGACTT CACCCTGACC
ATCTCCTCCG TGCAGCCCGA GGACTTCGCC ACCTACTACT GCCACCAGTA CCTCTCGAGC
CTGACCTTCG GCGCCGGCAC CAAGCTGGAG ATCAAG
hALULA VL4 Variable Light Chain Amino Acid Sequence
(SEQ ID NO: 12)
DIQMTQSPSSLSASVGDRVTMTCKSSQSVLYSSNQKNYLAWYQQKPGKAPKLLIYA
ASSLQSGVPSRFSGSGSGTDFTLTISSLQPEDFATYYCHQYLSSLTFGQGTKLEIK
hALULA VL4 Variable Light Chain Nucleic Acid Sequence
(SEQ ID NO: 55)
GACATCCAGA TGACCCAGTC CCCCTCCTCC CTGTCCGCCT CCGTGGGCGA CAGGGTGACC
ATGACCTGCA AGTCCTCCCA GTCCGTGCTG TACAGCTCCA ACCAGAAGAA CTACCTGGCC
TGGTACCAGC AGAAGCCCGG CAAGGCCCCC AAGCTGCTGA TCTACGCCGC CTCCTCCCTG
CAGTCCGGCG TGCCCTCCAG GTTCTCCGGC TCCGGCTCCG GCACCGACTT CACCCTGACC
ATCTCCTCCC TGCAGCCCGA GGACTTCGCC ACCTACTACT GCCACCAGTA CCTCTCGAGC
CTGACCTTCG GCCAGGGCAC CAAGCTGGAG ATCAAG

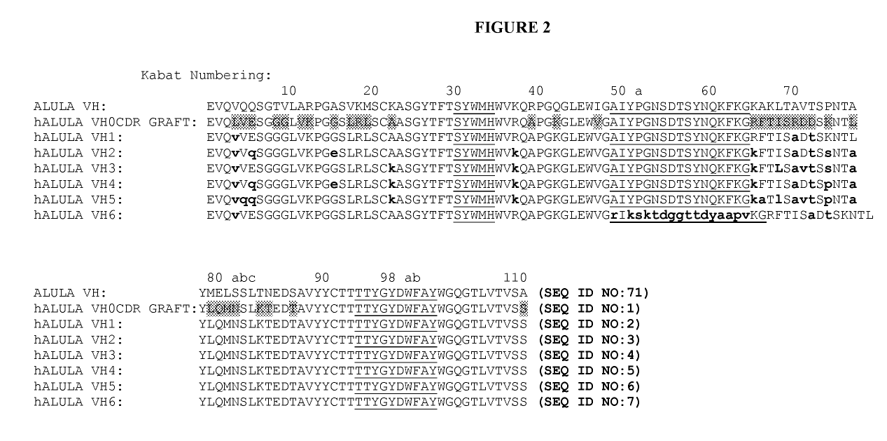

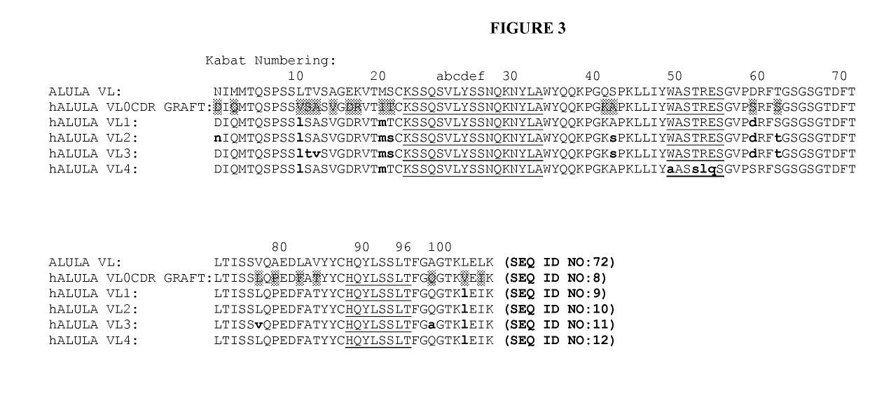

Alignments of the amino acid sequences of ALULA VH and VL with the seven humanized ALULA variable heavy chain regions and five humanized ALULA variable light chain regions are shown in FIGS. 2 and 3, respectively.

### Example 3: Materials & Methods

Production of human soluble αvβ5 protein: The extracellular domains of human αv and β5 integrin subunits were cloned into mammalian expression vectors and stably transfected in CHO cells. Protein was expressed using standard methods and purified from the conditioned media using affinity chromatography on an immobilized αv integrin-specific monoclonal antibody.

Solid-Phase αvβ5 Binding Assay (ELISA): A 96-well microtiter plate was precoated with streptavidin (Thermo Scientific Reacti-Bind StreptAvidin Coated High Binding Capacity plate) was used. Biotinylated soluble αvβ5 protein (2 μg/mL) in TBS with 1% BSA was added to the wells, and the plate was incubated for 1 hr at 25° C. The plate was washed with wash buffer (0.05% Tween-20 in PBS), and purified humanized ALULA antibody in TBS containing 1% BSA, 1 mM CaCl2, and 1 mM MgCl2 were added (50 μl/well). The plate was incubated for 1 hr at 25° C., washed, and then incubated for 1 hr with 50 μl/well of peroxide-conjugated goat anti-human secondary antibody. Bound antibody was detected using 3,3′,5,5′-tetramethylbenzidine (TMB). Binding was indicated by the absorbency measured at 450 nm.

Competition ELISA: A 96-well microtiter plate was coated with 50 μl/well of 5 μg/mL soluble human αvβ5 protein at 4° C. overnight. The plate was washed with wash buffer (0.05% Tween-20 in PBS) four times in an automated plate washer. 300 μl/well of 1% BSA in PBS was added and incubated for 1 hr at 25° C. to block nonspecific binding. The plate was washed as above, and dilutions of humanized antibodies mixed with 1 nM murine ALULA in PBS containing 1% BSA, were added (50 μl/well). The plate was incubated for 1 hr at 25° C., washed, and then incubated for 40 minutes with 100 μl/well of peroxide-conjugated goat anti-mouse antibody. Bound antibody was detected using 3,3′,5,5′-tetramethylbenzidine (TMB). Binding was indicated by the absorbency measured at 450 nm.

Vitronectin Inhibition ELISA: 96-well microtiter plates were coated with 5 μg/ml purified human plasma vitronectin diluted in PBS (50 μl/well) at 4° C. overnight. After the coating solution was removed, the plates were blocked with 300 μl/well of 1% BSA/TBS at 25° C. for 1 hr. The plate was washed with wash buffer (0.05% Tween-20 in TBS containing 1 mM CaCl₂ and 1 mM MgCl₂), and dilutions of humanized antibodies mixed with 1 nM soluble αvβ5 protein in TBS containing 1% BSA, 1 mM CaCl₂ and 1 mM MgCl₂ were added (50 μl/well) and incubated at 25° C. for 1 hr. The plate was washed 4 times with wash buffer in an automated plate washer and incubated sequentially with 50 μl/well of the anti-beta5 monoclonal antibody 15F11 (at 0.5 ug/mL) in for 1 hr at 25° C. in TBS containing 1% BSA, 1 mM CaCl₂ and 1 mM MgCl₂. After washing the plate 4 times with wash buffer, 100 μl/well of a 1:5000 dilution of a peroxidase-conjugated goat anti-mouse antibody in TBS containing 1% BSA, 1 mM CaCl₂ and 1 mM MgCl₂ were added and incubated for 1 hr at 25° C. Bound protein was detected using the TMB substrate and indicated by the absorbency measured at 450 nm.

Vitronectin Adhesion Assay: A 96-well microtiter plate was coated with 50 μl/well of 10 μg/ml purified human vitronectin diluted in phosphate buffered saline (PBS) at 4° C. overnight. The plate was washed twice with PBS (100 μl/well) and blocked with 1% BSA in PBS (100 μl/well) for 1 hr at 25° C. The plate was washed twice with 100 μl/well of assay buffer (TBS complete plus 1 mM CaCl₂ and 1 mM MgCl₂). Next, to the individual wells of the plate were added 25 μl of a hybridoma supernatant (or a purified antibody) and 25 μl of αvβ5-BaF3 cells (5×10⁶ cells/ml, labeled with 2 μM Calcein AM). The plate was incubated at 37° C. for 1.5 hr, and then washed 4-6 times with the assay buffer (100 μl/well). The fluorescence emitted from cells captured on the plate was recorded. Percentage binding was determined by comparing the pre-washed fluorescence signal (i.e., total cells added) to that after washing (i.e., bound cells).

FACS Binding Assay: Cells were washed one time in PBS, and then resuspended in FACS buffer (1×PBS, 1% BSA, 1 mM CaCl₂, and 1 mM MgCl₂). 1×10⁶ cells were then incubated on ice for 1 hr in FACS buffer containing the test antibody in a total volume of 50 μl. After incubation, the cells were washed two times with ice cold FACS buffer, resuspended in 50 μl of FACS buffer containing 3 μg/ml goat anti-mouse IgG AlexaFluor488 (Jackson ImmunoResearch), and incubated on ice for 30 min. The cells were then washed twice with ice cold FACS buffer and fixed in 1% PFA overnight. Cell aggregates were removed by filtering through 40 μm filter plates (Millipore) and binding of the labeled secondary antibody was monitored by flow cytometry using a FACS Calibur (BD Biosciences).

Unilateral Ischemic Clamp Model: Male Sprague—Dawley rats weighing 250-320 g were purchased from Harlan Laboratories and allowed to acclimate for 5 days with ad libitum food and water prior to surgery. The animals were anesthetized with an Isofluorane/O2 mixture, 5% for induction and 1-2% for maintenance of anesthesia. The abdomen was opened using a 3 cm midline incision. Each kidney was isolated and the fat and connective tissue surrounding the renal artery and vein were dissected away using sterile cotton swabs. The right kidney was removed and the renal artery and vein sutured off. Ischemia of the left kidney was initiated by clamping the renal artery and vein for 40 minutes using non-traumatic clamps on the renal pedicle. At the conclusion of the ischemic period, the clamp was removed and the kidney was observed to insure rapid re-establishment of blood flow. The studies were terminated at 72 hours post-surgery and the rats were euthanized by pentobarbital overdose followed by cervical dislocation. Test agents (control antibody, 1E6,—a mIgG1 isotype control) or H4/L2 anti-αvβ5 antibody) were administered by subcutaneous injection in a 300 μL volume 6 hours before clamping. A 0.15 mL venous blood sample was drawn at study initiation for baseline creatinine measurement and at 48 hrs post-surgery for pathological serum creatinine level evaluation. Creatinine concentration was measured on a Beckman Creatinine Analyzer 2. The machine was standardized with a known control and the samples were run using a picric acid reaction.

### Example 4: Screening of Humanized ALULA Constructs

Screening of humanized constructs was carried out using proteins expressed transiently in CHO cells corresponding to the variable domain heavy and variable light chain designs, with the VH domain fused to an aglycosylated human IgG1 domain (containing a Thr299Ala mutation, numbered according to the Kabat numbering convention) and the VL domain fused to a kappa light chain.

Screening of all combinations of heavy chain versions VH0, VH1, VH2, VH3, VH4, or VH5, with light chains VL0, VL1, VL2, or VL3 was carried out using unpurified proteins in conditioned cell media. The binding of each construct to biotin-conjugated human αvβ5 protein was determined using a solid-phase binding assay (ELISA) and by FACS, and the ability of each construct to block ligand binding was determined using a vitronectin cell adhesion assay (Table 1).

TABLE 1
Summary of Binding and Inhibition Data
with Unpurified Agly-IgG1 constructs

| | | FACS, | |
|---|---|---|---|
| | | αvβ5- | VN adhesion |
| | ELISA, | BaF3 | inhibition, |
| | biotin-αvβ5 | (EC50, | αvβ5-BaF3 |
| Humanized ALULA Antibody | (EC50, nM) | nM) | (IC50, nM) |
| VH0/VL0 Agly-hIgG1(T299A) | 1.2 | 1122.5 | 1769.2 |
| VH0/VL1 Agly-hIgG1(T299A) | 1.3 | 798.3 | 1620.6 |
| VH0/VL2 Agly-hIgG1(T299A) | 1.0 | 855.2 | 1969.9 |
| VH0/VL3 Agly-hIgG1(T299A) | 0.9 | 1274.3 | 494.3 |
| VH1/VL0 Agly-hIgG1(T299A) | 1.0 | 3677.4 | 3295.2 |
| VH1/VL1 Agly-hIgG1(T299A) | 1.9 | 3356.8 | 2523.6 |
| VH1/VL2 Agly-hIgG1(T299A) | 2.3 | 5373.1 | 1728.1 |
| VH1/VL3 Agly-hIgG1(T299A) | 2.9 | 2925.8 | 1841.1 |
| VH2/VL0 Agly-hIgG1(T299A) | 0.5 | 354.5 | 475.7 |
| VH2/VL1 Agly-hIgG1(T299A) | 1.0 | 404.6 | 317.6 |
| VH2/VL2 Agly-hIgG1(T299A) | 0.7 | 645.8 | 291.6 |
| VH2/VL3 Agly-hIgG1(T299A) | 1.0 | 989.6 | 303.0 |
| VH3/VL0 Agly-hIgG1(T299A) | ND* | ND | ND |
| VH3/VL1 Agly-hIgG1(T299A) | 1.2 | 1926.0 | 360.3 |
| VH3/VL2 Agly-hIgG1(T299A) | 0.7 | 3310.0 | 124.5 |
| VH3/VL3 Agly-hIgG1(T299A) | 1.2 | 6287.5 | 227.4 |
| VH4/VL0 Agly-hIgG1(T299A) | 0.3 | 158.5 | 74.9 |
| VH4/VL1 Agly-hIgG1(T299A) | 0.4 | 125.5 | 59.8 |
| VH4/VL2 Agly-hIgG1(T299A) | 0.4 | 155.9 | 71.8 |
| VH4/VL3 Agly-hIgG1(T299A) | 1.7 | 162.5 | 64.8 |
| VH5/VL0 Agly-hIgG1(T299A) | 0.4 | 243.7 | 217.7 |
| VH5/VL1 Agly-hIgG1(T299A) | 0.3 | 147.2 | 162.9 |
| VH5/VL2 Agly-hIgG1(T299A) | 0.4 | 358.7 | 83.2 |
| VH5/VL3 Agly-hIgG1(T299A) | 1.4 | 421.9 | 72.2 |

*ND, Not determined due to low expression level

A subset of the heavy-light chain pairs were purified and tested further.

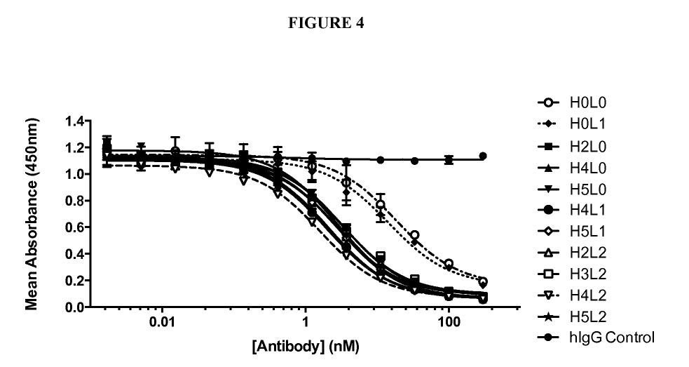

The binding of each construct to soluble, purified human αvβ5 protein was determined by a competition ELISA with murine ALULA (FIG. 4). The IC50 values determined in this assay are shown in Table 2 below. Constructs containing H0 had notably diminished abilities to block ALULA binding, indicating that the amino acid changes in H0 chain compared to the ALULA framework reduced the binding affinity of the H0 antibody. The remaining constructs behaved similarly, with version H4/L2 showing the lowest IC50 value in this assay.

In parallel, the humanized ALULA antibodies were tested in two adhesion assays. One measured the ability of each antibody to inhibit the binding of soluble αvβ5 to purified human plasma vitronectin. In the other assay, the murine BaF3 cell line was stably transfected with human αv and β5 subunits, and the adhesion of these cells to vitronectin was determined using a fluorescence endpoint. The results of both adhesion assays paralleled the binding assay, in that the H0-containing constructs were significantly less effective inhibitors, while H4/L2 was among the most effective (see, Table 2).

TABLE 2
Summary of Binding and Inhibition Data with Purified Agly-IgG1 Proteins

| | | VN | |
|---|---|---|---|
| | | inhibition | |
| | | ELISA, | |
| | Competition | soluble | VN adhesion |
| | ELISA, | αvβ5 | inhibition, |
| | soluble αvβ5 | (IC50, | αvβ5-BaF3 |
| Humanized ALULA Antibody | (IC50, nM) | nM) | (IC50, nM) |
| VH4/VL2 Agly-hIgG1(T299A) | 1.6 | 0.6 | 0.5 |
| VH5/VL2 Agly-hIgG1(T299A) | 2.0 | 1.3 | 0.9 |
| VH4/VL0 Agly-hIgG1(T299A) | 2.0 | 1.7 | 1.1 |
| VH4/VL1 Agly-hIgG1(T299A) | 2.2 | 0.9 | 1.0 |
| VH5/VL0 Agly-hIgG1(T299A) | 2.8 | 3.1 | 1.0 |
| VH5/VL1 Agly-hIgG1(T299A) | 3.0 | 1.3 | 0.7 |
| VH2/VL2 Agly-hIgG1(T299A) | 2.8 | 1.1 | 0.4 |
| VH3/VL2 Agly-hIgG1(T299A) | 3.3 | 2.5 | 0.6 |
| VH2/VL0 Agly-hIgG1(T299A) | 3.4 | 2.4 | 1.3 |
| VH0/VL1 Agly-hIgG1(T299A) | 14.2 | 11.9 | 2.2 |
| VH0/VL0 Agly-hIgG1(T299A) | 18.3 | 11.1 | 3.0 |

### Example 5: Selection of Human Constant Domain for Humanized ALULA

In order to minimize the immune effector function of the humanized ALULA constructs, an engineered IgG Fc domain was selected that has been shown to bind with low affinity to Fcγ receptors and complement C1q (see, US Patent Publication No. 2012/0100140A1). The heavy chain (referred to as “IgG4.P (S228P)/IgG1(N297Q)” comprises the CH1 and CH2 domains of human IgG4, and the CH3 domain of human IgG1. An amino acid change (S228P using the Kabat numbering convention) in the hinge region stabilizes the heavy chain inter-chain disulfide bond to minimize antibody rearrangement, and the N297Q mutation eliminates the N-glycosylation site. The light chain contains a human kappa CL domain.

### Example 6: Screening of ALULA Constructs

Based on the results described above in Example 4, four humanized ALULA constructs: VH2/VL2, VH4/VL0, VH4/VL2, and VH5/VL2, were selected for further testing. These VH/VL constructs were expressed as fusion proteins with the hybrid, aglycosylated IgG4.P (S228P)/IgG1 (N297Q) domain, and human kappa light chain domain, and purified from the conditioned medium of stably transfected CHO cells. Each humanized ALULA construct was assessed for binding using competition ELISA with murine ALULA and by FACS using human αvβ5-transfected BaF3 cells. Each humanized ALULA construct was also assessed for blocking binding/adhesion to vitronectin by ELISA or cell adhesion assays using BaF3 cells stably transfected with human or cynomolgous monkey αvβ5. The humanized ALULA constructs were compared to a chimeric form of ALULA comprising the murine heavy and light chain variable domains fused to human IgG1 heavy chain and human kappa light chain domains. The four humanized ALULA constructs behaved similarly in the assays, with H4/L2 generally having similar or slightly higher affinity/blocking potency than the other constructs (see, Table 3).

TABLE 3
Summary of Binding and Inhibition Data with Purified
Agly-IgG4P/IgG1 Proteins

| | | | | | | | BaF3-cyno | | | |
|---|---|---|---|---|---|---|---|---|---|---|
| | ALULA | | αvβ5- | | BaF3-hu αvβ5 | | αvβ5 | | | |
| | Competition | | vitronectin | | Cell adhesion to | | Cell adhesion to | | FACS | |
| | ELISA | | Blocking | | vitronectin | | vitronectin | | BaF3-hu | |
| | (IC₅₀, nM) | | ELISA (IC₅₀, nM) | | (IC₅₀, nM) | | (IC₅₀, nM) | | αvβ5 (EC₅₀, nM) | |
| Construct | IC₅₀ | Fold | IC₅₀ | Fold | IC₅₀ | Fold | IC₅₀ | Fold | EC₅₀ | Fold |
| (Agly-IgG4P/G1) | (nM) | chg. | (nM) | chg. | (nM) | chg. | (nM) | chg. | (nM) | chg. |
| VH4/VL2 | 1.84 | 1.0 | 1.06 | 0.7 | 0.10 | 0.6 | 0.32 | 0.5 | 2.92 | 0.6 |
| VH4/VL0 | 1.84 | 1.0 | 1.52 | 1.0 | 0.09 | 0.5 | 0.43 | 0.7 | 2.52 | 0.5 |
| VH5/VL2 | 1.85 | 1.0 | 1.24 | 0.8 | 0.29 | 1.7 | 0.63 | 1.0 | 2.90 | 0.5 |
| VH2/VL2 | 2.61 | 1.4 | 1.74 | 1.1 | 0.30 | 1.8 | 0.61 | 1.0 | 5.73 | 1.1 |
| Mouse | 1.81 | — | 1.60 | — | 0.17 | — | 0.61 | — | 5.06 | — |
| chimeric | | | | | | | | | | |
| ALULA | | | | | | | | | | |

Three additional combinations (VH4/VL4, VH6/VL2, and VH6/VL4) were produced on the IgG4.P (S228P)/IgG1 (N297Q) backbone to determine the effect of the changes to CDRH2 utilized in designs VH6 and VL4. Each of these 3 constructs blocked murine ALULA and inhibited αvβ5-vitronectin binding with IC50 values≧100 nM, indicating that the changes to CDRH2 incorporated into designs VH6 and VL4 significantly impaired binding of the antibody to αvβ5 (data not shown).

### Example 7: ELISA Binding Data with Fabs of Selected Humanized ALULA Constructs

Fab fragments of the four constructs of Example 6 were generated from the corresponding IgG1(N297Q) versions by digestion with papain. The ability of each Fab to block 2 nM soluble αvβ5 binding to immobilized human plasma vitronectin was determined by ELISA. As shown in Table 4, the VH4/VL2 Fab fragment blocked ligand binding with comparable IC50 to the chimeric murine antibody, while the other versions had slightly higher IC50 values.

TABLE 4
Inhibition of αvβ5 binding to Vitronectin Using Fab fragments

αvβ5-vitronectin
Blocking ELISA
(IC₅₀ nM)

| Fab Source | IC₅₀ (nM) | Fold change |
|---|---|---|
| H4/L2 | 4.3 | 0.9 |
| H4/L0 | 7.5 | 1.6 |
| H5/L2 | 6.8 | 1.4 |
| H2/L2 | 7.3 | 1.5 |
| Mouse chimeric ALULA | 4.8 | — |

### Example 8: Exemplary Humanized Anti-αvβ5 Antibodies

Four exemplary humanized anti-αvβ5 antibodies are provided below. These include designs based on VH domains VH4, VH5, and VH2 and VL domains VL0 and VL2.

The heavy chain of these exemplary antibodies comprises the VH4, VH2, or VH5 variable heavy chain (see, FIG. 2) and a constant region comprising the CH1 and CH2 domains of human IgG4P and the CH3 domain of human IgG1. The heavy chain further includes a mutation in the hinge region (S228P, Kabat numbering) to reduce the formation of half antibodies and a mutation in the CH2 domain to eliminate an N-glycosylation site (N297Q, Kabat numbering). In each sequence, VHCDR1, VHCDR2, and VHCDR3 are underlined; the IgG4P constant domain, CH1 and CH2 domains are in bold; the IgG1 constant domain and CH3 domain are italicized; and both the S228P mutation in the IgG4P hinge and the N297Q mutation in the CH2 domain are in bold, underlined font.

Heavy Chain Based on VH4:
(SEQ ID NO: 69)
EVQVVQSGGGLVKPGESLRLSCKASGYTFTSYWMHWVKQAPGKGLEWVGA
IYPGNSDTSYNQKFKGKFTISADTSPNTAYLQMNSLKTEDTAVYYCTTTT
YGYDWFAYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFQSTYR
VVSVLTVLHQDWLNGKEYKCKVSNKGLPSSIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG
Heavy Chain Based on VH2:
(SEQ ID NO: 80)
EVQVVQSGGGLVKPGESLRLSCAASGYTFTSYWMHWVKQAPGKGLEWVGA
IYPGNSDTSYNQKFKGKFTISADTSSNTAYLQMNSLKTEDTAVYYCTTTT
YGYDWFAYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFQSTYR
VVSVLTVLHQDWLNGKEYKCKVSNKGLPSSIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG
Heavy Chain Based on VH5:
(SEQ ID NO: 81)
EVQVQQSGGGLVKPGGSLRLSCKASGYTFTSYWMHWVKQAPGKGLEWVGA
IYPGNSDTSYNQKFKGKATLSAVTSPNTAYLQMNSLKTEDTAVYYCTTTT
YGYDWFAYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFQSTYR
VVSVLTVLHQDWLNGKEYKCKVSNKGLPSSIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG

The exemplary light chains comprise the VL0 or VL2 variable light chain (see FIG. 3) and a constant region comprising the kappa light chain constant region of human IgG4P (italicized below). The CDRs based on Kabat are underlined.

Light Chain Based on VL0:
(SEQ ID NO: 82)
DIQMTQSPSSVSASVGDRVTITCKSSQSVLYSSNQKNYLAWYQQKPGKAP
KLLIYWASTRESGVPSRFSGSGSGTDFTLTISSLQPEDFATYYCHQYLSS
LTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Light Chain Based on VL2:
(SEQ ID NO: 70)
NIQMTQSPSSLSASVGDRVTMSCKSSQSVLYSSNQKNYLAWYQQKPGKSP
KLLIYWASTRESGVPDRFTGSGSGTDFTLTISSLQPEDFATYYCHQYLSS
LTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

### Example 9: Exemplary Nucleic Acid Sequences of Anti-αvβ5 Antibodies

The following are exemplary nucleic acid sequences encoding heavy chains based on the humanized ALULA VH0-VH6 designs described above. The heavy chains of these exemplary antibodies have a constant region comprising the CH1 and CH2 domains of human IgG4P and the CH3 domain of human IgG1. The heavy chain further includes a mutation in the hinge region (S228P, Kabat numbering) to reduce the formation of half antibodies and a mutation in the CH2 domain to eliminate an N-glycosylation site (N297Q, Kabat numbering).

| Nucleic Acid Sequence of Heavy Chain Based on VH0: | |
|---|---|
| (SEQ ID NO: 83) | |
| 1 | GAGGTGCAGC TTGTGGAGTC CGGCGGCGGC CTGGTGAAGC CCGGCGGCTC CCTGAGGCTG |
| 61 | TCCTGCGCCG CCTCCGGCTA CACCTTCACC TCCTACTGGA TGCACTGGGT GAGGCAGGCC |
| 121 | CCCGGCAAGG GCCTGGAGTG GGTGGGCGCC ATCTACCCCG GGAATTCTGA CACCTCCTAC |
| 181 | AACCAGAAGT TCAAGGGCAG GTTCACCATC TCCAGGGACG ACTCCAAGAA CACCCTGTAC |
| 241 | CTGCAGATGA ACTCCCTGAA GACCGAGGAC ACCGCCGTGT ACTACTGCAC CACCACCACC |
| 301 | TACGGCTACG ACTGGTTCGC CTACTGGGGC CAGGGCACCC TGGTGACCGT CTCGAGCGCC |
| 361 | TCCACCAAGG GCCCATCCGT CTTCCCCCTG GCGCCCTGCT CCAGATCTAC CTCCGAGAGC |
| 421 | ACAGCCGCCC TGGGCTGCCT GGTCAAGGAC TACTTCCCCG AACCGGTGAC GGTGTCGTGG |
| 481 | AACTCAGGCG CCCTGACCAG CGGCGTGCAC ACCTTCCCGG CTGTCCTACA GTCCTCAGGA |
| 541 | CTCTACTCCC TCAGCAGCGT GGTGACCGTG CCCTCCAGCA GCTTGGGCAC GAAGACCTAC |
| 601 | ACCTGCAACG TAGATCACAA GCCCAGCAAC ACCAAGGTGG ACAAGAGAGT TGAGTCCAAA |
| 661 | TATGGTCCCC CATGCCCACC GTGCCCAGCA CCTGAGTTCC TGGGGGGACC ATCAGTCTTC |
| 721 | CTGTTCCCCC CAAAACCCAA GGACACTCTC ATGATCTCCC GGACCCCTGA GGTCACGTGC |
| 781 | GTGGTGGTGG ACGTGAGCCA GGAAGACCCC GAGGTCCAGT TCAACTGGTA CGTGGATGGC |
| 841 | GTGGAGGTGC ATAATGCCAA GACAAAGCCG CGGGAAGAGC AGTTCCAGAG CACGTACCGT |
| 901 | GTGGTCAGCG TCCTCACCGT CCTGCACCAG GACTGGCTGA ACGGCAAGGA GTACAAGTGC |
| 961 | AAGGTCTCCA ACAAAGGCCT CCCGTCCTCC ATCGAGAAAA CCATCTCCAA AGCCAAAGGG |
| 1021 | CAGCCCCGAG AGCCACAAGT GTACACCCTG CCCCCATCCC GGGATGAGCT GACCAAGAAC |
| 1081 | CAGGTCAGCC TGACCTGCCT GGTCAAAGGC TTCTATCCCA GCGACATCGC CGTGGAGTGG |
| 1141 | GAGAGCAATG GGCAGCCGGA GAACAACTAC AAGACCACGC CTCCCGTGTT GGACTCCGAC |
| 1201 | GGCTCCTTCT TCCTCTACAG CAAGCTCACC GTGGACAAGA GCAGGTGGCA GCAGGGGAAC |
| 1261 | GTCTTCTCAT GCTCCGTGAT GCATGAGGCT CTGCACAACC ACTACACGCA GAAGAGCCTC |
| 1321 | TCCCTGTCTC CCGGTTGA |
| Nucleic Acid Sequence of Heavy Chain Based on VH1: | |
| (SEQ ID NO: 84) | |
| 1 | GAGGTGCAGG TGGTGGAGTC CGGCGGCGGC CTGGTGAAGC CCGGCGGCTC CCTGAGGCTG |
| 61 | TCCTGCGCCG CCTCCGGCTA CACCTTCACC TCCTACTGGA TGCACTGGGT GAGGCAGGCC |
| 121 | CCCGGCAAGG GCCTGGAGTG GGTGGGCGCC ATCTACCCCG GGAATTCTGA CACCTCCTAC |
| 181 | AACCAGAAGT TCAAGGGCAG GTTCACCATC TCCGCCGACA CCTCCAAGAA CACCCTGTAC |
| 241 | CTGCAGATGA ACTCCCTGAA GACCGAGGAC ACCGCCGTGT ACTACTGCAC CACCACCACC |
| 301 | TACGGCTACG ACTGGTTCGC CTACTGGGGC CAGGGCACCC TGGTGACCGT CTCGAGCGCC |
| 361 | TCCACCAAGG GCCCATCCGT CTTCCCCCTG GCGCCCTGCT CCAGATCTAC CTCCGAGAGC |
| 421 | ACAGCCGCCC TGGGCTGCCT GGTCAAGGAC TACTTCCCCG AACCGGTGAC GGTGTCGTGG |
| 481 | AACTCAGGCG CCCTGACCAG CGGCGTGCAC ACCTTCCCGG CTGTCCTACA GTCCTCAGGA |
| 541 | CTCTACTCCC TCAGCAGCGT GGTGACCGTG CCCTCCAGCA GCTTGGGCAC GAAGACCTAC |
| 601 | ACCTGCAACG TAGATCACAA GCCCAGCAAC ACCAAGGTGG ACAAGAGAGT TGAGTCCAAA |
| 661 | TATGGTCCCC CATGCCCACC GTGCCCAGCA CCTGAGTTCC TGGGGGGACC ATCAGTCTTC |
| 721 | CTGTTCCCCC CAAAACCCAA GGACACTCTC ATGATCTCCC GGACCCCTGA GGTCACGTGC |
| 781 | GTGGTGGTGG ACGTGAGCCA GGAAGACCCC GAGGTCCAGT TCAACTGGTA CGTGGATGGC |
| 841 | GTGGAGGTGC ATAATGCCAA GACAAAGCCG CGGGAAGAGC AGTTCCAGAG CACGTACCGT |
| 901 | GTGGTCAGCG TCCTCACCGT CCTGCACCAG GACTGGCTGA ACGGCAAGGA GTACAAGTGC |
| 961 | AAGGTCTCCA ACAAAGGCCT CCCGTCCTCC ATCGAGAAAA CCATCTCCAA AGCCAAAGGG |
| 1021 | CAGCCCCGAG AGCCACAAGT GTACACCCTG CCCCCATCCC GGGATGAGCT GACCAAGAAC |
| 1081 | CAGGTCAGCC TGACCTGCCT GGTCAAAGGC TTCTATCCCA GCGACATCGC CGTGGAGTGG |
| 1141 | GAGAGCAATG GGCAGCCGGA GAACAACTAC AAGACCACGC CTCCCGTGTT GGACTCCGAC |
| 1201 | GGCTCCTTCT TCCTCTACAG CAAGCTCACC GTGGACAAGA GCAGGTGGCA GCAGGGGAAC |
| 1261 | GTCTTCTCAT GCTCCGTGAT GCATGAGGCT CTGCACAACC ACTACACGCA GAAGAGCCTC |
| 1321 | TCCCTGTCTC CCGGTTGA |
| Nucleic Acid Sequence of Heavy Chain Based on VH2: | |
| (SEQ ID NO: 85) | |
| 1 | GAGGTGCAGG TGGTGCAGTC CGGCGGCGGC CTGGTGAAGC CCGGCGAGTC CCTGAGGCTG |
| 61 | TCCTGCGCCG CCTCCGGCTA CACCTTCACC TCCTACTGGA TGCACTGGGT GAAGCAGGCC |
| 121 | CCCGGCAAGG GCCTGGAGTG GGTGGGCGCC ATCTACCCCG GGAATTCTGA CACCTCCTAC |
| 181 | AACCAGAAGT TCAAGGGCAA GTTCACCATC TCCGCCGACA CCTCCTCCAA CACCGCCTAC |
| 241 | CTGCAGATGA ACTCCCTGAA GACCGAGGAC ACCGCCGTGT ACTACTGCAC CACCACCACC |
| 301 | TACGGCTACG ACTGGTTCGC CTACTGGGGC CAGGGCACCC TGGTGACCGT CTCGAGCGCC |
| 361 | TCCACCAAGG GCCCATCCGT CTTCCCCCTG GCGCCCTGCT CCAGATCTAC CTCCGAGAGC |
| 421 | ACAGCCGCCC TGGGCTGCCT GGTCAAGGAC TACTTCCCCG AACCGGTGAC GGTGTCGTGG |
| 481 | AACTCAGGCG CCCTGACCAG CGGCGTGCAC ACCTTCCCGG CTGTCCTACA GTCCTCAGGA |
| 541 | CTCTACTCCC TCAGCAGCGT GGTGACCGTG CCCTCCAGCA GCTTGGGCAC GAAGACCTAC |
| 601 | ACCTGCAACG TAGATCACAA GCCCAGCAAC ACCAAGGTGG ACAAGAGAGT TGAGTCCAAA |
| 661 | TATGGTCCCC CATGCCCACC GTGCCCAGCA CCTGAGTTCC TGGGGGGACC ATCAGTCTTC |
| 721 | CTGTTCCCCC CAAAACCCAA GGACACTCTC ATGATCTCCC GGACCCCTGA GGTCACGTGC |
| 781 | GTGGTGGTGG ACGTGAGCCA GGAAGACCCC GAGGTCCAGT TCAACTGGTA CGTGGATGGC |
| 841 | GTGGAGGTGC ATAATGCCAA GACAAAGCCG CGGGAAGAGC AGTTCCAGAG CACGTACCGT |
| 901 | GTGGTCAGCG TCCTCACCGT CCTGCACCAG GACTGGCTGA ACGGCAAGGA GTACAAGTGC |
| 961 | AAGGTCTCCA ACAAAGGCCT CCCGTCCTCC ATCGAGAAAA CCATCTCCAA AGCCAAAGGG |
| 1021 | CAGCCCCGAG AGCCACAAGT GTACACCCTG CCCCCATCCC GGGATGAGCT GACCAAGAAC |
| 1081 | CAGGTCAGCC TGACCTGCCT GGTCAAAGGC TTCTATCCCA GCGACATCGC CGTGGAGTGG |
| 1141 | GAGAGCAATG GGCAGCCGGA GAACAACTAC AAGACCACGC CTCCCGTGTT GGACTCCGAC |
| 1201 | GGCTCCTTCT TCCTCTACAG CAAGCTCACC GTGGACAAGA GCAGGTGGCA GCAGGGGAAC |
| 1261 | GTCTTCTCAT GCTCCGTGAT GCATGAGGCT CTGCACAACC ACTACACGCA GAAGAGCCTC |
| 1321 | TCCCTGTCTC CCGGTTGA |
| Nucleic Acid Sequence of Heavy Chain Based on VH3: | |
| (SEQ ID NO: 86) | |
| 1 | GAGGTGCAGG TGGTGGAGTC CGGCGGCGGC CTGGTGAAGC CCGGCGGCTC CCTGAGGCTG |
| 61 | TCCTGCAAGG CCTCCGGCTA CACCTTCACC TCCTACTGGA TGCACTGGGT GAAGCAGGCC |
| 121 | CCCGGCAAGG GCCTGGAGTG GGTGGGCGCC ATCTACCCCG GGAATTCTGA CACCTCCTAC |
| 181 | AACCAGAAGT TCAAGGGCAA GTTCACCCTG TCCGCCGTGA CCTCCTCCAA CACCGCCTAC |
| 241 | CTGCAGATGA ACTCCCTGAA GACCGAGGAC ACCGCCGTGT ACTACTGCAC CACCACCACC |
| 301 | TACGGCTACG ACTGGTTCGC CTACTGGGGC CAGGGCACCC TGGTGACCGT CTCGAGCGCC |
| 361 | TCCACCAAGG GCCCATCCGT CTTCCCCCTG GCGCCCTGCT CCAGATCTAC CTCCGAGAGC |
| 421 | ACAGCCGCCC TGGGCTGCCT GGTCAAGGAC TACTTCCCCG AACCGGTGAC GGTGTCGTGG |
| 481 | AACTCAGGCG CCCTGACCAG CGGCGTGCAC ACCTTCCCGG CTGTCCTACA GTCCTCAGGA |
| 541 | CTCTACTCCC TCAGCAGCGT GGTGACCGTG CCCTCCAGCA GCTTGGGCAC GAAGACCTAC |
| 601 | ACCTGCAACG TAGATCACAA GCCCAGCAAC ACCAAGGTGG ACAAGAGAGT TGAGTCCAAA |
| 661 | TATGGTCCCC CATGCCCACC GTGCCCAGCA CCTGAGTTCC TGGGGGGACC ATCAGTCTTC |
| 721 | CTGTTCCCCC CAAAACCCAA GGACACTCTC ATGATCTCCC GGACCCCTGA GGTCACGTGC |
| 781 | GTGGTGGTGG ACGTGAGCCA GGAAGACCCC GAGGTCCAGT TCAACTGGTA CGTGGATGGC |
| 841 | GTGGAGGTGC ATAATGCCAA GACAAAGCCG CGGGAAGAGC AGTTCCAGAG CACGTACCGT |
| 901 | GTGGTCAGCG TCCTCACCGT CCTGCACCAG GACTGGCTGA ACGGCAAGGA GTACAAGTGC |
| 961 | AAGGTCTCCA ACAAAGGCCT CCCGTCCTCC ATCGAGAAAA CCATCTCCAA AGCCAAAGGG |
| 1021 | CAGCCCCGAG AGCCACAAGT GTACACCCTG CCCCCATCCC GGGATGAGCT GACCAAGAAC |
| 1081 | CAGGTCAGCC TGACCTGCCT GGTCAAAGGC TTCTATCCCA GCGACATCGC CGTGGAGTGG |
| 1141 | GAGAGCAATG GGCAGCCGGA GAACAACTAC AAGACCACGC CTCCCGTGTT GGACTCCGAC |
| 1201 | GGCTCCTTCT TCCTCTACAG CAAGCTCACC GTGGACAAGA GCAGGTGGCA GCAGGGGAAC |
| 1261 | GTCTTCTCAT GCTCCGTGAT GCATGAGGCT CTGCACAACC ACTACACGCA GAAGAGCCTC |
| 1321 | TCCCTGTCTC CCGGTTGA |
| Nucleic Acid Sequence of Heavy Chain Based on VH4: | |
| (SEQ ID NO: 87) | |
| 1 | GAGGTGCAGG TGGTGCAGTC CGGCGGCGGC CTGGTGAAGC CCGGCGAGTC CCTGAGGCTG |
| 61 | TCCTGCAAGG CCTCCGGCTA CACCTTCACC TCCTACTGGA TGCACTGGGT GAAGCAGGCC |
| 121 | CCCGGCAAGG GCCTGGAGTG GGTGGGCGCC ATCTACCCCG GGAATTCTGA CACCTCCTAC |
| 181 | AACCAGAAGT TCAAGGGCAA GTTCACCATC TCCGCCGACA CCTCCCCCAA CACCGCCTAC |
| 241 | CTGCAGATGA ACTCCCTGAA GACCGAGGAC ACCGCCGTGT ACTACTGCAC CACCACCACC |
| 301 | TACGGCTACG ACTGGTTCGC CTACTGGGGC CAGGGCACCC TGGTGACCGT CTCGAGCGCC |
| 361 | TCCACCAAGG GCCCATCCGT CTTCCCCCTG GCGCCCTGCT CCAGATCTAC CTCCGAGAGC |
| 421 | ACAGCCGCCC TGGGCTGCCT GGTCAAGGAC TACTTCCCCG AACCGGTGAC GGTGTCGTGG |
| 481 | AACTCAGGCG CCCTGACCAG CGGCGTGCAC ACCTTCCCGG CTGTCCTACA GTCCTCAGGA |
| 541 | CTCTACTCCC TCAGCAGCGT GGTGACCGTG CCCTCCAGCA GCTTGGGCAC GAAGACCTAC |
| 601 | ACCTGCAACG TAGATCACAA GCCCAGCAAC ACCAAGGTGG ACAAGAGAGT TGAGTCCAAA |
| 661 | TATGGTCCCC CATGCCCACC GTGCCCAGCA CCTGAGTTCC TGGGGGGACC ATCAGTCTTC |
| 721 | CTGTTCCCCC CAAAACCCAA GGACACTCTC ATGATCTCCC GGACCCCTGA GGTCACGTGC |
| 781 | GTGGTGGTGG ACGTGAGCCA GGAAGACCCC GAGGTCCAGT TCAACTGGTA CGTGGATGGC |
| 841 | GTGGAGGTGC ATAATGCCAA GACAAAGCCG CGGGAAGAGC AGTTCCAGAG CACGTACCGT |
| 901 | GTGGTCAGCG TCCTCACCGT CCTGCACCAG GACTGGCTGA ACGGCAAGGA GTACAAGTGC |
| 961 | AAGGTCTCCA ACAAAGGCCT CCCGTCCTCC ATCGAGAAAA CCATCTCCAA AGCCAAAGGG |
| 1021 | CAGCCCCGAG AGCCACAAGT GTACACCCTG CCCCCATCCC GGGATGAGCT GACCAAGAAC |
| 1081 | CAGGTCAGCC TGACCTGCCT GGTCAAAGGC TTCTATCCCA GCGACATCGC CGTGGAGTGG |
| 1141 | GAGAGCAATG GGCAGCCGGA GAACAACTAC AAGACCACGC CTCCCGTGTT GGACTCCGAC |
| 1201 | GGCTCCTTCT TCCTCTACAG CAAGCTCACC GTGGACAAGA GCAGGTGGCA GCAGGGGAAC |
| 1261 | GTCTTCTCAT GCTCCGTGAT GCATGAGGCT CTGCACAACC ACTACACGCA GAAGAGCCTC |
| 1321 | TCCCTGTCTC CCGGTTGA |
| Nucleic Acid Sequence of Heavy Chain Based on VH5: | |
| (SEQ ID NO: 88) | |
| 1 | GAGGTGCAGG TGCAGCAGTC CGGCGGCGGC CTGGTGAAGC CCGGCGGCTC CCTGAGGCTG |
| 61 | TCCTGCAAGG CCTCCGGCTA CACCTTCACC TCCTACTGGA TGCACTGGGT GAAGCAGGCC |
| 121 | CCCGGCAAGG GCCTGGAGTG GGTGGGCGCC ATCTACCCCG GGAATTCTGA CACCTCCTAC |
| 181 | AACCAGAAGT TCAAGGGCAA GGCCACCCTG TCCGCCGTGA CCTCCCCCAA CACCGCCTAC |
| 241 | CTGCAGATGA ACTCCCTGAA GACCGAGGAC ACCGCCGTGT ACTACTGCAC CACCACCACC |
| 301 | TACGGCTACG ACTGGTTCGC CTACTGGGGC CAGGGCACCC TGGTGACCGT CTCGAGCGCC |
| 361 | TCCACCAAGG GCCCATCCGT CTTCCCCCTG GCGCCCTGCT CCAGATCTAC CTCCGAGAGC |
| 421 | ACAGCCGCCC TGGGCTGCCT GGTCAAGGAC TACTTCCCCG AACCGGTGAC GGTGTCGTGG |
| 481 | AACTCAGGCG CCCTGACCAG CGGCGTGCAC ACCTTCCCGG CTGTCCTACA GTCCTCAGGA |
| 541 | CTCTACTCCC TCAGCAGCGT GGTGACCGTG CCCTCCAGCA GCTTGGGCAC GAAGACCTAC |
| 601 | ACCTGCAACG TAGATCACAA GCCCAGCAAC ACCAAGGTGG ACAAGAGAGT TGAGTCCAAA |
| 661 | TATGGTCCCC CATGCCCACC GTGCCCAGCA CCTGAGTTCC TGGGGGGACC ATCAGTCTTC |
| 721 | CTGTTCCCCC CAAAACCCAA GGACACTCTC ATGATCTCCC GGACCCCTGA GGTCACGTGC |
| 781 | GTGGTGGTGG ACGTGAGCCA GGAAGACCCC GAGGTCCAGT TCAACTGGTA CGTGGATGGC |
| 841 | GTGGAGGTGC ATAATGCCAA GACAAAGCCG CGGGAAGAGC AGTTCCAGAG CACGTACCGT |
| 901 | GTGGTCAGCG TCCTCACCGT CCTGCACCAG GACTGGCTGA ACGGCAAGGA GTACAAGTGC |
| 961 | AAGGTCTCCA ACAAAGGCCT CCCGTCCTCC ATCGAGAAAA CCATCTCCAA AGCCAAAGGG |
| 1021 | CAGCCCCGAG AGCCACAAGT GTACACCCTG CCCCCATCCC GGGATGAGCT GACCAAGAAC |
| 1081 | CAGGTCAGCC TGACCTGCCT GGTCAAAGGC TTCTATCCCA GCGACATCGC CGTGGAGTGG |
| 1141 | GAGAGCAATG GGCAGCCGGA GAACAACTAC AAGACCACGC CTCCCGTGTT GGACTCCGAC |
| 1201 | GGCTCCTTCT TCCTCTACAG CAAGCTCACC GTGGACAAGA GCAGGTGGCA GCAGGGGAAC |
| 1261 | GTCTTCTCAT GCTCCGTGAT GCATGAGGCT CTGCACAACC ACTACACGCA GAAGAGCCTC |
| 1321 | TCCCTGTCTC CCGGTTGA |
| Nucleic Acid Sequence of Heavy Chain Based on VH6: | |
| (SEQ ID NO: 89) | |
| 1 | GAGGTGCAGG TGGTGGAGTC CGGCGGCGGC CTGGTGAAGC CCGGCGGCTC CCTGAGGCTG |
| 61 | TCCTGCGCCG CCTCCGGCTA CACCTTCACC TCCTACTGGA TGCACTGGGT GAGGCAGGCC |
| 121 | CCCGGCAAGG GCCTGGAGTG GGTGGGCAGG ATCAAGTCCA AGACCGACGG CGGTACCACC |
| 181 | GACTACGCCG CCCCCGTGAA GGGCAGGTTC ACCATCTCCG CCGACACCTC CAAGAACACC |
| 241 | CTGTACCTGC AGATGAACTC CCTGAAGACC GAGGACACCG CCGTGTACTA CTGCACCACC |
| 301 | ACCACCTACG GCTACGACTG GTTCGCCTAC TGGGGCCAGG GCACCCTGGT GACCGTCTCG |
| 361 | AGCGCCTCCA CCAAGGGCCC ATCCGTCTTC CCCCTGGCGC CCTGCTCCAG ATCTACCTCC |
| 421 | GAGAGCACAG CCGCCCTGGG CTGCCTGGTC AAGGACTACT TCCCCGAACC GGTGACGGTG |
| 481 | TCGTGGAACT CAGGCGCCCT GACCAGCGGC GTGCACACCT TCCCGGCTGT CCTACAGTCC |
| 541 | TCAGGACTCT ACTCCCTCAG CAGCGTGGTG ACCGTGCCCT CCAGCAGCTT GGGCACGAAG |
| 601 | ACCTACACCT GCAACGTAGA TCACAAGCCC AGCAACACCA AGGTGGACAA GAGAGTTGAG |
| 661 | TCCAAATATG GTCCCCCATG CCCACCGTGC CCAGCACCTG AGTTCCTGGG GGGACCATCA |
| 721 | GTCTTCCTGT TCCCCCCAAA ACCCAAGGAC ACTCTCATGA TCTCCCGGAC CCCTGAGGTC |
| 781 | ACGTGCGTGG TGGTGGACGT GAGCCAGGAA GACCCCGAGG TCCAGTTCAA CTGGTACGTG |
| 841 | GATGGCGTGG AGGTGCATAA TGCCAAGACA AAGCCGCGGG AAGAGCAGTT CCAGAGCACG |
| 901 | TACCGTGTGG TCAGCGTCCT CACCGTCCTG CACCAGGACT GGCTGAACGG CAAGGAGTAC |
| 961 | AAGTGCAAGG TCTCCAACAA AGGCCTCCCG TCCTCCATCG AGAAAACCAT CTCCAAAGCC |
| 1021 | AAAGGGCAGC CCCGAGAGCC ACAAGTGTAC ACCCTGCCCC CATCCCGGGA TGAGCTGACC |
| 1081 | AAGAACCAGG TCAGCCTGAC CTGCCTGGTC AAAGGCTTCT ATCCCAGCGA CATCGCCGTG |
| 1141 | GAGTGGGAGA GCAATGGGCA GCCGGAGAAC AACTACAAGA CCACGCCTCC CGTGTTGGAC |
| 1201 | TCCGACGGCT CCTTCTTCCT CTACAGCAAG CTCACCGTGG ACAAGAGCAG GTGGCAGCAG |
| 1261 | GGGAACGTCT TCTCATGCTC CGTGATGCAT GAGGCTCTGC ACAACCACTA CACGCAGAAG |
| 1321 | AGCCTCTCCC TGTCTCCCGG TTGA |

The following are exemplary nucleic acid sequences encoding light chains based on the humanized ALULA VL0-VL4 designs described above. These light chain have a constant region comprising the kappa light chain constant region of human IgG4.

| Nucleic Acid Sequence of Heavy Chain Based on VL0: | |
|---|---|
| (SEQ ID NO: 90) | |
| 1 | GACATCCAGA TGACCCAGTC CCCCTCCTCC GTGTCCGCCT CCGTGGGCGA CAGGGTGACC |
| 61 | ATCACCTGCA AGTCCTCCCA GTCCGTGCTG TACAGCTCCA ACCAGAAGAA CTACCTGGCC |
| 121 | TGGTACCAGC AGAAGCCCGG CAAGGCCCCC AAGCTGCTGA TCTACTGGGC CTCCACCAGG |
| 181 | GAGTCCGGCG TGCCCTCCAG GTTCTCCGGC TCCGGCTCCG GCACCGACTT CACCCTGACC |
| 241 | ATCTCCTCCC TGCAGCCCGA GGACTTCGCC ACCTACTACT GCCACCAGTA CCTCTCGAGC |
| 301 | CTGACCTTCG GCCAGGGCAC CAAGGTGGAG ATCAAGCGAA CTGTGGCTGC ACCATCTGTC |
| 361 | TTCATCTTCC CGCCATCTGA TGAGCAGTTG AAATCTGGAA CTGCCTCTGT TGTGTGCCTG |
| 421 | CTGAATAACT TCTATCCCAG AGAGGCCAAA GTACAGTGGA AGGTGGATAA CGCCCTCCAA |
| 481 | TCGGGTAACT CCCAGGAGAG TGTCACAGAG CAGGACAGCA AGGACAGCAC CTACAGCCTC |
| 541 | AGCAGCACCC TGACGCTGAG CAAAGCAGAC TACGAGAAAC ACAAAGTCTA CGCCTGCGAA |
| 601 | GTCACCCATC AGGGCCTGAG CTCGCCCGTC ACAAAGAGCT TCAACAGGGG AGAGTGTTGA |
| Nucleic Acid Sequence of Heavy Chain Based on VL1: | |
| (SEQ ID NO: 91) | |
| 1 | GACATCCAGA TGACCCAGTC CCCCTCCTCC CTGTCCGCCT CCGTGGGCGA CAGGGTGACC |
| 61 | ATGACCTGCA AGTCCTCCCA GTCCGTGCTG TACAGCTCCA ACCAGAAGAA CTACCTGGCC |
| 121 | TGGTACCAGC AGAAGCCCGG CAAGGCCCCC AAGCTGCTGA TCTACTGGGC CTCCACCAGG |
| 181 | GAGTCCGGCG TGCCCGACAG GTTCTCCGGC TCCGGCTCCG GCACCGACTT CACCCTGACC |
| 241 | ATCTCCTCCC TGCAGCCCGA GGACTTCGCC ACCTACTACT GCCACCAGTA CCTCTCGAGC |
| 301 | CTGACCTTCG GCCAGGGCAC CAAGCTGGAG ATCAAGCGAA CTGTGGCTGC ACCATCTGTC |
| 361 | TTCATCTTCC CGCCATCTGA TGAGCAGTTG AAATCTGGAA CTGCCTCTGT TGTGTGCCTG |
| 421 | CTGAATAACT TCTATCCCAG AGAGGCCAAA GTACAGTGGA AGGTGGATAA CGCCCTCCAA |
| 481 | TCGGGTAACT CCCAGGAGAG TGTCACAGAG CAGGACAGCA AGGACAGCAC CTACAGCCTC |
| 541 | AGCAGCACCC TGACGCTGAG CAAAGCAGAC TACGAGAAAC ACAAAGTCTA CGCCTGCGAA |
| 601 | GTCACCCATC AGGGCCTGAG CTCGCCCGTC ACAAAGAGCT TCAACAGGGG AGAGTGTTGA |
| Nucleic Acid Sequence of Heavy Chain Based on VL2: | |
| (SEQ ID NO: 92) | |
| 1 | AACATCCAGA TGACCCAGTC CCCCTCCTCC CTGTCCGCCT CCGTGGGCGA CAGGGTGACC |
| 61 | ATGTCCTGCA AGTCCTCCCA GTCCGTGCTG TACAGCTCCA ACCAGAAGAA CTACCTGGCC |
| 121 | TGGTACCAGC AGAAGCCCGG CAAGTCCCCC AAGCTGCTGA TCTACTGGGC CTCCACCAGG |
| 181 | GAGTCCGGCG TGCCCGACAG GTTCACCGGC TCCGGCTCCG GCACCGACTT CACCCTGACC |
| 241 | ATCTCCTCCC TGCAGCCCGA GGACTTCGCC ACCTACTACT GCCACCAGTA CCTCTCGAGC |
| 301 | CTGACCTTCG GCCAGGGCAC CAAGCTGGAG ATCAAGCGAA CTGTGGCTGC ACCATCTGTC |
| 361 | TTCATCTTCC CGCCATCTGA TGAGCAGTTG AAATCTGGAA CTGCCTCTGT TGTGTGCCTG |
| 421 | CTGAATAACT TCTATCCCAG AGAGGCCAAA GTACAGTGGA AGGTGGATAA CGCCCTCCAA |
| 481 | TCGGGTAACT CCCAGGAGAG TGTCACAGAG CAGGACAGCA AGGACAGCAC CTACAGCCTC |
| 541 | AGCAGCACCC TGACGCTGAG CAAAGCAGAC TACGAGAAAC ACAAAGTCTA CGCCTGCGAA |
| 601 | GTCACCCATC AGGGCCTGAG CTCGCCCGTC ACAAAGAGCT TCAACAGGGG AGAGTGTTGA |
| Nucleic Acid Sequence of Heavy Chain Based on VL3: | |
| (SEQ ID NO: 93) | |
| 1 | GACATCCAGA TGACCCAGTC CCCCTCCTCC CTGACCGTGT CCGTGGGCGA CAGGGTGACC |
| 61 | ATGTCCTGCA AGTCCTCCCA GTCCGTGCTG TACAGCTCCA ACCAGAAGAA CTACCTGGCC |
| 121 | TGGTACCAGC AGAAGCCCGG CAAGTCCCCC AAGCTGCTGA TCTACTGGGC CTCCACCAGG |
| 181 | GAGTCCGGCG TGCCCGACAG GTTCACCGGC TCCGGCTCCG GCACCGACTT CACCCTGACC |
| 241 | ATCTCCTCCG TGCAGCCCGA GGACTTCGCC ACCTACTACT GCCACCAGTA CCTCTCGAGC |
| 301 | CTGACCTTCG GCGCCGGCAC CAAGCTGGAG ATCAAGCGAA CTGTGGCTGC ACCATCTGTC |
| 361 | TTCATCTTCC CGCCATCTGA TGAGCAGTTG AAATCTGGAA CTGCCTCTGT TGTGTGCCTG |
| 421 | CTGAATAACT TCTATCCCAG AGAGGCCAAA GTACAGTGGA AGGTGGATAA CGCCCTCCAA |
| 481 | TCGGGTAACT CCCAGGAGAG TGTCACAGAG CAGGACAGCA AGGACAGCAC CTACAGCCTC |
| 541 | AGCAGCACCC TGACGCTGAG CAAAGCAGAC TACGAGAAAC ACAAAGTCTA CGCCTGCGAA |
| 601 | GTCACCCATC AGGGCCTGAG CTCGCCCGTC ACAAAGAGCT TCAACAGGGG AGAGTGTTGA |
| Nucleic Acid Sequence of Heavy Chain Based on VL4: | |
| (SEQ ID NO: 94) | |
| 1 | GACATCCAGA TGACCCAGTC CCCCTCCTCC CTGTCCGCCT CCGTGGGCGA CAGGGTGACC |
| 61 | ATGACCTGCA AGTCCTCCCA GTCCGTGCTG TACAGCTCCA ACCAGAAGAA CTACCTGGCC |
| 121 | TGGTACCAGC AGAAGCCCGG CAAGGCCCCC AAGCTGCTGA TCTACGCCGC CTCCTCCCTG |
| 181 | CAGTCCGGCG TGCCCTCCAG GTTCTCCGGC TCCGGCTCCG GCACCGACTT CACCCTGACC |
| 241 | ATCTCCTCCC TGCAGCCCGA GGACTTCGCC ACCTACTACT GCCACCAGTA CCTCTCGAGC |
| 301 | CTGACCTTCG GCCAGGGCAC CAAGCTGGAG ATCAAGCGAA CTGTGGCTGC ACCATCTGTC |
| 361 | TTCATCTTCC CGCCATCTGA TGAGCAGTTG AAATCTGGAA CTGCCTCTGT TGTGTGCCTG |
| 421 | CTGAATAACT TCTATCCCAG AGAGGCCAAA GTACAGTGGA AGGTGGATAA CGCCCTCCAA |
| 481 | TCGGGTAACT CCCAGGAGAG TGTCACAGAG CAGGACAGCA AGGACAGCAC CTACAGCCTC |
| 541 | AGCAGCACCC TGACGCTGAG CAAAGCAGAC TACGAGAAAC ACAAAGTCTA CGCCTGCGAA |
| 601 | GTCACCCATC AGGGCCTGAG CTCGCCCGTC ACAAAGAGCT TCAACAGGGG AGAGTGTTGA |

### Example 10: FACS Binding With MAbs/Fabs

This experiment was carried out to confirm the affinity of ALULA-H4/L2-IgG4.P(S228P)/IgG1(N297Q) for cell surface expressed αvβ5 stably expressed on murine BaF3 cells. Binding curves were generated using a lower number of cells to ensure that antibody was not simply titrating the receptors on the cell. Using 10,000 cells/experiment, K_{D} values were determined to be 0.08±0.02 nM for the mAb, and 0.42±0.03 for the monovalent Fab.

### Example 11: Stability of Humanized ALULA Constructs

The thermal stability of purified humanized ALULA antibodies VH2/VL2, VH4/VL0, VH4/VL2, and VH5/VL2, all containing the aglycosylated IgG4.P (S228P)/IgG1 (N297Q) domain, was determined using differential scanning calorimetry (DSC) (see, Table 5). These were compared to the corresponding values for a reference humanized αvβ5 design (Design-Reference H1 and Design-Reference L1; see Example 1) expressed on the same Fc domain.

For each antibody, three distinct transitions were observed, corresponding to the unfolding of the CH2 and CH3 regions of the constant domain (at 57-59° C. and 84-85° C., respectively), and the unfolding of the Fab region comprising the variable domains and CH1 constant domain (at 69-73° C.). The Fab region of versions VH4/VL2, VH4/VL0, and VH2/VL2 each had a slightly higher melting temperature (Tm), indicative of higher thermal stability, than the VH5/VL2 or the reference humanized αvβ5 design antibody. Of note, both VH4 and VH2 heavy chains contain a glutamic acid residue at position 16, while VH5 has a glycine at this position and the reference humanized αvβ5 design has an alanine. Monovalent Fab fragments were generated by papain cleavage of the Agly-IgG1(T299A) version of each mAb, and the thermal stability of the isolated Fab was measured. The VH2/VL2, VH4/VL0, and VH4/VL2 Fabs had Tm values between 75.4-76.3, while the Tm of the VH5/VL2 Fab was 72.1, consistent with the differences in thermal stability observed in this region within the intact mAbs.

TABLE 5
Thermal Stability of Antibodies Determined Using DSC

| | Tm(° C.) | | |
|---|---|---|---|
| | | Fab (V_{II}V_{L} + | |
| Antibody | CH2 | CH1) | CH3 |
| Reference humanized αvβ5 Design hIgG4P | 57.1 | 70.7 | 84.2 |
| agly/hIgG1(CH3) | | | |
| H2L2 hIgG4P agly/hIgG1(CH3) | 57.6 | 72.7 | 84.9 |
| H4L0 hIgG4P agly/hIgG1(CH3) | 56.5 | 72.8 | 84.8 |
| H4L2 hIgG4P agly/hIgG1(CH3) | 57.0 | 72.4 | 84.8 |
| H5L2 hIgG4P agly/hIgG1(CH3) | 59.9 | 69.3 | 84.3 |
| H2L2 hIgG1 Fab | — | 75.9 | — |
| H4L0 hIgG1 Fab | — | 76.3 | — |
| H4L2 hIgG1 Fab | — | 75.4 | — |
| H5L2 hIgG1 Fab | — | 72.1 | — |

### Example 12: FACS Direct Binding Assay for H4/L2

The binding of the H4/L2 antibody (SEQ ID NOs.: 69 and 70) to αvβ5 expressed on the cell surface was determined using flow cytometry on BaF3 cells stably co-transfected with human αv and human β5 integrin subunits. BaF3 cells are a murine IL-3 dependent hematopoietic cell line of indeterminate origin. These cells endogenously express mouse alpha-V (as well as alpha-4, alpha-5, and beta-1 integrins) but no beta-5 or beta-6 integrin.

The H4/L2 antibody was added to cells in FACS Buffer (PBS, 1% BSA, 1 mM CaCl₂, 1 mM MgCl₂) for 30 minutes on ice. After washing, bound antibody was detected with an anti-human-Alexa Fluor® 488 secondary antibody. Cells fixed in 1% paraformaldehyde were collected on a FACS Calibur and mean fluorescence intensity was analyzed using FlowJo software.

The H4/L2 antibody bound specifically to αvβ5-BaF3 cells, with no detectable binding to parental BaF3 cells. Binding of the H4/L2 antibody was dose-dependent, with an EC₅₀ of 3 nM.

### Example 13: Apparent Binding Affinity for H4/L2 Antibody

The apparent binding affinity of the H4/L2 antibody (SEQ ID NOs.: 69 and 70) for immobilized human αvβ5 (hs αvβ5) was determined using an enzyme-linked immunosorbent assay (ELISA). Purified hsαvβ5 protein was coated directly on a 96-well ELISA plate at 2 μg/mL, and H4/L2 antibody binding was detected using a horseradish peroxidase conjugated donkey anti-human IgG polyclonal antibody. In this format, the EC₅₀ of the H4/L2 antibody for αvβ5 was about 65 pM.

### Example 14: Manufacturability Assessments

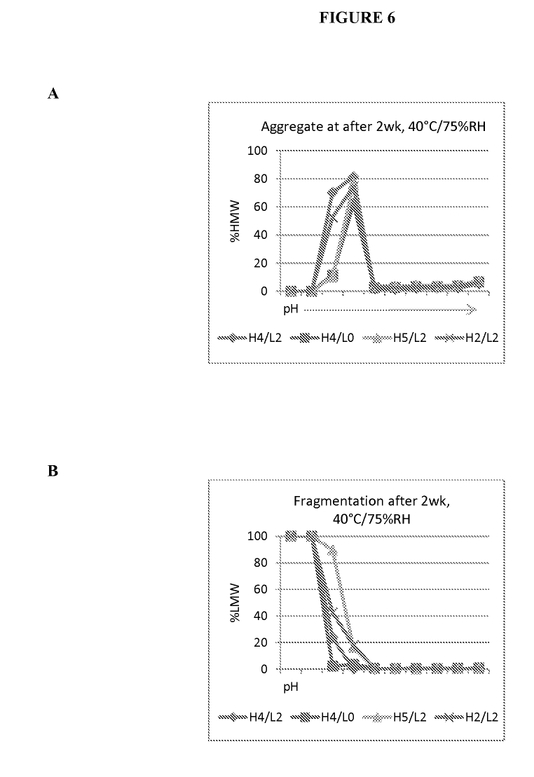

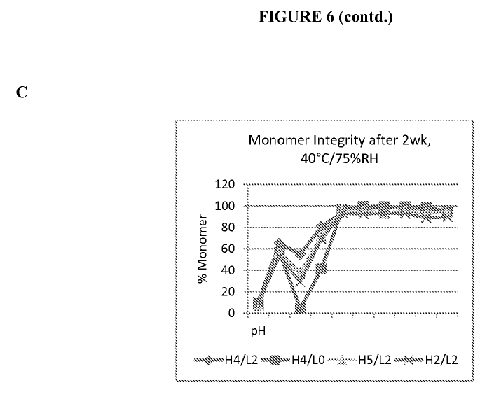

The H4/L2 antibody, along with three others (H4/L0, H5/L2, and H2/L2), was assessed for stability in situations key for developability and manufacturability. As part of the manufacturing process for biological therapeutics, compounds are often subjected to prolonged incubation at low pH, in order to inactivate any potentially contaminating viruses. Standard purification procedures for monoclonal antibodies may also incorporate low pH conditions. Thus, stability at low pH can be an important attribute. Screening across a wide range of pH over a course of weeks indicated that each of these constructs possess reduced stability by multiple measures at low pH (FIG. 6A). However, H4/L2 demonstrated less fragmentation than all but one of the other constructs (FIG. 6B). While it and others exhibited increased aggregation in this pH range, it maintained a higher level of monomer integrity versus all other constructs (FIG. 6C).

Conformational stability was assessed via Differential Scanning calorimetry (DSC) in multiple studies over a wide pH range, in multiple buffer systems, and with the inclusion of common excipients. H4/L2 matched the rest of the constructs with respect to conformational stability, with the exception of H5/L2 which was consistently less stable in this measure than all other constructs.

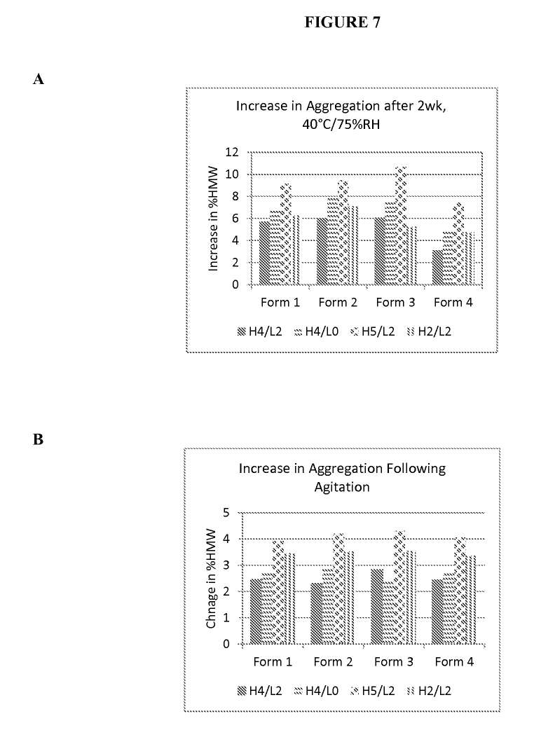

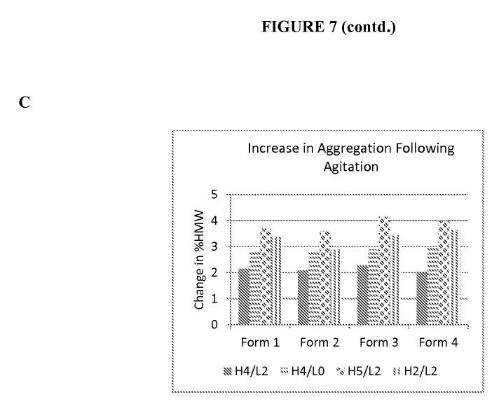

The stability of a protein to common stresses (e.g., elevated temperatures, agitation, or freeze-thaw) is highly desirable with respect to both manufacturability and projected shelf-life. Stability under accelerated stress conditions is frequently used as a tool to attempt prediction of the real time long-term stability of a protein stored under optimal conditions. In the case of biologics normally stored at 5° C., accelerated conditions often consist of 25° C./60% RH (relative humidity) and 40° C./75% RH. At high concentration, in accelerated conditions (40° C./75% RH) all constructs showed increased aggregation behavior. However, in the majority of formulations and pHs, H4/L2 was the most resistant to aggregation at accelerated temperatures (FIG. 7A). This effect was also observed in response to both agitation (FIG. 7B) and freeze-thaw (FIG. 7C). Stability to stresses such as high temperature, agitation, and freeze-thaw is highly desirable with respect to both protein manufacturability and projected shelf life.

### Example 15: Efficacy of Anti-αvβ5 Antibody in Renal Ischemia Model

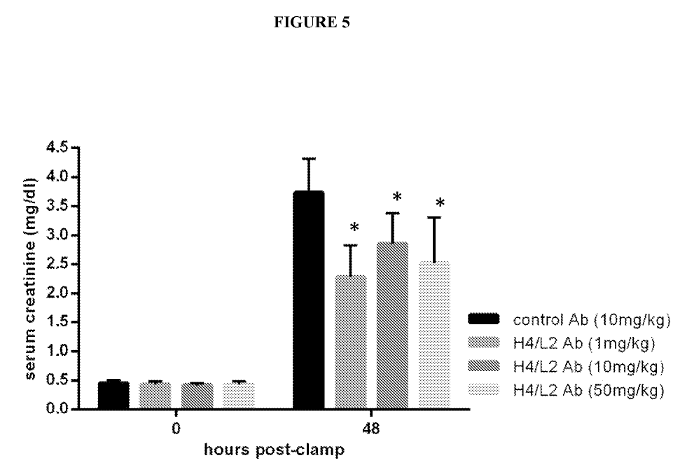

The rat unilateral ischemic clamp model was used to study the efficacy of a humanized anti-αvβ5 antibody in the prevention of renal ischemia. Five to six rats were treated with a single 1, 10, or 50 mg/kg administration of H4/L2 antibody (comprised of SEQ ID NOs.: 69 and 70) or control antibody (1E6) at 6 hours prior to clamping the renal artery. Blood was collected at baseline and 48 hours after clamping to assess serum creatinine levels. A single dose of anti-αvβ5 antibody was able to significantly attenuate the ischemia-induced rise in serum creatinine post-surgery (FIG. 5; *P<0.05 vs. control antibody; Error bars=SD).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-22. (canceled)

23. An antibody or antigen-binding fragment thereof that specifically binds to αvβ5, wherein the antibody or the antigen-binding fragment thereof comprises:
a heavy chain variable region that comprises the amino acid sequence set forth in SEQ ID NO:1 and a light chain variable region that comprises the amino acid sequence set forth in SEQ ID NO:8, 9, 10, 11, or 12;
a heavy chain variable region that comprises the amino acid sequence set forth in SEQ ID NO:2 and a light chain variable region that comprises the amino acid sequence set forth in SEQ ID NO:8, 9, 10, 11, or 12;
a heavy chain variable region that comprises the amino acid sequence set forth in SEQ ID NO:3 and a light chain variable region that comprises the amino acid sequence set forth in SEQ ID NO:8, 9, 10, 11, or 12;
a heavy chain variable region that comprises the amino acid sequence set forth in SEQ ID NO:4 and a light chain variable region that comprises the amino acid sequence set forth in SEQ ID NO:8, 9, 10, 11, or 12;
a heavy chain variable region that comprises the amino acid sequence set forth in SEQ ID NO:5 and a light chain variable region that comprises the amino acid sequence set forth in SEQ ID NO:8, 9, 10, 11, or 12;
a heavy chain variable region that comprises the amino acid sequence set forth in SEQ ID NO:6 and a light chain variable region that comprises the amino acid sequence set forth in SEQ ID NO:8, 9, 10, 11, or 12; or
a heavy chain variable region that comprises the amino acid sequence set forth in SEQ ID NO:7 and a light chain variable region that comprises the amino acid sequence set forth in SEQ ID NO:8, 9, 10, 11, or 12.

24. The antibody or the antigen-binding fragment thereof of claim 23, wherein the antibody or the antigen-binding fragment thereof comprises:
a heavy chain variable region that comprises the amino acid sequence set forth in SEQ ID NO:3 and a light chain variable region that comprises the amino acid sequence set forth in SEQ ID NO:10;
a heavy chain variable region that comprises the amino acid sequence set forth in SEQ ID NO:5 and a light chain variable region that comprises the amino acid sequence set forth in SEQ ID NO:8;
a heavy chain variable region that comprises the amino acid sequence set forth in SEQ ID NO:5 and a light chain variable region that comprises the amino acid sequence set forth in SEQ ID NO:9;
a heavy chain variable region that comprises the amino acid sequence set forth in SEQ ID NO:5 and a light chain variable region that comprises the amino acid sequence set forth in SEQ ID NO:10; or
a heavy chain variable region that comprises the amino acid sequence set forth in SEQ ID NO:6 and a light chain variable region that comprises the amino acid sequence set forth in SEQ ID NO:10.

25. The antibody or the antigen-binding fragment thereof of claim 23, wherein the antibody has an isotype selected from the group consisting of IgG1, IgG2, IgG3, and IgG4.

26. The antibody or the antigen-binding fragment thereof of claim 23, wherein the antibody comprises a CH1 domain and a CH2 domain from an IgG antibody of the IgG4 isotype and a CH3 domain from an IgG antibody of the IgG1 isotype.

27. The antibody or the antigen-binding fragment thereof of claim 23, wherein the antibody comprises a CH1 domain and a CH2 domain from an IgG antibody of the IgG4 isotype and a CH3 domain from an IgG antibody of the IgG1 isotype, and wherein the antibody comprises a S228P and an N297Q mutation (numbering according to Kabat).

28. The antibody or the antigen-binding fragment thereof of claim 23, wherein the antigen-binding fragment is selected from the group consisting of an Fab, an Fab′, an F(ab′)2, an Fv, a diabody, an scFv, and an sc(Fv)₂.

29. An antibody comprising the amino acid sequence set forth in
(i) SEQ ID NO:69 and 70;
(ii) SEQ ID NO:69 and 82;
(iii) SEQ ID NO:80 and 82; or
(iv) SEQ ID NO:81 and 70.

30. The antibody or the antigen-binding fragment thereof of claim 23, wherein the antibody is conjugated to a substance selected from the group consisting of a toxin, a radionuclide, a fluorescent label, polyethylene glycol, and a cytotoxic agent.

31. A pharmaceutical composition comprising the antibody or the antigen-binding fragment thereof of claim 23 and a pharmaceutically acceptable carrier.

32. A method of treating acute kidney injury in a human subject in need thereof, comprising administering to the human subject the antibody or the antigen-binding fragment thereof of claim 23.

33. A method of treating acute lung injury in a human subject in need thereof, comprising administering to the human subject the antibody or the antigen-binding fragment thereof of claim 23.

34. A method of treating stroke in a human subject in need thereof, comprising administering to the human subject the antibody or the antigen-binding fragment thereof of claim 23.

35. A method of treating ocular neovascularization disease in a human subject in need thereof, comprising administering to the human subject the antibody or the antigen-binding fragment thereof of claim 23.

36. A method of treating sepsis in a human subject in need thereof, comprising administering to the human subject the antibody or the antigen-binding fragment thereof of claim 23.

37. A method of treating myocardial infarction in a human subject in need thereof, comprising administering to the human subject the antibody or the antigen-binding fragment thereof of claim 23.

38. A method of treating pulmonary edema in a human subject in need thereof, comprising administering to the human subject the antibody or the antigen-binding fragment thereof of claim 23.

39. A method of treating lung fibrosis in a human subject in need thereof, comprising administering to the human subject the antibody or the antigen-binding fragment thereof of claim 23.

40. The method of claim 39, wherein the lung fibrosis is usual interstitial pneumonia.

41. The method of claim 39, wherein the lung fibrosis is idiopathic pulmonary fibrosis.

42. A method of treating cancer in a human subject in need thereof, comprising administering to the human subject the antibody or the antigen-binding fragment thereof of claim 23.

43. A method of inhibiting angiogenesis in a human subject in need thereof, comprising administering to the human subject the antibody or the antigen-binding fragment thereof of claim 23.

44. An isolated nucleic acid comprising a nucleotide sequence selected from the group consisting of SEQ ID NOs:95 to 102, 34, and 53 to 55.

45. (canceled)

46. An isolated nucleic acid comprising a nucleotide sequence that encodes a polypeptide comprising an amino acid sequence selected from the group consisting of SEQ ID NOs:1-7, 8-12, 69, 70, 80, 81, and 82.

47. (canceled)

48. An isolated protein encoded by the nucleic acid of claim 44.

49. A recombinant vector comprising the nucleic acid of claim 44.

50. A host cell comprising the recombinant vector of claim 49.

51. A method of preparing a humanized antibody comprising culturing a host cell comprising recombinant vectors comprising:
the nucleic acid sequences set forth in SEQ ID NOs:99 and 53;
the nucleic acid sequences set forth in SEQ ID NOs:99 and 102;
the nucleic acid sequences set forth in SEQ ID NOs:97 and 102;
the nucleic acid sequences set forth in SEQ ID NOs:100 and 53; or
the nucleic acid sequences set forth in SEQ ID NOs:99 and 34;
under conditions appropriate for expression of a humanized antibody, wherein humanized antibody chains are expressed and a humanized antibody is produced.

52. The method of claim 51, further comprising isolating the humanized antibody.

53. The method of claim 51, wherein the host cell is a CHO cell.


SEQUENCE LISTING (102 sequences)

SEQ ID NO: 1 - protein, 119 aa, Artificial Sequence
  source: /note=“Description of Artificial Sequence Synthetic polypeptide”
EVQLVESGGGLVKPGGSLRLSCAASGYTFTSYWMHWVRQAPGKGLEWVGAIYPGNSDTSYNQKFKGRFTISRDDSKNTLYLQMNSLKTEDTAVYYCTTTTYGYDWFAYWGQGTLVTVSS

SEQ ID NO: 2 - protein, 119 aa, Artificial Sequence
  source: /note=“Description of Artificial Sequence Synthetic polypeptide”
EVQVVESGGGLVKPGGSLRLSCAASGYTFTSYWMHWVRQAPGKGLEWVGAIYPGNSDTSYNQKFKGRFTISADTSKNTLYLQMNSLKTEDTAVYYCTTTTYGYDWFAYWGQGTLVTVSS

SEQ ID NO: 3 - protein, 119 aa, Artificial Sequence
  source: /note=“Description of Artificial Sequence Synthetic polypeptide”
EVQVVQSGGGLVKPGESLRLSCAASGYTFTSYWMHWVKQAPGKGLEWVGAIYPGNSDTSYNQKFKGKFTISADTSSNTAYLQMNSLKTEDTAVYYCTTTTYGYDWFAYWGQGTLVTVSS

SEQ ID NO: 4 - protein, 119 aa, Artificial Sequence
  source: /note=“Description of Artificial Sequence Synthetic polypeptide”
EVQVVESGGGLVKPGGSLRLSCKASGYTFTSYWMHWVKQAPGKGLEWVGAIYPGNSDTSYNQKFKGKFTLSAVTSSNTAYLQMNSLKTEDTAVYYCTTTTYGYDWFAYWGQGTLVTVSS

SEQ ID NO: 5 - protein, 119 aa, Artificial Sequence
  source: /note=“Description of Artificial Sequence Synthetic polypeptide”
EVQVVQSGGGLVKPGESLRLSCKASGYTFTSYWMHWVKQAPGKGLEWVGAIYPGNSDTSYNQKFKGKFTISADTSPNTAYLQMNSLKTEDTAVYYCTTTTYGYDWFAYWGQGTLVTVSS

SEQ ID NO: 6 - protein, 119 aa, Artificial Sequence
  source: /note=“Description of Artificial Sequence Synthetic polypeptide”
EVQVQQSGGGLVKPGGSLRLSCKASGYTFTSYWMHWVKQAPGKGLEWVGAIYPGNSDTSYNQKFKGKATLSAVTSPNTAYLQMNSLKTEDTAVYYCTTTTYGYDWFAYWGQGTLVTVSS

SEQ ID NO: 7 - protein, 121 aa, Artificial Sequence
  source: /note=“Description of Artificial Sequence Synthetic polypeptide”
EVQVVESGGGLVKPGGSLRLSCAASGYTFTSYWMHWVRQAPGKGLEWVGRIKSKTDGGTTDYAAPVKGRFTISADTSKNTLYLQMNSLKTEDTAVYYCTTTTYGYDWFAYWGQGTLVTVSS

SEQ ID NO: 8 - protein, 112 aa, Artificial Sequence
  source: /note=“Description of Artificial Sequence Synthetic polypeptide”
DIQMTQSPSSVSASVGDRVTITCKSSQSVLYSSNQKNYLAWYQQKPGKAPKLLIYWASTRESGVPSRFSGSGSGTDFTLTISSLQPEDFATYYCHQYLSSLTFGQGTKVEIK

SEQ ID NO: 9 - protein, 112 aa, Artificial Sequence
  source: /note=“Description of Artificial Sequence Synthetic polypeptide”
DIQMTQSPSSLSASVGDRVTMTCKSSQSVLYSSNQKNYLAWYQQKPGKAPKLLIYWASTRESGVPDRFSGSGSGTDFTLTISSLQPEDFATYYCHQYLSSLTFGQGTKLEIK

SEQ ID NO: 10 - protein, 112 aa, Artificial Sequence
  source: /note=“Description of Artificial Sequence Synthetic polypeptide”
NIQMTQSPSSLSASVGDRVTMSCKSSQSVLYSSNQKNYLAWYQQKPGKSPKLLIYWASTRESGVPDRFTGSGSGTDFTLTISSLQPEDFATYYCHQYLSSLTFGQGTKLEIK

SEQ ID NO: 11 - protein, 112 aa, Artificial Sequence
  source: /note=“Description of Artificial Sequence Synthetic polypeptide”
DIQMTQSPSSLTVSVGDRVTMSCKSSQSVLYSSNQKNYLAWYQQKPGKSPKLLIYWASTRESGVPDRFTGSGSGTDFTLTISSVQPEDFATYYCHQYLSSLTFGAGTKLEIK

SEQ ID NO: 12 - protein, 112 aa, Artificial Sequence
  source: /note=“Description of Artificial Sequence Synthetic polypeptide”
DIQMTQSPSSLSASVGDRVTMTCKSSQSVLYSSNQKNYLAWYQQKPGKAPKLLIYAASSLQSGVPSRFSGSGSGTDFTLTISSLQPEDFATYYCHQYLSSLTFGQGTKLEIK

SEQ ID NO: 13 - protein, 5 aa, Artificial Sequence
  source: /note=“Description of Artificial Sequence Synthetic peptide”
SYWMH

SEQ ID NO: 14 - protein, 17 aa, Artificial Sequence
  source: /note=“Description of Artificial Sequence Synthetic peptide”
AIYPGNSDTSYNQKFKG

SEQ ID NO: 15 - protein, 10 aa, Artificial Sequence
  source: /note=“Description of Artificial Sequence Synthetic peptide”
TTYGYDWFAY

SEQ ID NO: 16 - protein, 17 aa, Artificial Sequence
  source: /note=“Description of Artificial Sequence Synthetic peptide”
KSSQSVLYSSNQKNYLA

SEQ ID NO: 17 - protein, 7 aa, Artificial Sequence
  source: /note=“Description of Artificial Sequence Synthetic peptide”
WASTRES

SEQ ID NO: 18 - protein, 8 aa, Artificial Sequence
  source: /note=“Description of Artificial Sequence Synthetic peptide”
HQYLSSLT

SEQ ID NO: 19 - protein, 25 aa, Artificial Sequence
  source: /note=“Description of Artificial Sequence Synthetic peptide”
EVQLVESGGGLVKPGGSLRLSCAAS

SEQ ID NO: 20 - protein, 14 aa, Artificial Sequence
  source: /note=“Description of Artificial Sequence Synthetic peptide”
WVRQAPGKGLEWVG

SEQ ID NO: 21 - protein, 32 aa, Artificial Sequence
  source: /note=“Description of Artificial Sequence Synthetic polypeptide”
RFTISRDDSKNTLYLQMNSLKTEDTAVYYCTT

SEQ ID NO: 22 - protein, 11 aa, Artificial Sequence
  source: /note=“Description of Artificial Sequence Synthetic peptide”
WGQGTLVTVSS

SEQ ID NO: 23 - protein, 25 aa, Artificial Sequence
  source: /note=“Description of Artificial Sequence Synthetic peptide”
EVQVVESGGGLVKPGGSLRLSCAAS

SEQ ID NO: 24 - protein, 32 aa, Artificial Sequence
  source: /note=“Description of Artificial Sequence Synthetic polypeptide”
RFTISADTSKNTLYLQMNSLKTEDTAVYYCTT

SEQ ID NO: 25 - protein, 25 aa, Artificial Sequence
  source: /note=“Description of Artificial Sequence Synthetic peptide”
EVQVVQSGGGLVKPGESLRLSCAAS

SEQ ID NO: 26 - protein, 14 aa, Artificial Sequence
  source: /note=“Description of Artificial Sequence Synthetic peptide”
WVKQAPGKGLEWVG

SEQ ID NO: 27 - protein, 32 aa, Artificial Sequence
  source: /note=“Description of Artificial Sequence Synthetic polypeptide”
KFTISADTSSNTAYLQMNSLKTEDTAVYYCTT

SEQ ID NO: 28 - protein, 25 aa, Artificial Sequence
  source: /note=“Description of Artificial Sequence Synthetic peptide”
EVQVVESGGGLVKPGGSLRLSCKAS

SEQ ID NO: 29 - protein, 32 aa, Artificial Sequence
  source: /note=“Description of Artificial Sequence Synthetic polypeptide”
KFTLSAVTSSNTAYLQMNSLKTEDTAVYYCTT

SEQ ID NO: 30 - protein, 25 aa, Artificial Sequence
  source: /note=“Description of Artificial Sequence Synthetic peptide”
EVQVVQSGGGLVKPGESLRLSCKAS

SEQ ID NO: 31 - protein, 32 aa, Artificial Sequence
  source: /note=“Description of Artificial Sequence Synthetic polypeptide”
KFTISADTSPNTAYLQMNSLKTEDTAVYYCTT

SEQ ID NO: 32 - protein, 25 aa, Artificial Sequence
  source: /note=“Description of Artificial Sequence Synthetic peptide”
EVQVQQSGGGLVKPGGSLRLSCKAS

SEQ ID NO: 33 - protein, 32 aa, Artificial Sequence
  source: /note=“Description of Artificial Sequence Synthetic polypeptide”
KATLSAVTSPNTAYLQMNSLKTEDTAVYYCTT

SEQ ID NO: 34 - DNA, 336 bp, Artificial Sequence
  source: /note=“Description of Artificial Sequence Synthetic polynucleotide”
gacatccagatgacccagtccccctcctccctgtccgcctccgtgggcgacagggtgaccatgacctgcaagtcctcccagtccgtgctgtacagctccaaccagaagaactacctggcctggtaccagcagaagcccggcaaggcccccaagctgctgatctactgggcctccaccagggagtccggcgtgcccgacaggttctccggctccggctccggcaccgacttcaccctgaccatctcctccctgcagcccgaggacttcgccacctactactgccaccagtacctctcgagcctgaccttcggccagggcaccaagctggagatcaag

SEQ ID NO: 35 - protein, 19 aa, Artificial Sequence
  source: /note=“Description of Artificial Sequence Synthetic peptide”
RIKSKTDGGTTDYAAPVKG

SEQ ID NO: 36 - protein, 23 aa, Artificial Sequence
  source: /note=“Description of Artificial Sequence Synthetic peptide”
DIQMTQSPSSVSASVGDRVTITC

SEQ ID NO: 37 - protein, 15 aa, Artificial Sequence
  source: /note=“Description of Artificial Sequence Synthetic peptide”
WYQQKPGKAPKLLIY

SEQ ID NO: 38 - protein, 32 aa, Artificial Sequence
  source: /note=“Description of Artificial Sequence Synthetic polypeptide”
GVPSRFSGSGSGTDFTLTISSLQPEDFATYYC

SEQ ID NO: 39 - protein, 10 aa, Artificial Sequence
  source: /note=“Description of Artificial Sequence Synthetic peptide”
FGQGTKVEIK

SEQ ID NO: 40 - protein, 23 aa, Artificial Sequence
  source: /note=“Description of Artificial Sequence Synthetic peptide”
DIQMTQSPSSLSASVGDRVTMTC

SEQ ID NO: 41 - protein, 32 aa, Artificial Sequence
  source: /note=“Description of Artificial Sequence Synthetic polypeptide”
GVPDRFSGSGSGTDFTLTISSLQPEDFATYYC

SEQ ID NO: 42 - protein, 10 aa, Artificial Sequence
  source: /note=“Description of Artificial Sequence Synthetic peptide”
FGQGTKLEIK

SEQ ID NO: 43 - protein, 23 aa, Artificial Sequence
  source: /note=“Description of Artificial Sequence Synthetic peptide”
NIQMTQSPSSLSASVGDRVTMSC

SEQ ID NO: 44 - protein, 15 aa, Artificial Sequence
  source: /note=“Description of Artificial Sequence Synthetic peptide”
WYQQKPGKSPKLLIY

SEQ ID NO: 45 - protein, 32 aa, Artificial Sequence
  source: /note=“Description of Artificial Sequence Synthetic polypeptide”
GVPDRFTGSGSGTDFTLTISSLQPEDFATYYC

SEQ ID NO: 46 - protein, 23 aa, Artificial Sequence
  source: /note=“Description of Artificial Sequence Synthetic peptide”
DIQMTQSPSSLTVSVGDRVTMSC

SEQ ID NO: 47 - protein, 32 aa, Artificial Sequence
  source: /note=“Description of Artificial Sequence Synthetic polypeptide”
GVPDRFTGSGSGTDFTLTISSVQPEDFATYYC

SEQ ID NO: 48 - protein, 10 aa, Artificial Sequence
  source: /note=“Description of Artificial Sequence Synthetic peptide”
FGAGTKLEIK

SEQ ID NO: 49 - protein, 7 aa, Artificial Sequence
  source: /note=“Description of Artificial Sequence Synthetic peptide”
AASSLQS

SEQ ID NO: 50 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 51 - protein, 799 aa, Homo sapiens
MPRAPAPLYACLLGLCALLPRLAGLNICTSGSATSCEECLLIHPKCAWCSKEDFGSPRSITSRCDLRANLVKNGCGGEIESPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTCGCSVGLEPNSARCNGSGTYVCGLCECSPGYLGTRCECQDGENQSVYQNLCREAEGKPLCSGRGDCSCNQCSCFESEFGKIYGPFCECDNFSCARNKGVLCSGHGECHCGECKCHAGYIGDNCNCSTDISTCRGRDGQICSERGHCLCGQCQCTEPGAFGEMCEKCPTCPDACSTKRDCVECLLLHSGKPDNQTCHSLCRDEVITWVDTIVKDDQEAVLCFYKTAKDCVMMFTYVELPSGKSNLTVLREPECGNTPNAMTILLAVVGSILLVGLALLAIWKLLVTIHDRREFAKFQSERSRARYEMASNPLYRKPISTHTVDFTFNKFNKSYNGTVD

SEQ ID NO: 52 - protein, 799 aa, Mus musculus
MPRVPATLYACLLGLCALVPRLAGLNICTSGSATSCEECLLIHPKCAWCSKEYFGNPRSITSRCDLKANLIRNGCEGEIESPASSTHVLRNLPLSSKGSSATGSDVIQMTPQEIAVSLRPGEQTTFQLQVRQVEDYPVDLYYLMDLSLSMKDDLENIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILHGDSKNIIQLIINAYSSIRAKVELSVWDQPEDLNLFFTATCQDGISYPGQRKCEGLKIGDTASFEVSVEARSCPGRQAAQSFTLRPVGFRDSLQVEVAYNCTCGCSTGLEPNSARCSGNGTYTCGLCECDPGYLGTRCECQEGENQSGYQNLCREAEGKPLCSGRGECSCNQCSCFESEFGRIYGPFCECDSFSCARNKGVLCSGHGECHCGECKCHAGYIGDNCNCSTDVSTCRAKDGQICSDRGRCVCGQCQCTEPGAFGETCEKCPTCPDACSSKRDCVECLLLHQGKPDNQTCHHQCKDEVITWVDTIVKDDQEAVLCFYKTAKDCVMMFSYTELPNGRSNLTVLREPECGSAPNAMTILLAVVGSILLIGMALLAIWKLLVTIHDRREFAKFQSERSRARYEMASNPLYRKPISTHTVDFAFNKFNKSYNGSVD

SEQ ID NO: 53 - DNA, 336 bp, Artificial Sequence
  source: /note=“Description of Artificial Sequence Synthetic polynucleotide”
aacatccagatgacccagtccccctcctccctgtccgcctccgtgggcgacagggtgaccatgtcctgcaagtcctcccagtccgtgctgtacagctccaaccagaagaactacctggcctggtaccagcagaagcccggcaagtcccccaagctgctgatctactgggcctccaccagggagtccggcgtgcccgacaggttcaccggctccggctccggcaccgacttcaccctgaccatctcctccctgcagcccgaggacttcgccacctactactgccaccagtacctctcgagcctgaccttcggccagggcaccaagctggagatcaag

SEQ ID NO: 54 - DNA, 336 bp, Artificial Sequence
  source: /note=“Description of Artificial Sequence Synthetic polynucleotide”
gacatccagatgacccagtccccctcctccctgaccgtgtccgtgggcgacagggtgaccatgtcctgcaagtcctcccagtccgtgctgtacagctccaaccagaagaactacctggcctggtaccagcagaagcccggcaagtcccccaagctgctgatctactgggcctccaccagggagtccggcgtgcccgacaggttcaccggctccggctccggcaccgacttcaccctgaccatctcctccgtgcagcccgaggacttcgccacctactactgccaccagtacctctcgagcctgaccttcggcgccggcaccaagctggagatcaag

SEQ ID NO: 55 - DNA, 336 bp, Artificial Sequence
  source: /note=“Description of Artificial Sequence Synthetic polynucleotide”
gacatccagatgacccagtccccctcctccctgtccgcctccgtgggcgacagggtgaccatgacctgcaagtcctcccagtccgtgctgtacagctccaaccagaagaactacctggcctggtaccagcagaagcccggcaaggcccccaagctgctgatctacgccgcctcctccctgcagtccggcgtgccctccaggttctccggctccggctccggcaccgacttcaccctgaccatctcctccctgcagcccgaggacttcgccacctactactgccaccagtacctctcgagcctgaccttcggccagggcaccaagctggagatcaag

SEQ ID NO: 56 - protein, 107 aa, Artificial Sequence [sequence omitted]

SEQ ID NO: 57 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 7 aa, Artificial Sequence
  source: /note=“Description of Artificial Sequence Synthetic peptide”
GYTFTSY

SEQ ID NO: 59 - protein, 6 aa, Artificial Sequence
  source: /note=“Description of Artificial Sequence Synthetic peptide”
YPGNSD

SEQ ID NO: 60 - protein, 10 aa, Artificial Sequence
  source: /note=“Description of Artificial Sequence Synthetic peptide”
GYTFTSYWMH

SEQ ID NO: 61 - protein, 10 aa, Artificial Sequence
  source: /note=“Description of Artificial Sequence Synthetic peptide”
AIYPGNSDTS

SEQ ID NO: 62 - protein, 6 aa, Artificial Sequence
  source: /note=“Description of Artificial Sequence Synthetic peptide”
TSYWMH

SEQ ID NO: 63 - protein, 13 aa, Artificial Sequence
  source: /note=“Description of Artificial Sequence Synthetic peptide”
WIGAIYPGNSDTS

SEQ ID NO: 64 - protein, 11 aa, Artificial Sequence
  source: /note=“Description of Artificial Sequence Synthetic peptide”
TTTTYGYDWFA

SEQ ID NO: 65 - protein, 13 aa, Artificial Sequence
  source: /note=“Description of Artificial Sequence Synthetic peptide”
LYSSNQKNYLAWY

SEQ ID NO: 66 - protein, 10 aa, Artificial Sequence
  source: /note=“Description of Artificial Sequence Synthetic peptide”
LLIYWASTRE

SEQ ID NO: 67 - protein, 7 aa, Artificial Sequence
  source: /note=“Description of Artificial Sequence Synthetic peptide”
HQYLSSL

SEQ ID NO: 68 - protein, 4 aa, Artificial Sequence
  source: /note=“Description of Artificial Sequence Synthetic peptide”
GGGS

SEQ ID NO: 69 - protein, 445 aa, Artificial Sequence
  source: /note=“Description of Artificial Sequence Synthetic polypeptide”
EVQVVQSGGGLVKPGESLRLSCKASGYTFTSYWMHWVKQAPGKGLEWVGAIYPGNSDTSYNQKFKGKFTISADTSPNTAYLQMNSLKTEDTAVYYCTTTTYGYDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 219 aa, Artificial Sequence
  source: /note=“Description of Artificial Sequence Synthetic polypeptide”
NIQMTQSPSSLSASVGDRVTMSCKSSQSVLYSSNQKNYLAWYQQKPGKSPKLLIYWASTRESGVPDRFTGSGSGTDFTLTISSLQPEDFATYYCHQYLSSLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 119 aa, Mus musculus
EVQVQQSGTVLARPGASVKMSCKASGYTFTSYWMHWVKQRPGQGLEWIGAIYPGNSDTSYNQKFKGKAKLTAVTSPNTAYMELSSLTNEDSAVYYCTTTTYGYDWFAYWGQGTLVTVSA

SEQ ID NO: 72 - protein, 112 aa, Mus musculus
NIMMTQSPSSLTVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELK

SEQ ID NO: 73 - protein, 4 aa, Artificial Sequence
  source: /note=“Description of Artificial Sequence Synthetic peptide”
SGGG

SEQ ID NO: 74 - protein, 5 aa, Artificial Sequence
  source: /note=“Description of Artificial Sequence Synthetic peptide”
GGGGS

SEQ ID NO: 75 - protein, 5 aa, Artificial Sequence
  source: /note=“Description of Artificial Sequence Synthetic peptide”
SGGGG

SEQ ID NO: 76 - protein, 6 aa, Artificial Sequence
  source: /note=“Description of Artificial Sequence Synthetic peptide”
GGGGGS

SEQ ID NO: 77 - protein, 6 aa, Artificial Sequence
  source: /note=“Description of Artificial Sequence Synthetic peptide”
SGGGGG

SEQ ID NO: 78 - protein, 7 aa, Artificial Sequence
  source: /note=“Description of Artificial Sequence Synthetic peptide”
GGGGGGS

SEQ ID NO: 79 - protein, 7 aa, Artificial Sequence
  source: /note=“Description of Artificial Sequence Synthetic peptide”
SGGGGGG

SEQ ID NO: 80 - protein, 445 aa, Artificial Sequence
  source: /note=“Description of Artificial Sequence Synthetic polypeptide”
EVQVVQSGGGLVKPGESLRLSCAASGYTFTSYWMHWVKQAPGKGLEWVGAIYPGNSDTSYNQKFKGKFTISADTSSNTAYLQMNSLKTEDTAVYYCTTTTYGYDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 445 aa, Artificial Sequence
  source: /note=“Description of Artificial Sequence Synthetic polypeptide”
EVQVQQSGGGLVKPGGSLRLSCKASGYTFTSYWMHWVKQAPGKGLEWVGAIYPGNSDTSYNQKFKGKATLSAVTSPNTAYLQMNSLKTEDTAVYYCTTTTYGYDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 219 aa, Artificial Sequence
  source: /note=“Description of Artificial Sequence Synthetic polypeptide”
DIQMTQSPSSVSASVGDRVTITCKSSQSVLYSSNQKNYLAWYQQKPGKAPKLLIYWASTRESGVPSRFSGSGSGTDFTLTISSLQPEDFATYYCHQYLSSLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - DNA, 1338 bp, Artificial Sequence
  source: /note=“Description of Artificial Sequence Synthetic polynucleotide”
gaggtgcagcttgtggagtccggcggcggcctggtgaagcccggcggctccctgaggctgtcctgcgccgcctccggctacaccttcacctcctactggatgcactgggtgaggcaggcccccggcaagggcctggagtgggtgggcgccatctaccccgggaattctgacacctcctacaaccagaagttcaagggcaggttcaccatctccagggacgactccaagaacaccctgtacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 84 - DNA, 1338 bp, Artificial Sequence
  source: /note=“Description of Artificial Sequence Synthetic polynucleotide”
gaggtgcaggtggtggagtccggcggcggcctggtgaagcccggcggctccctgaggctgtcctgcgccgcctccggctacaccttcacctcctactggatgcactgggtgaggcaggcccccggcaagggcctggagtgggtgggcgccatctaccccgggaattctgacacctcctacaaccagaagttcaagggcaggttcaccatctccgccgacacctccaagaacaccctgtacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 85 - DNA, 1338 bp, Artificial Sequence
  source: /note=“Description of Artificial Sequence Synthetic polynucleotide”
gaggtgcaggtggtgcagtccggcggcggcctggtgaagcccggcgagtccctgaggctgtcctgcgccgcctccggctacaccttcacctcctactggatgcactgggtgaagcaggcccccggcaagggcctggagtgggtgggcgccatctaccccgggaattctgacacctcctacaaccagaagttcaagggcaagttcaccatctccgccgacacctcctccaacaccgcctacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 86 - DNA, 1338 bp, Artificial Sequence
  source: /note=“Description of Artificial Sequence Synthetic polynucleotide”
gaggtgcaggtggtggagtccggcggcggcctggtgaagcccggcggctccctgaggctgtcctgcaaggcctccggctacaccttcacctcctactggatgcactgggtgaagcaggcccccggcaagggcctggagtgggtgggcgccatctaccccgggaattctgacacctcctacaaccagaagttcaagggcaagttcaccctgtccgccgtgacctcctccaacaccgcctacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 87 - DNA, 1338 bp, Artificial Sequence
  source: /note=“Description of Artificial Sequence Synthetic polynucleotide”
gaggtgcaggtggtgcagtccggcggcggcctggtgaagcccggcgagtccctgaggctgtcctgcaaggcctccggctacaccttcacctcctactggatgcactgggtgaagcaggcccccggcaagggcctggagtgggtgggcgccatctaccccgggaattctgacacctcctacaaccagaagttcaagggcaagttcaccatctccgccgacacctcccccaacaccgcctacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 88 - DNA, 1338 bp, Artificial Sequence
  source: /note=“Description of Artificial Sequence Synthetic polynucleotide”
gaggtgcaggtgcagcagtccggcggcggcctggtgaagcccggcggctccctgaggctgtcctgcaaggcctccggctacaccttcacctcctactggatgcactgggtgaagcaggcccccggcaagggcctggagtgggtgggcgccatctaccccgggaattctgacacctcctacaaccagaagttcaagggcaaggccaccctgtccgccgtgacctcccccaacaccgcctacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 89 - DNA, 1344 bp, Artificial Sequence
  source: /note=“Description of Artificial Sequence Synthetic polynucleotide”
gaggtgcaggtggtggagtccggcggcggcctggtgaagcccggcggctccctgaggctgtcctgcgccgcctccggctacaccttcacctcctactggatgcactgggtgaggcaggcccccggcaagggcctggagtgggtgggcaggatcaagtccaagaccgacggcggtaccaccgactacgccgcccccgtgaagggcaggttcaccatctccgccgacacctccaagaacaccctgtacctgcagatgaactccctgaagaccgaggacaccgccgtgtactactgcaccaccaccacctacggctacgactggttcgcctactggggccagggcaccctggtgaccgtctcgagcgcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaagagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 90 - DNA, 660 bp, Artificial Sequence
  source: /note=“Description of Artificial Sequence Synthetic polynucleotide”
gacatccagatgacccagtccccctcctccgtgtccgcctccgtgggcgacagggtgaccatcacctgcaagtcctcccagtccgtgctgtacagctccaaccagaagaactacctggcctggtaccagcagaagcccggcaaggcccccaagctgctgatctactgggcctccaccagggagtccggcgtgccctccaggttctccggctccggctccggcaccgacttcaccctgaccatctcctccctgcagcccgaggacttcgccacctactactgccaccagtacctctcgagcctgaccttcggccagggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 91 - DNA, 660 bp, Artificial Sequence
  source: /note=“Description of Artificial Sequence Synthetic polynucleotide”
gacatccagatgacccagtccccctcctccctgtccgcctccgtgggcgacagggtgaccatgacctgcaagtcctcccagtccgtgctgtacagctccaaccagaagaactacctggcctggtaccagcagaagcccggcaaggcccccaagctgctgatctactgggcctccaccagggagtccggcgtgcccgacaggttctccggctccggctccggcaccgacttcaccctgaccatctcctccctgcagcccgaggacttcgccacctactactgccaccagtacctctcgagcctgaccttcggccagggcaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 92 - DNA, 660 bp, Artificial Sequence
  source: /note=“Description of Artificial Sequence Synthetic polynucleotide”
aacatccagatgacccagtccccctcctccctgtccgcctccgtgggcgacagggtgaccatgtcctgcaagtcctcccagtccgtgctgtacagctccaaccagaagaactacctggcctggtaccagcagaagcccggcaagtcccccaagctgctgatctactgggcctccaccagggagtccggcgtgcccgacaggttcaccggctccggctccggcaccgacttcaccctgaccatctcctccctgcagcccgaggacttcgccacctactactgccaccagtacctctcgagcctgaccttcggccagggcaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 93 - DNA, 660 bp, Artificial Sequence
  source: /note=“Description of Artificial Sequence Synthetic polynucleotide”
gacatccagatgacccagtccccctcctccctgaccgtgtccgtgggcgacagggtgaccatgtcctgcaagtcctcccagtccgtgctgtacagctccaaccagaagaactacctggcctggtaccagcagaagcccggcaagtcccccaagctgctgatctactgggcctccaccagggagtccggcgtgcccgacaggttcaccggctccggctccggcaccgacttcaccctgaccatctcctccgtgcagcccgaggacttcgccacctactactgccaccagtacctctcgagcctgaccttcggcgccggcaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 94 - DNA, 660 bp, Artificial Sequence
  source: /note=“Description of Artificial Sequence Synthetic polynucleotide”
gacatccagatgacccagtccccctcctccctgtccgcctccgtgggcgacagggtgaccatgacctgcaagtcctcccagtccgtgctgtacagctccaaccagaagaactacctggcctggtaccagcagaagcccggcaaggcccccaagctgctgatctacgccgcctcctccctgcagtccggcgtgccctccaggttctccggctccggctccggcaccgacttcaccctgaccatctcctccctgcagcccgaggacttcgccacctactactgccaccagtacctctcgagcctgaccttcggccagggcaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 95 - DNA, 357 bp, Artificial Sequence
  source: /note=“Description of Artificial Sequence Synthetic polynucleotide”
gaggtgcagcttgtggagtccggcggcggcctggtgaagcccggcggctccctgaggctgtcctgcgccgcctccggctacaccttcacctcctactggatgcactgggtgaggcaggcccccggcaagggcctggagtgggtgggcgccatctaccccgggaattctgacacctcctacaaccagaagttcaagggcaggttcaccatctccagggacgactccaagaacaccctgtacctgcagatgaactccctgaagaccgaggacaccgccgtgtactactgcaccaccaccacctacggctacgactggttcgcctactggggccagggcaccctggtgaccgtctcgagc

SEQ ID NO: 96 - DNA, 357 bp, Artificial Sequence
  source: /note=“Description of Artificial Sequence Synthetic polynucleotide”
gaggtgcaggtggtggagtccggcggcggcctggtgaagcccggcggctccctgaggctgtcctgcgccgcctccggctacaccttcacctcctactggatgcactgggtgaggcaggcccccggcaagggcctggagtgggtgggcgccatctaccccgggaattctgacacctcctacaaccagaagttcaagggcaggttcaccatctccgccgacacctccaagaacaccctgtacctgcagatgaactccctgaagaccgaggacaccgccgtgtactactgcaccaccaccacctacggctacgactggttcgcctactggggccagggcaccctggtgaccgtctcgagc

SEQ ID NO: 97 - DNA, 357 bp, Artificial Sequence
  source: /note=“Description of Artificial Sequence Synthetic polynucleotide”
gaggtgcaggtggtgcagtccggcggcggcctggtgaagcccggcgagtccctgaggctgtcctgcgccgcctccggctacaccttcacctcctactggatgcactgggtgaagcaggcccccggcaagggcctggagtgggtgggcgccatctaccccgggaattctgacacctcctacaaccagaagttcaagggcaagttcaccatctccgccgacacctcctccaacaccgcctacctgcagatgaactccctgaagaccgaggacaccgccgtgtactactgcaccaccaccacctacggctacgactggttcgcctactggggccagggcaccctggtgaccgtctcgagc

SEQ ID NO: 98 - DNA, 357 bp, Artificial Sequence
  source: /note=“Description of Artificial Sequence Synthetic polynucleotide”
gaggtgcaggtggtggagtccggcggcggcctggtgaagcccggcggctccctgaggctgtcctgcaaggcctccggctacaccttcacctcctactggatgcactgggtgaagcaggcccccggcaagggcctggagtgggtgggcgccatctaccccgggaattctgacacctcctacaaccagaagttcaagggcaagttcaccctgtccgccgtgacctcctccaacaccgcctacctgcagatgaactccctgaagaccgaggacaccgccgtgtactactgcaccaccaccacctacggctacgactggttcgcctactggggccagggcaccctggtgaccgtctcgagc

SEQ ID NO: 99 - DNA, 357 bp, Artificial Sequence
  source: /note=“Description of Artificial Sequence Synthetic polynucleotide”
gaggtgcaggtggtgcagtccggcggcggcctggtgaagcccggcgagtccctgaggctgtcctgcaaggcctccggctacaccttcacctcctactggatgcactgggtgaagcaggcccccggcaagggcctggagtgggtgggcgccatctaccccgggaattctgacacctcctacaaccagaagttcaagggcaagttcaccatctccgccgacacctcccccaacaccgcctacctgcagatgaactccctgaagaccgaggacaccgccgtgtactactgcaccaccaccacctacggctacgactggttcgcctactggggccagggcaccctggtgaccgtctcgagc

SEQ ID NO: 100 - DNA, 357 bp, Artificial Sequence
  source: /note=“Description of Artificial Sequence Synthetic polynucleotide”
gaggtgcaggtgcagcagtccggcggcggcctggtgaagcccggcggctccctgaggctgtcctgcaaggcctccggctacaccttcacctcctactggatgcactgggtgaagcaggcccccggcaagggcctggagtgggtgggcgccatctaccccgggaattctgacacctcctacaaccagaagttcaagggcaaggccaccctgtccgccgtgacctcccccaacaccgcctacctgcagatgaactccctgaagaccgaggacaccgccgtgtactactgcaccaccaccacctacggctacgactggttcgcctactggggccagggcaccctggtgaccgtctcgagc

SEQ ID NO: 101 - DNA, 363 bp, Artificial Sequence
  source: /note=“Description of Artificial Sequence Synthetic polynucleotide”
gaggtgcaggtggtggagtccggcggcggcctggtgaagcccggcggctccctgaggctgtcctgcgccgcctccggctacaccttcacctcctactggatgcactgggtgaggcaggcccccggcaagggcctggagtgggtgggcaggatcaagtccaagaccgacggcggtaccaccgactacgccgcccccgtgaagggcaggttcaccatctccgccgacacctccaagaacaccctgtacctgcagatgaactccctgaagaccgaggacaccgccgtgtactactgcaccaccaccacctacggctacgactggttcgcctactggggccagggcaccctggtgaccgtctcgagc

SEQ ID NO: 102 - DNA, 336 bp, Artificial Sequence
  source: /note=“Description of Artificial Sequence Synthetic polynucleotide”
gacatccagatgacccagtccccctcctccgtgtccgcctccgtgggcgacagggtgaccatcacctgcaagtcctcccagtccgtgctgtacagctccaaccagaagaactacctggcctggtaccagcagaagcccggcaaggcccccaagctgctgatctactgggcctccaccagggagtccggcgtgccctccaggttctccggctccggctccggcaccgacttcaccctgaccatctcctccctgcagcccgaggacttcgccacctactactgccaccagtacctctcgagcctgaccttcggccagggcaccaaggtggagatcaag